(19) United States
(12) Patent Application Publication
(10) Pub. No.: US 2026/0165027 A1
(43) Pub. Date: Jun. 11, 2026

(54) COMPOUND HAVING UNSHARED ELECTRON PAIR FOR PEROVSKITE SOLAR CELL, PEROVSKITE SOLAR CELL HAVING THE SAME AND MANUFACTURING METHOD THEREOF

(71) Applicant: HANWHA SOLUTIONS CORPORATION, Seoul (KR)

(72) Inventors: Dongki JUNG, Seoul (KR); Eunsoo JANG, Seoul (KR); In Taek CHOI, Seoul (KR)

(73) Assignee: HANWHA SOLUTIONS CORPORATION, Seoul (KR)

(21) Appl. No.: 19/382,990

(22) Filed: Nov. 7, 2025

(30) Foreign Application Priority Data
Dec. 11, 2024 (KR) 10-2024-0183496
May 28, 2025 (KR) 10-2025-0069782
May 28, 2025 (KR) 10-2025-0069783
May 28, 2025 (KR) 10-2025-0069784
May 28, 2025 (KR) 10-2025-0069785

(51) Int. Cl. H10K 85/60; C07D 471/22; C07F 5/02; C07F 9/6561; H10K 30/40; H10K 30/57

(52) U.S. Cl. CPC H10K 85/6572; C07D 471/22; C07F 5/025; C07F 9/6561; H10K 30/40; H10K 30/57

(57) ABSTRACT

The present disclosure relates to a novel compound having an unshared electron pair for a perovskite solar cell, a perovskite solar cell including the same, and a method of manufacturing the same.

### CROSS-REFERENCE TO RELATED APPLICATIONS

The present application claims priority under 35 U.S.C. § 119 (a) to Korean patent application number 10-2024-0183496 filed on Dec. 11, 2024, 10-2025-0069782, 10-2025-0069783, 10-2025-0069784, and 10-2025-0069785 filed on May 28, 2025, in the Korean Intellectual Property Office, the entire disclosures of which are incorporated by reference herein.

### BACKGROUND

### 1. Technical Field

The present disclosure relates to a novel compound having an unshared electron pair for a perovskite solar cell, a perovskite solar cell including the same, and a method of manufacturing the same.

### 2. Related Art

In order to solve global environmental problems caused by the depletion and use of fossil energy, research on renewable and clean alternative energy sources such as solar energy, wind power, and hydroelectric power is being actively conducted. Among them, interest in solar cells that directly convert sunlight into electrical energy is greatly increasing. Here, a solar cell refers to a cell that generates current and voltage by utilizing the photovoltaic effect in which electrons and holes are generated by absorbing light energy from sunlight.

Currently, n-p diode-type silicon (Si) single crystal-based solar cells with a light energy conversion efficiency exceeding 20% can be manufactured and are actually used in solar power generation, and there are also solar cells using compound semiconductors such as gallium arsenide (GaAs), which exhibit even higher conversion efficiency. However, since these inorganic semiconductor-based solar cells require highly purified materials for high efficiency, a large amount of energy is consumed in refining raw materials, and expensive process equipment is also required in the process of forming single crystals or thin films from the raw materials. Therefore, there is a limitation in lowering the manufacturing cost of solar cells, which has been an obstacle to large-scale utilization.

Accordingly, in order to manufacture solar cells at low cost, it is necessary to significantly reduce the cost of materials or manufacturing processes used as key components of solar cells. As an alternative to inorganic semiconductor-based solar cells, research has been conducted on perovskite solar cells, which can be manufactured with low-cost materials and processes.

The general structural formula of the perovskite structure is an ABX₃ structure, in which an anion is located at the X position, a large cation is located at the A position, and a small cation is located at the B position.

Meanwhile, in order for a perovskite solar cell to exhibit excellent efficiency, a material that induces hydrogen bonding between molecules to improve intermolecular interaction and possesses high hole-transport capability is required.

However, the materials conventionally used, even when placed at appropriate positions or in appropriate thin film layers in a perovskite solar cell, had the problem of not being able to achieve the highest efficiency.

### RELATED ART DOCUMENT

### Patent Document

Korean Laid-Open Patent Publication No. 10-2015-0032889 (published 2015 Mar. 30)

### SUMMARY

The present disclosure has been devised to overcome the above-described problems, and is directed to providing a compound having an unshared electron pair for a perovskite solar cell, a perovskite solar cell including the same, and a method for manufacturing the same, which can improve intermolecular interactions by inducing hydrogen bonding between molecules and secure an energy level suitable for hole transport.

In order to solve the above-described problems, the present disclosure provides a compound having an unshared electron pair for a perovskite solar cell represented by [Chemical Formula 1] below.

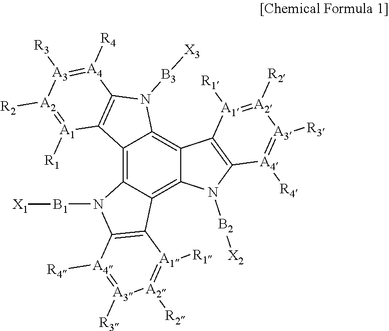

In [Chemical Formula 1], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a boronic acid group (—B(OH)₂), a boronic ester group, a dihydroxyphosphoryl group (—P═O(OH)₂), a trihalosilyl group (—SiX₃), a trialkoxysilyl group (—Si(OR)₃), a carboxyl group (—COOH), or a sulfo group (—SO₃H).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-25] below.

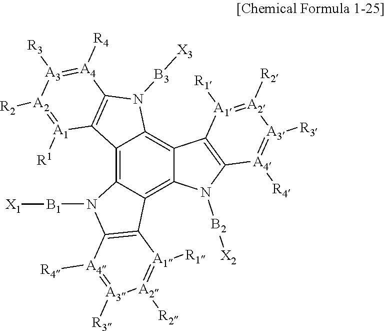

In [Chemical Formula 1-25], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, —CH₂CH₂CH₂CH₂—, —CH₂CH₂CH₂CH₂CH₂—,

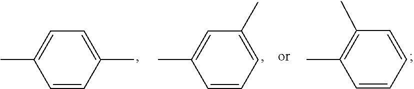

X₁, X₂, and X₃ are each independently a boronic acid group (—B(OH)₂),

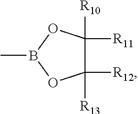

a dihydroxyphosphoryl group (—P═O(OH)₂), a carboxyl group (—COOH), or a sulfo group (—SO₃H); and R₁₀, R₁₁, R₁₂, and R₁₃ are each independently —H, a C1-C20 linear alkyl group, or a C3-C20 branched alkyl group.

In addition, the compound having an unshared electron pair for a perovskite solar cell of the present disclosure may include a reaction product of a third reaction product and hydrochloric acid (HCl). The third reaction product may be obtained as a reaction product of a second reaction product and trimethyl borate, and the second reaction product may be obtained as a reaction product of a compound represented by [Chemical Formula 2], sodium hydride, and dibromobenzene.

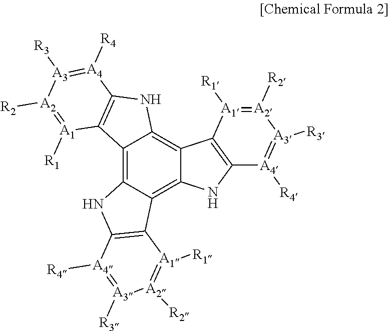

In [Chemical Formula 2], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N), and R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group.

In addition, the compound having an unshared electron pair for a perovskite solar cell of the present disclosure may include a reaction product of a second reaction product and bromotrimethylsilane, and the second reaction product may be a reaction product of the compound represented by [Chemical Formula 2], sodium hydride, and diethyl(3-bromopropyl)phosphonate.

In addition, the compound having an unshared electron pair for a perovskite solar cell of the present disclosure may include a reaction product of a second reaction product and sodium hydroxide (NaOH), and the second reaction product may be a reaction product of the compound represented by [Chemical Formula 2], ethyl 3-bromopropionate, potassium carbonate, and potassium iodide.

In addition, the compound having an unshared electron pair for a perovskite solar cell of the present disclosure may include a reaction product of a third reaction product and hydrochloric acid (HCl), and the third reaction product may be a reaction product of a second reaction product and sodium sulfite, while the second reaction product may be a reaction product of the compound represented by [Chemical Formula 2], 1,3-dibromoalkane, potassium carbonate, and potassium iodide.

In a preferred embodiment of the present disclosure, the compound represented by [Chemical Formula 2] may include a reaction product of a compound represented by [Chemical Formula 3] below and phosphoryl chloride.

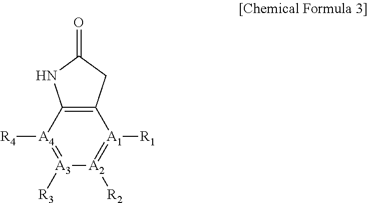

In [Chemical Formula 3], A₁, A₂, A₃, and A₄ are each independently carbon (C) or nitrogen (N), provided that at least one of A₁, A₂, A₃, and A₄ is nitrogen (N), and R₁, R₂, R₃, and R₄ are each independently —H, a C1-C20 linear alkyl group, or a C3-C20 branched alkyl group.

In addition, the compound having an unshared electron pair for a perovskite solar cell of the present disclosure may include a reaction product of a second reaction product, bis(pinacolato)diboron, Pd(dppf)Cl₂, and potassium acetate, and the second reaction product may be a reaction product of a compound represented by [Chemical Formula 2] below, sodium hydride, and dibromobenzene.

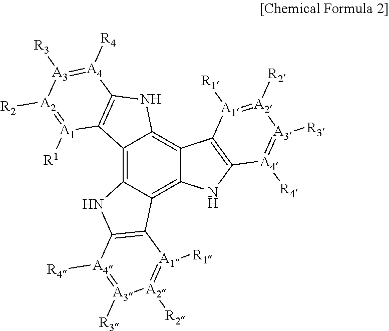

In [Chemical Formula 2], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N), and R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group.

In addition, the compound having an unshared electron pair for a perovskite solar cell of the present disclosure may include a reaction product of a third reaction product and hydrochloric acid (HCl), the third reaction product may be a reaction product of a second reaction product, diethyl phosphite, triphenylphosphine, and cesium carbonate, and the second reaction product may be a reaction product of the compound represented by [Chemical Formula 2], sodium hydride, and 1,4-dibromobenzene.

In a preferred embodiment of the present disclosure, the compound represented by [Chemical Formula 2] may include a reaction product of a compound represented by [Chemical Formula 3] below and phosphoryl chloride.

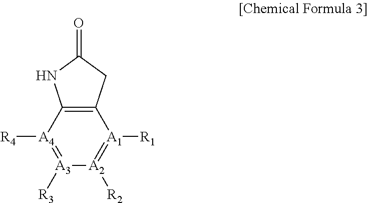

In [Chemical Formula 3], A₁, A₂, A₃, and A₄ are each independently carbon (C) or nitrogen (N), provided that at least one of A₁, A₂, A₃, and A₄ is nitrogen (N), and R₁, R₂, R₃, and R₄ are each independently —H, a C1-C20 linear alkyl group, or a C3-C20 branched alkyl group.

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-1] below.

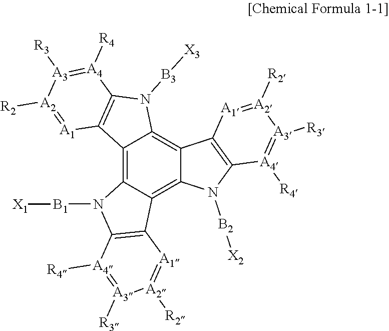

In [Chemical Formula 1-1], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently

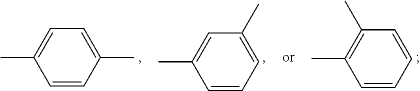

and X₁, X₂, and X₃ are each a boronic acid group (—B(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-2] below.

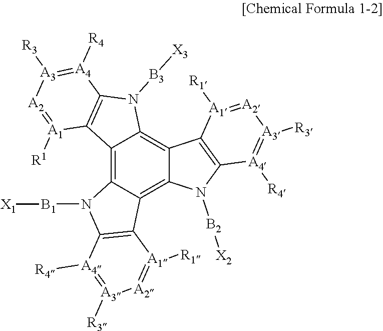

In [Chemical Formula 1-2], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently

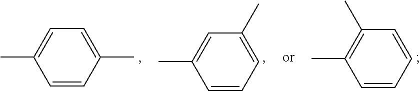

and X₁, X₂, and X₃ are each a boronic acid group (—B(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-3] below.

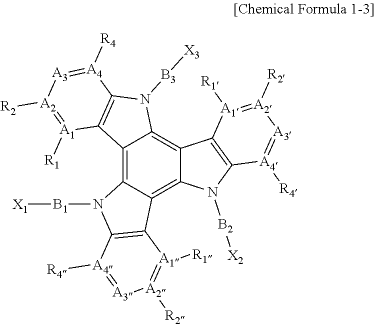

In [Chemical Formula 1-3], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently

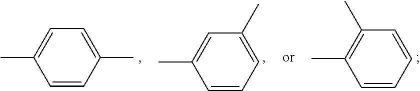

and X₁, X₂, and X₃ are each a boronic acid group (—B(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-4] below.

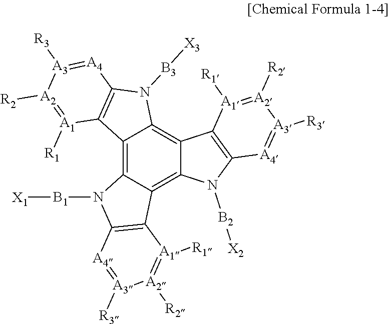

In [Chemical Formula 1-4], A₁, A₂, A₃, A₁, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently

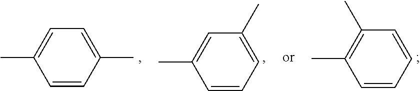

and X₁, X₂, and X₃ are each a boronic acid group (—B(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-6] below.

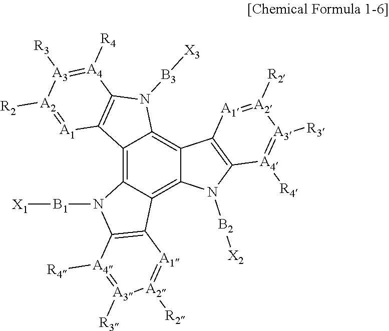

In [Chemical Formula 1-6], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently

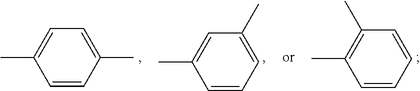

X₁, X₂, and X₃ are each

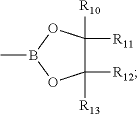

and R₁₀, R₁₁, R₁₂, and R¹³ are each independently —H, a C1-C20 linear alkyl group, or a C3-C20 branched alkyl group.

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-7] below.

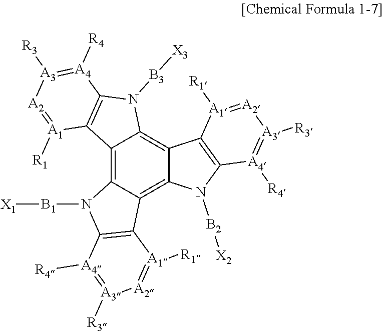

In [Chemical Formula 1-7], A₁, A₃, A₄, A₁, A₃, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently

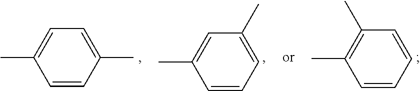

X₁, X₂, and X₃ are each

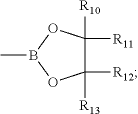

and R₁₀, R₁₁, R₁₂, and R¹³ are each independently —H, a C1-C20 linear alkyl group, or a C3-C20 branched alkyl group.

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-8] below.

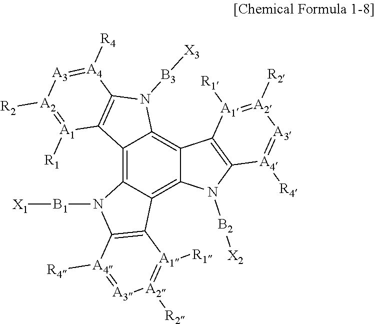

In [Chemical Formula 1-8], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently

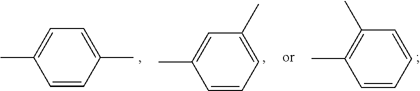

X₁, X₂, and X₃ are each

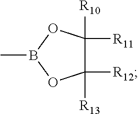

and R₁₀, R₁₁, R₁₂, and R¹³ are each independently —H, a C1-C20 linear alkyl group, or a C3-C20 branched alkyl group.

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-9] below.

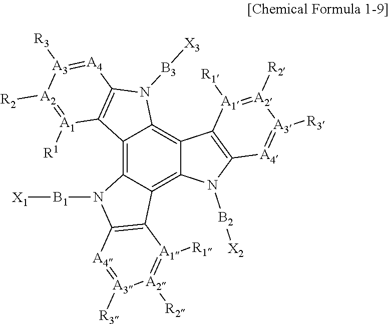

In [Chemical Formula 1-9], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently

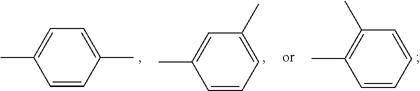

X₁, X₂, and X₃ are each

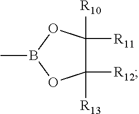

and R₁₀, R₁₁, R₁₂, and R₁₃ are each independently —H, a C1-C20 linear alkyl group, or a C3-C20 branched alkyl group.

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-10] below.

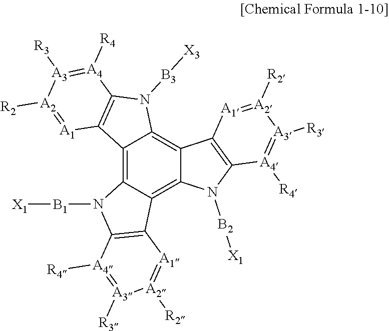

In [Chemical Formula 1-10], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently —CH₂CH₂—, —CH₂CH₂CH₂—, or —CH₂CH₂CH₂CH₂—; and X₁, X₂, and X₃ are each a dihydroxyphosphoryl group (—P═O(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-11] below.

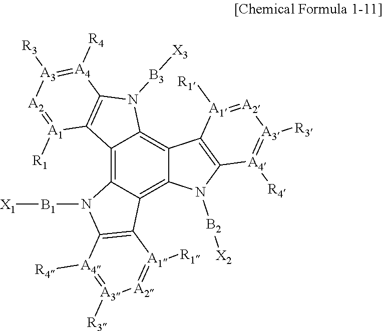

In [Chemical Formula 1-11], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently —CH₂CH₂—, —CH₂CH₂CH₂—, or —CH₂CH₂CH₂CH₂—; and X₁, X₂, and X₃ are each dihydroxyphosphoryl group (—P═O(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-12] below.

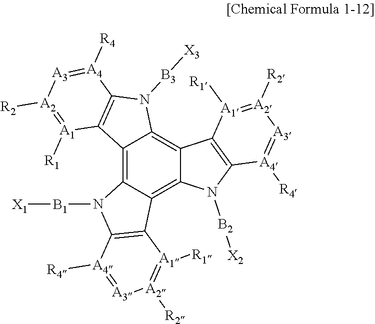

In [Chemical Formula 1-12], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently —CH₂CH₂—, —CH₂CH₂CH₂—, or —CH₂CH₂CH₂CH₂—; and X₁, X₂, and X₃ are each dihydroxyphosphoryl group (—P═O(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-13] below.

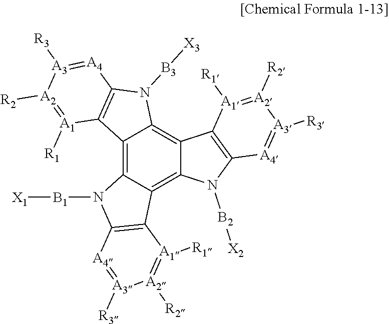

In [Chemical Formula 1-13], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R^{3″} are each independently —H, a C1-C20 linear alkyl group, or a C3-C20 branched alkyl group; B₁, B₂, and B₃ are each independently —CH₂CH₂—, —CH₂CH₂CH₂—, or —CH₂CH₂CH₂CH₂—; and X₁, X₂, and X₃ are each dihydroxyphosphoryl group (—P═O(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-14] below.

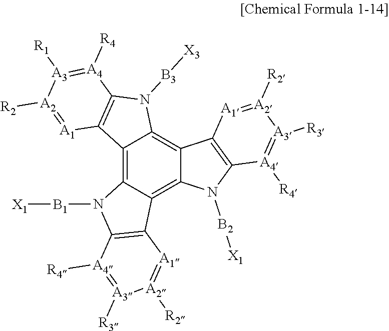

In [Chemical Formula 1-14], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently

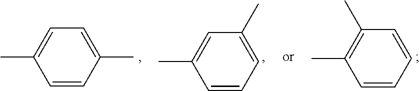

and X₁, X₂, and X₃ are each a dihydroxyphosphoryl group (—P═O(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-15] below.

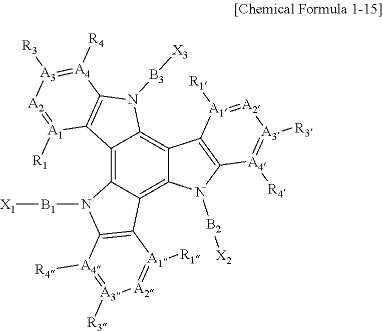

In [Chemical Formula 1-15], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently

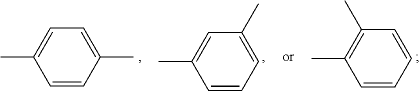

and X₁, X₂, and X₃ are each a dihydroxyphosphoryl group (—P═O(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-16] below.

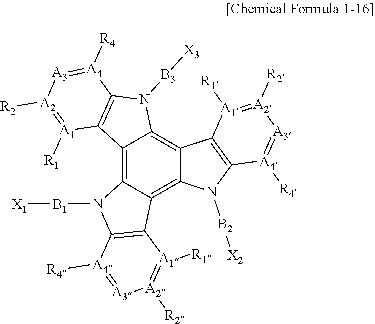

In [Chemical Formula 1-16], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently

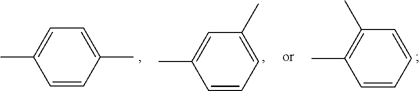

and X₁, X₂, and X₃ are each a dihydroxyphosphoryl group (—P═O(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-17] below.

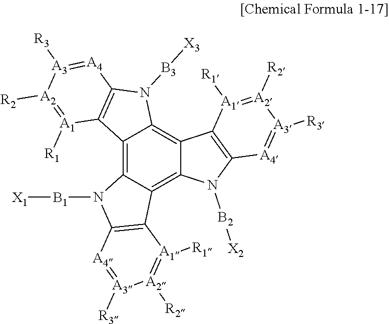

In [Chemical Formula 1-17], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently

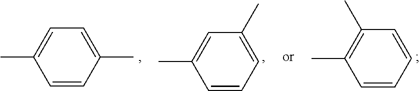

and X₁, X₂, and X₃ are each a dihydroxyphosphoryl group (—P═O(OH)₂).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-18] below.

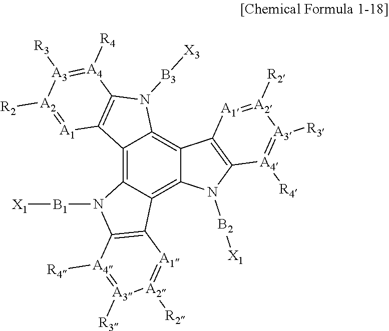

In [Chemical Formula 1-18], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each carboxyl group (—COOH).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-19] below.

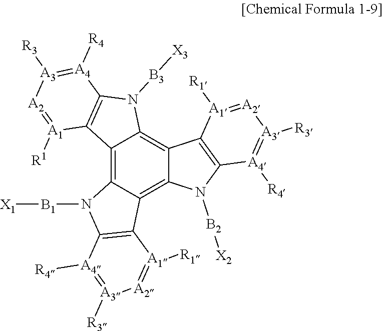

In [Chemical Formula 1-19], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each carboxyl group (—COOH).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-20] below.

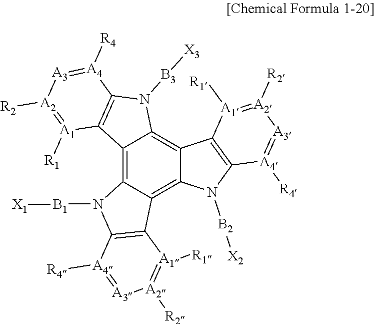

In [Chemical Formula 1-20], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each carboxyl group (—COOH).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-26] below.

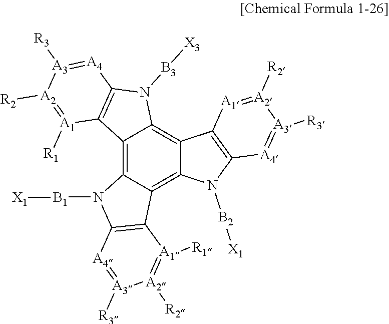

In [Chemical Formula 1-26], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each carboxyl group (—COOH).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-21] below.

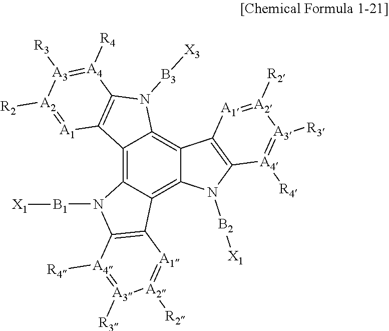

In [Chemical Formula 1-21], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each sulfo group (—SO₃H).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-22] below.

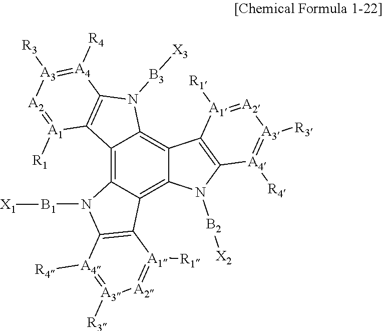

In [Chemical Formula 1-22], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each sulfo group (—SO₃H).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-23] below.

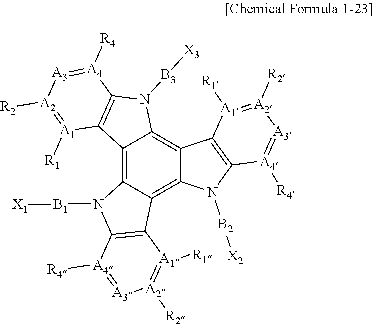

In [Chemical Formula 1-23], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each sulfo group (—SO₃H).

In a preferred embodiment of the present disclosure, the compound having an unshared electron pair for a perovskite solar cell represented by [Chemical Formula 1] may be a compound having an unshared electron pair for a perovskite solar cell represented by [Chemical Formula 1-27] below.

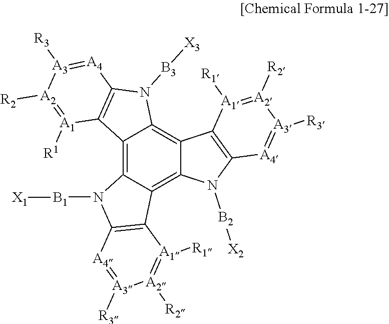

In [Chemical Formula 1-27], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each sulfo group (—SO₃H).

Meanwhile, a method for manufacturing a compound having an unshared electron pair for a perovskite solar cell of the present disclosure may include Step 1 of adding a compound represented by [Chemical Formula 3] below to phosphoryl chloride and reacting them to prepare a compound represented by [Chemical Formula 2] below; Step 2 of adding sodium hydride and dibromobenzene to the compound represented by [Chemical Formula 2] below and reacting them to prepare a second reaction product; Step 3 of adding trimethyl borate to the second reaction product and reacting them to prepare a third reaction product; and Step 4 of adding hydrochloric acid (HCl) to the third reaction product to manufacture a compound having an unshared electron pair for a perovskite solar cell represented by [Chemical Formula 1] below.

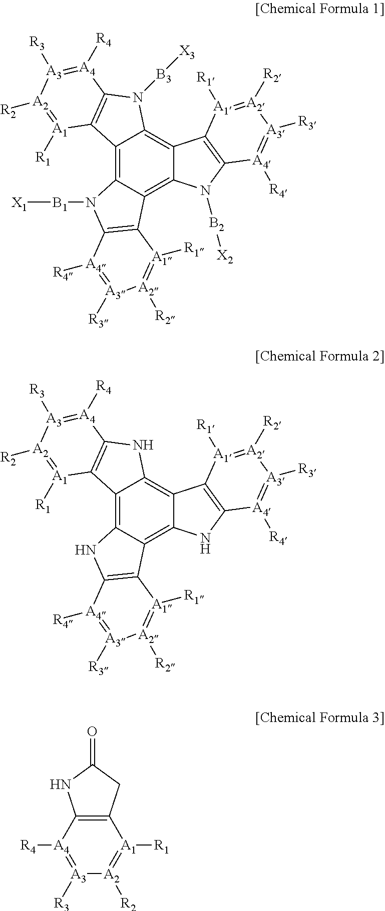

In [Chemical Formulas 1 to 3], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a boronic acid group (—B(OH)₂) or a boronic ester group.

Furthermore, a method of manufacturing a compound having an unshared electron pair for a perovskite solar cell according to the present disclosure may include Step 1 of adding a compound represented by [Chemical Formula 3] below to phosphoryl chloride and reacting them to prepare a compound represented by [Chemical Formula 2] below; Step 2 of adding the compound represented by [Chemical Formula 2] below to sodium hydride and dibromobenzene and reacting them to prepare a second reaction product; and Step 3 of adding the second reaction product to bis(pinacolato)diboron, Pd(dppf)Cl₂, and potassium acetate and reacting them to manufacture a compound having an unshared electron pair for a perovskite solar cell represented by [Chemical Formula 1] below.

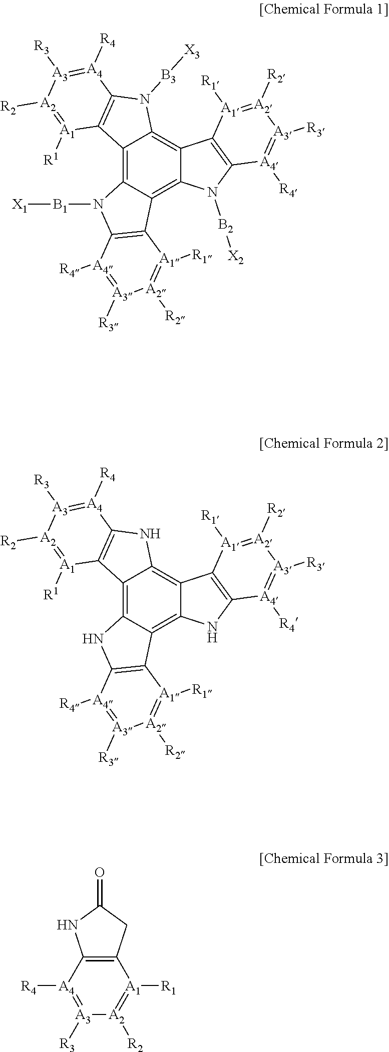

In [Chemical Formulas 1 to 3], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a boronic acid group (—B(OH)₂) or a boronic ester group.

Meanwhile, another method for manufacturing a compound having an unshared electron pair for a perovskite solar cell of the present disclosure may include Step 1 of adding a compound represented by [Chemical Formula 3] below to phosphoryl chloride and reacting them to prepare a compound represented by [Chemical Formula 2] below; Step 2 of adding sodium hydride and diethyl(3-bromopropyl)phosphonate to the compound represented by [Chemical Formula 2] below and reacting them to prepare a second reaction product; and Step 3 of adding bromotrimethylsilane to the second reaction product and reacting them to manufacture a compound having an unshared electron pair for a perovskite solar cell represented by [Chemical Formula 1] below.

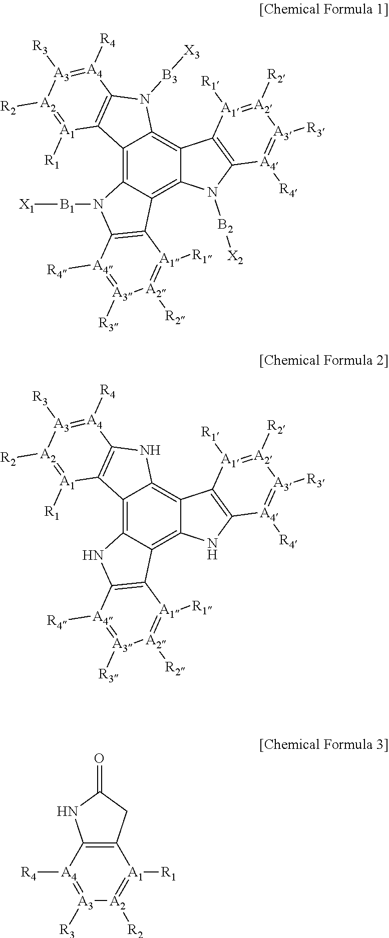

In [Chemical Formulas 1 to 3], A₁, A₂, A₃, A₄, A₁, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a dihydroxyphosphoryl group (—P═O(OH)₂), a trihalosilyl group (—SiX₃), or a trialkoxysilyl group (—Si(OR)₃).

Furthermore, another method for manufacturing a compound having an unshared electron pair for a perovskite solar cell of the present disclosure may include Step 1 of adding a compound represented by [Chemical Formula 3] below to phosphoryl chloride and reacting them to prepare a compound represented by [Chemical Formula 2] below; Step 2 of adding sodium hydride and 1,4-dibromobenzene to the compound represented by [Chemical Formula 2] below and reacting them to prepare a second reaction product; Step 3 of adding diethyl phosphite, triphenylphosphine, and cesium carbonate to the second reaction product and reacting them to prepare a third reaction product; and Step 4 of adding hydrochloric acid (HCl) to the third reaction product to manufacture a compound having an unshared electron pair for a perovskite solar cell represented by [Chemical Formula 1] below.

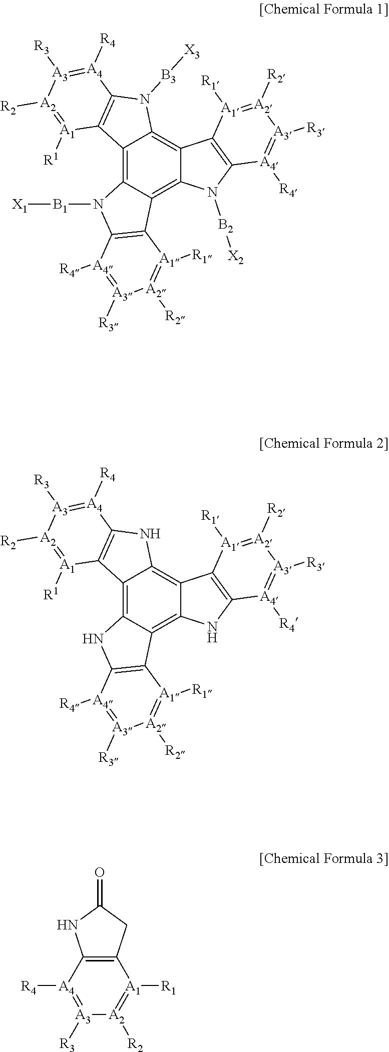

In [Chemical Formulas 1 to 3], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a dihydroxyphosphoryl group (—P═O(OH)₂), a trihalosilyl group (—SiX₃), or a trialkoxysilyl group (—Si(OR)₃).

Meanwhile, still another method for manufacturing a compound having an unshared electron pair for a perovskite solar cell of the present disclosure may include Step 1 of adding a compound represented by [Chemical Formula 3] below to phosphoryl chloride and reacting them to prepare a compound represented by [Chemical Formula 2] below; Step 2 of adding ethyl 3-bromopropionate, potassium carbonate, and potassium iodide to the compound represented by [Chemical Formula 2] below and reacting them to prepare a second reaction product; and Step 3 of adding sodium hydroxide (NaOH) to the second reaction product and reacting them to manufacture a compound having an unshared electron pair for a perovskite solar cell represented by [Chemical Formula 1] below.

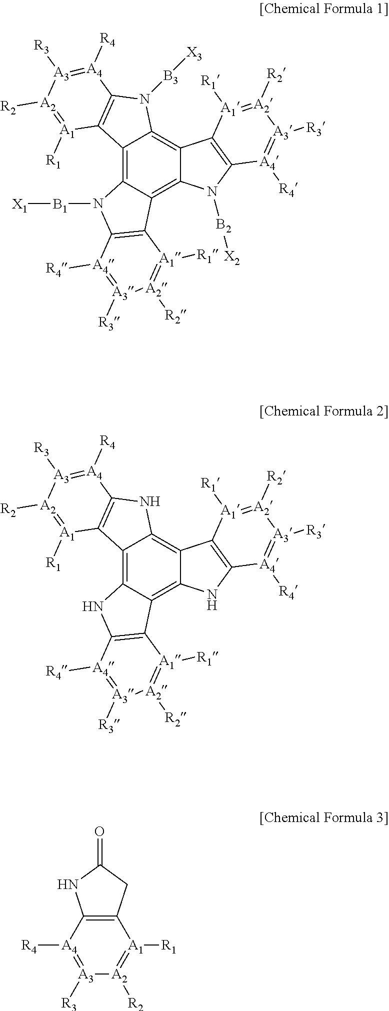

In [Chemical Formulas 1 to 3], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a carboxyl group (—COOH).

Meanwhile, still another method for manufacturing a compound having an unshared electron pair for a perovskite solar cell of the present disclosure may include Step 1 of adding a compound represented by [Chemical Formula 3] below to phosphoryl chloride and reacting them to prepare a compound represented by [Chemical Formula 2] below; Step 2 of adding 1,3-dibromoalkane, potassium carbonate, and potassium iodide to the compound represented by [Chemical Formula 2] below and reacting them to prepare a second reaction product; Step 3 of adding sodium sulfite to the second reaction product and reacting them to prepare a third reaction product; and Step 4 of adding hydrochloric acid (HCl) to the third reaction product to manufacture a compound having an unshared electron pair for a perovskite solar cell represented by [Chemical Formula 1] below.

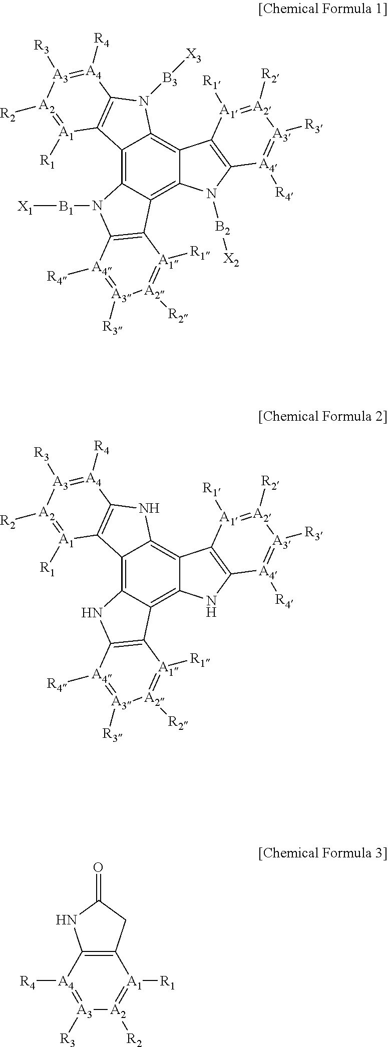

In [Chemical Formulas 1 to 3], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a sulfo group (—SO₃H).

Meanwhile, the perovskite solar cell of the present disclosure may include a laminate in which a hole transport layer, an organic interfacial layer, and a perovskite light-absorbing layer are sequentially stacked, and the organic interfacial layer may include a compound having an unshared electron pair for a perovskite solar cell represented by [Chemical Formula 1] below.

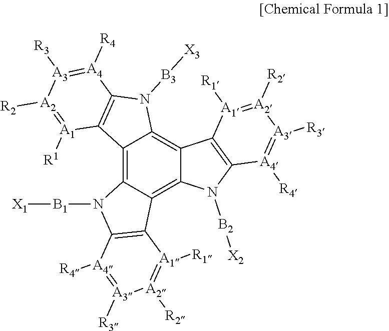

In [Chemical Formula 1], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a boronic acid group (—B(OH)₂), a boronic ester group, a dihydroxyphosphoryl group (—P═O(OH)₂), a trihalosilyl group (—SiX₃), a trialkoxysilyl group (—Si(OR)₃), a carboxyl group (—COOH), or a sulfo group (—SO₃H).

In a preferred embodiment of the present disclosure, the perovskite light-absorbing layer may include a perovskite material represented by [Chemical Formula 1] below.

CMX₃ [Chemical Formula 1]
wherein, in [Chemical Formula 1], C is a monovalent cation, M is a divalent cation, and X is a monovalent anion.

Meanwhile, a tandem perovskite solar cell of the present disclosure may include a laminate in which a solar cell, a recombination layer, a hole transport layer, an organic interfacial layer, and a perovskite light-absorbing layer are sequentially stacked, and the organic interfacial layer may include a compound having an unshared electron pair for a perovskite solar cell represented by [Chemical Formula 1] below.

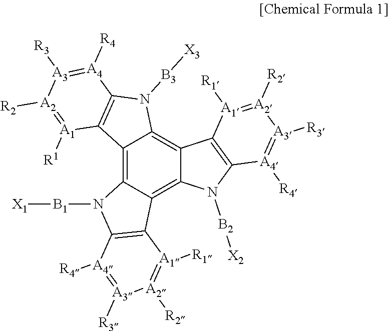

In [Chemical Formula 1], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently-H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a boronic acid group (—B(OH)₂), a boronic ester group, a dihydroxyphosphoryl group (—P═O(OH)₂), a trihalosilyl group (—SiX₃), a trialkoxysilyl group (—Si(OR)₃), a carboxyl group (—COOH), or a sulfo group (—SO₃H).

In a preferred embodiment of the present disclosure, the solar cell may be a polycrystalline silicon solar cell, a crystalline silicon solar cell, a perovskite solar cell, a gallium arsenide (GaAs) solar cell, a cadmium telluride (CdTe) solar cell, a CIGS (CuInGaSe) solar cell, a CZTS (Cu2_{z}nSnS₄) solar cell, an organic solar cell, a dye-sensitized solar cell, or a group III-V compound solar cell.

The compound having an unshared electron pair for a perovskite solar cell according to the present disclosure, a perovskite solar cell including the same, and a method for manufacturing the same can induce hydrogen bonding between molecules to improve intermolecular interaction.

In addition, the compound having an unshared electron pair for a perovskite solar cell according to the present disclosure, the perovskite solar cell including the same, and the method for manufacturing the same can secure an appropriate energy level for hole transport.

In addition, the compound having an unshared electron pair for a perovskite solar cell according to the present disclosure, the perovskite solar cell including the same, and the method for manufacturing the same can improve thin-film quality and prevent charge recombination.

### DETAILED DESCRIPTION OF THE EMBODIMENTS

Hereinafter, the present disclosure will be described in more detail.

In order for a perovskite solar cell to exhibit excellent efficiency, a material that induces hydrogen bonding between molecules to improve intermolecular interaction and possesses high hole-transport capability is required.

However, the materials conventionally used, even when placed at appropriate positions or in appropriate thin film layers in a perovskite solar cell, had the problem of not being able to achieve the highest efficiency.

Accordingly, the present disclosure relates to a compound having an unshared electron pair for a perovskite solar cell, which induces hydrogen bonding between molecules to improve intermolecular interaction and secures an energy level suitable for hole transport, a perovskite solar cell including the same, and a method of manufacturing the same.

The present disclosure includes a compound having an unshared electron pair for a perovskite solar cell, represented by [Chemical Formula 1] below.

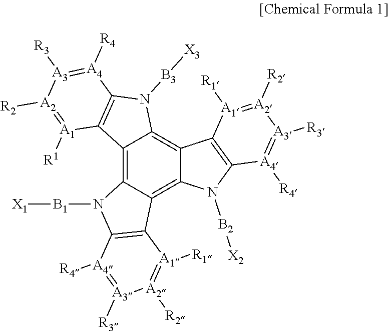

In [Chemical Formula 1], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N). However, at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N). For example, when A₂, A₃, A₄, A_{2′}, A_{3′}, A_{4′}, A_{2″}, A_{3″}, and A_{4″} are each carbon (C), A₁, A_{1′}, and A_{1″} are nitrogen (N).

In addition, in [Chemical Formula 1], R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group. However, when A₁, A₂, A₃, A₄, A_{1′}, A_{2′}, A_{3′}, A_{4′}, A_{1″}, A_{2″}, A_{3″}, and/or A_{4″} are nitrogen (N), R₁, R₂, R₃, R₄, R_{1′}, R_{2′}, R_{3′}, R_{4′}, R_{1″}, R_{2″}, R_{3″}, and/or R_{4″} bound to A₁, A₂, A₃, A₄, A_{1′}, A_{2′}, A_{3′}, A_{4′}, A_{1″}, A_{2″}, A_{3″}, and/or A_{4″} are unshared electron pairs.

In addition, in [Chemical Formula 1], B₁, B₂, and B₃ are each independently a single bond, a C6-C18 arylene group, or a C5-C18 heteroarylene group.

In addition, in [Chemical Formula 1], X₁, X₂, and X₃ are each independently a boronic acid group (—B(OH)₂), a boronic ester group, a dihydroxyphosphoryl group (—P═O(OH)₂), a trihalosilyl group (—SiX₃), a trialkoxysilyl group (—Si(OR)₃), a carboxyl group (—COOH), or a sulfo group (—SO₃H).

In addition, the compound having an unshared electron pair for a perovskite solar cell represented by [Chemical Formula 1] may be a compound having an unshared electron pair for a perovskite solar cell represented by [Chemical Formula 1-25] below.

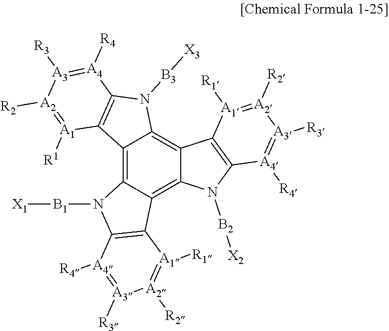

In [Chemical Formula 1-25], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N). However, at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N).

In addition, in [Chemical Formula 1-25], R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group. However, when A₁, A₂, A₃, A₄, A_{1′}, A_{2′}, A_{3′}, A_{4′}, A_{1″}, A_{2″}, A_{3″}, and/or A_{4″} are nitrogen (N), R₁, R₂, R₃, R₄, R_{1′}, R_{2′}, R_{3′}, R_{4′}, R_{1″}, R_{2″}, R_{3″}, and/or R_{4″} bound to A₁, A₂, A₃, A₄, A₁, A_{2′}, A_{3′}, A_{4′}, A_{1″}, A_{2″}, A_{3″}, and/or A_{4″} are unshared electron pairs.

In addition, in [Chemical Formula 1-25], B₁, B₂, and B₃ are each independently —CH₂—, —CH₂CH₂—, —CH₂CH₂CH₂—, —CH₂CH₂CH₂CH₂—, —CH₂CH₂CH₂CH₂CH₂—,

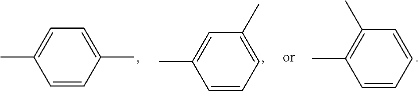

In addition, in [Chemical Formula 1-25], X₁, X₂, and X₃ are each independently a boronic acid group (—B(OH)₂),

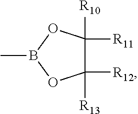

a dihydroxyphosphoryl group (—P═O(OH)₂), a carboxyl group (—COOH), or a sulfo group (—SO₃H).

In addition, in [Chemical Formula 1-25], R₁₀, R₁₁, R₁₂, and R¹³ are each independently —H, a C1-C20 linear alkyl group, or a C3-C20 branched alkyl group.

Meanwhile, the compound having an unshared electron pair for a perovskite solar cell of the present disclosure may include a reaction product of a third reaction product and hydrochloric acid (HCl). At this time, the third reaction product may be a reaction product of a second reaction product and trimethyl borate. In addition, the second reaction product may be a reaction product of a compound represented by [Chemical Formula 2] below, sodium hydride, and dibromobenzene.

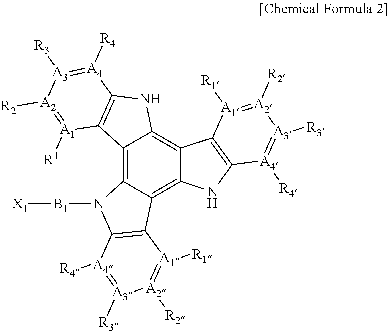

In [Chemical Formula 2], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N). However, at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N). For example, when A₂, A₃, A₄, A_{2′}, A_{3′}, A_{4′}, A_{2″}, A_{3″}, and A_{4″} are each carbon (C), A₁, A_{1′}, and A_{1″} are nitrogen (N).

In addition, in [Chemical Formula 2], R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group. However, when A₁, A₂, A₃, A₄, A_{1′}, A_{2′}, A_{3′}, A_{4′}, A_{1″}, A_{2″}, A_{3″}, and/or A_{4″} are nitrogen (N), R₁, R₂, R₃, R₄, R_{1′}, R_{2′}, R_{3′}, R_{4′}, R_{1″}, R_{2″}, R_{3″}, and/or R_{4″} bound to A₁, A₂, A₃, A₄, A_{1′}, A_{2′}, A_{3′}, A_{4′}, A_{1″}, A_{2″}, A_{3″}, and/or A_{4″} are unshared electron pairs.

In addition, the dibromobenzene may be 1,3-dibromobenzene or 1,4-dibromobenzene.

Furthermore, the compound having an unshared electron pair for a perovskite solar cell of the present disclosure may include a reaction product of the second reaction product, bis(pinacolato)diboron, Pd(dppf)Cl₂, and potassium acetate. At this time, the second reaction product may include a reaction product of a compound represented by [Chemical Formula 2], sodium hydride, and dibromobenzene.

Meanwhile, the compound having an unshared electron pair for a perovskite solar cell of the present disclosure may include a reaction product of the second reaction product and bromotrimethylsilane. At this time, the second reaction product may be a reaction product of the compound represented by [Chemical Formula 2], sodium hydride, and diethyl(3-bromopropyl)phosphonate.

Furthermore, the compound having an unshared electron pair for a perovskite solar cell of the present disclosure may include a reaction product of the third reaction product and hydrochloric acid (HCl). At this time, the third reaction product may be a reaction product of the second reaction product, diethyl phosphite, triphenylphosphine, and cesium carbonate. In addition, the second reaction product may be a reaction product of the compound represented by [Chemical Formula 2], sodium hydride, and 1,4-dibromobenzene.

Meanwhile, the compound having an unshared electron pair for a perovskite solar cell of the present disclosure may include a reaction product of the second reaction product and sodium hydroxide (NaOH). At this time, the second reaction product may be a reaction product of the compound represented by [Chemical Formula 2], ethyl 3-bromopropionate, potassium carbonate, and potassium iodide.

Furthermore, the compound having an unshared electron pair for a perovskite solar cell of the present disclosure may include a reaction product of the third reaction product and hydrochloric acid (HCl). At this time, the third reaction product may be a reaction product of the second reaction product and sodium sulfite. In addition, the second reaction product may be a reaction product of a compound represented by [Chemical Formula 2] below, 1,3-dibromoalkane, potassium carbonate, and potassium iodide. At this time, the 1,3-dibromoalkane may be 1,3-dibromopropane or 1,3-dibromobutane.

In addition, the compound represented by [Chemical Formula 2] may include a reaction product of a compound represented by [Chemical Formula 3] below and phosphoryl chloride.

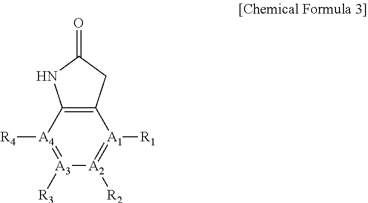

In [Chemical Formula 3], A₁, A₂, A₃, and A₄ are each independently carbon (C) or nitrogen (N). However, at least one of A₁, A₂, A₃, and A₄ is nitrogen (N).

In addition, in [Chemical Formula 3], R₁, R₂, R₃, and R₄ are each independently —H, a C1-C20 linear alkyl group, or a C3-C20 branched alkyl group. However, when A₁, A₂, A₃, and/or A₄ are nitrogen (N), R₁, R₂, R₃, and/or R₄ bound to A₁, A₂, A₃, and/or A₄ are unshared electron pairs.

Specifically, the compound having an unshared electron pair for a perovskite solar cell represented by [Chemical Formula 1] may be a compound having an unshared electron pair for a perovskite solar cell represented by [Chemical Formula 1-1] below.

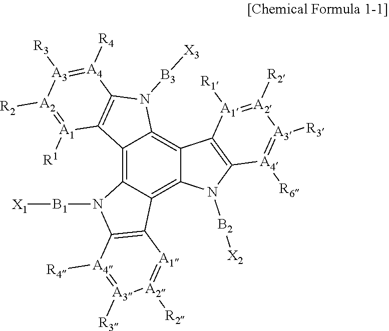

In [Chemical Formula 1-1], A₂, A₃, A₄, A_{2′}, A_{3′}, A_{4′}, A_{2″}, A_{3″}, and A_{4″} are each carbon (C), and A₁, A_{1′}, and A_{1″} are each nitrogen (N).

In [Chemical Formula 1-1], R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group.

In [Chemical Formula 1-1], B₁, B₂, and B₃ are each independently

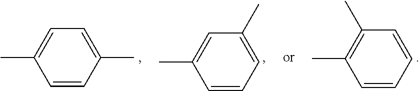

In [Chemical Formula 1-1], X₁, X₂, and X₃ are each a boronic acid group (—B(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-2] below.

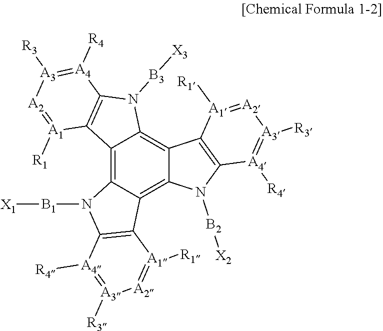

In [Chemical Formula 1-2], A₁, A₃, A₄, A_{1′}, A_{3′}, A_{4′}, A_{1″}, A_{3″}, and A_{4″} are each carbon (C), and A₂, A_{2′}, and A_{2″} are each nitrogen (N).

In addition, in [Chemical Formula 1-2], R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group.

In addition, in [Chemical Formula 1-2], B₁, B₂, and B₃ are each independently

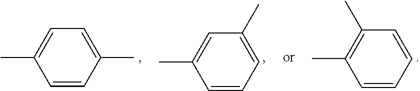

In addition, in [Chemical Formula 1-2], X₁, X₂, and X₃ are each a boronic acid group (—B(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-3] below.

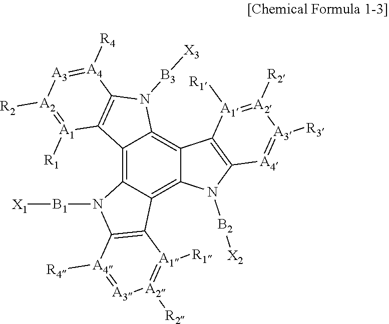

In [Chemical Formula 1-3], A₁, A₂, A₄, A_{1′}, A_{2′}, A_{4′}, A_{1″}, A_{2″}, and A_{4″} are each carbon (C), and A₃, A_{3′}, and A_{3″} are each nitrogen (N).

In addition, in [Chemical Formula 1-3], R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group.

In addition, in [Chemical Formula 1-3], B₁, B₂, and B₃ are each independently

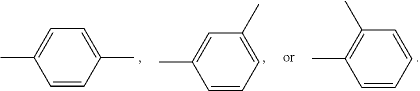

In addition, in [Chemical Formula 1-3], X₁, X₂, and X₃ are each a boronic acid group (—B(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-4] below.

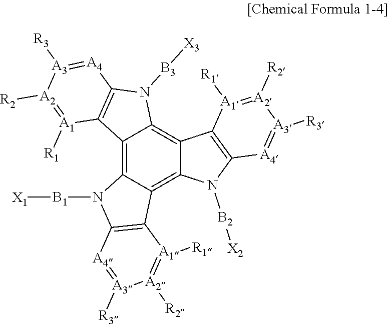

In [Chemical Formula 1-4], A₁, A₂, A₃, A_{1′}, A_{2′}, A_{3′}, A_{1″}, A_{2″}, and A_{3″} are each carbon (C), and A₄, A_{4′}, and A_{4″} are each nitrogen (N).

In addition, in [Chemical Formula 1-4], R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group.

In addition, in [Chemical Formula 1-4], B₁, B₂, and B₃ are each independently

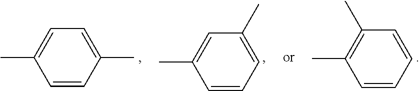

In addition, in [Chemical Formula 1-4], X₁, X₂, and X₃ are each a boronic acid group (—B(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-6] below.

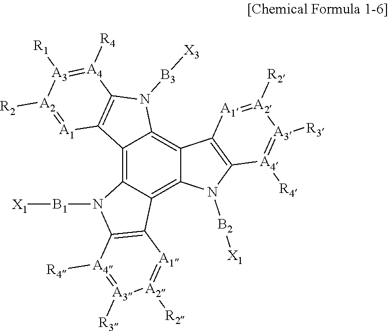

In [Chemical Formula 1-6], A₂, A₃, A₄, A_{2′}, A_{3′}, A_{4′}, A_{2″}, A_{3″}, and A_{4″} are each carbon (C), and A₁, A_{1′}, and A_{1″} are each nitrogen (N).

In addition, in [Chemical Formula 1-6], R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group.

In addition, in [Chemical Formula 1-6], B₁, B₂, and B₃ are each independently

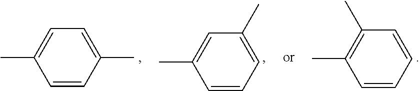

In addition, in [Chemical Formula 1-6], X₁, X₂, and X₃ are each

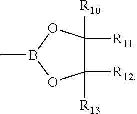

In addition, in [Chemical Formula 1-6], R₁₀, R₁₁, R₁₂, and R¹³ are each independently —H, a C1-C20 linear alkyl group, or a C3-C20 branched alkyl group.

In addition, specifically, the compound having an unshared electron pair for a perovskite solar cell represented by [Chemical Formula 1] may be a compound having an unshared electron pair for a perovskite solar cell represented by [Chemical Formula 1-7] below.

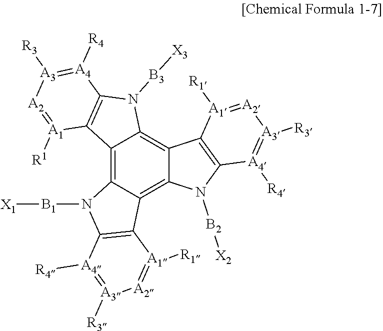

In [Chemical Formula 1-7], A₁, A₃, A₄, A_{1′}, A_{3′}, A_{4′}, A_{1″}, A_{3″}, and A_{4″} are each carbon (C), and A₂, A_{2′}, and A_{2″} are each nitrogen (N).

In addition, in [Chemical Formula 1-7], R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group.

In addition, in [Chemical Formula 1-7], B₁, B₂, and B₃ are each independently

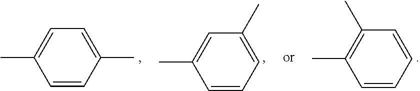

In addition, in [Chemical Formula 1-7], X₁, X₂, and X₃ are each

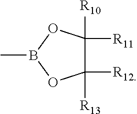

In addition, in [Chemical Formula 1-7], R₁₀, R₁₁, R₁₂, and R¹³ are each independently —H, a C1-C20 linear alkyl group, or a C3-C20 branched alkyl group.

In addition, specifically, the compound having an unshared electron pair for a perovskite solar cell represented by [Chemical Formula 1] may be a compound having an unshared electron pair for a perovskite solar cell represented by [Chemical Formula 1-8] below.

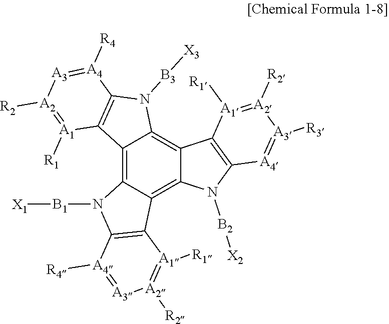

In [Chemical Formula 1-8], A₁, A₂, A₄, A_{1′}, A_{2′}, A_{4′}, A_{1″}, A_{2″}, and A_{4″} are each carbon (C), and A₃, A_{3′}, and A_{3″} are each nitrogen (N).

In addition, in [Chemical Formula 1-8], R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group.

In addition, in [Chemical Formula 1-8], B₁, B₂, and B₃ are each independently

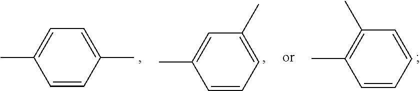

In addition, in [Chemical Formula 1-8], X₁, X₂, and X₃ are each

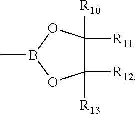

In addition, in [Chemical Formula 1-8], R₁₀, R₁₁, R₁₂, and R¹³ are each independently —H, a C1-C20 linear alkyl group, or a C3-C20 branched alkyl group.

In addition, specifically, the compound having an unshared electron pair for a perovskite solar cell represented by [Chemical Formula 1] may be a compound having an unshared electron pair for a perovskite solar cell represented by [Chemical Formula 1-9] below.

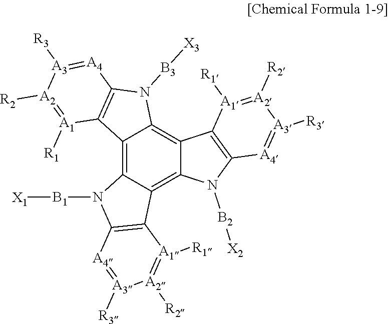

In [Chemical Formula 1-9], A₁, A₂, A₃, A_{1′}, A_{2′}, A_{3′}, A_{1″}, A_{2″}, and A_{3″} are each carbon (C), and A₄, A_{4′}, and A_{4″} are each nitrogen (N).

In addition, in [Chemical Formula 1-9], R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group.

In addition, in [Chemical Formula 1-9], B₁, B₂, and B₃ are each independently

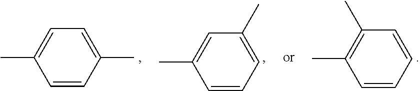

In addition, in [Chemical Formula 1-9], X₁, X₂, and X₃ are each

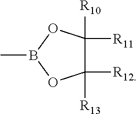

In addition, in [Chemical Formula 1-9], R₁₀, R₁₁, R₁₂, and R¹³ are each independently —H, a C1-C20 linear alkyl group, or a C3-C20 branched alkyl group.

In addition, specifically, the compound having an unshared electron pair for a perovskite solar cell represented by [Chemical Formula 1] may be a compound having an unshared electron pair for a perovskite solar cell represented by [Chemical Formula 1-10] below.

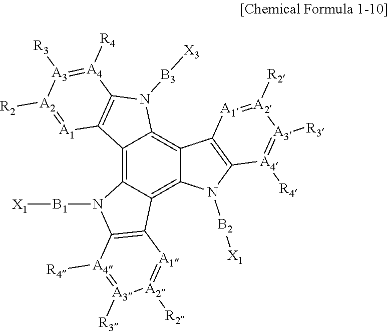

In [Chemical Formula 1-10], A₂, A₃, A₄, A_{2′}, A_{3′}, A_{4′}, A_{2″}, A_{3″}, and A_{4″} are each carbon (C), and A₁, A_{1′}, and A_{1″} are each nitrogen (N).

In [Chemical Formula 1-10], R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group.

In [Chemical Formula 1-10], B₁, B₂, and B₃ are each independently —CH₂CH₂—, —CH₂CH₂CH₂—, or —CH₂CH₂CH₂CH₂—.

In [Chemical Formula 1-10], X₁, X₂, and X₃ are each a dihydroxyphosphoryl group (—P═O(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-11] below.

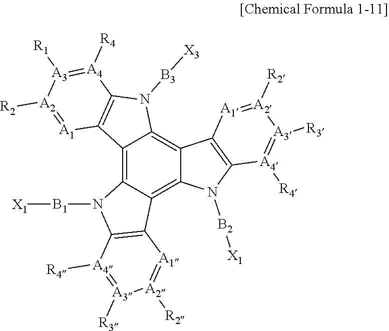

In [Chemical Formula 1-11], A₁, A₃, A₄, A_{1′}, A_{3′}, A_{4′}, A_{1″}, A_{3″}, and A_{4″} are each carbon (C), and A₂, A_{2′}, and A_{2″} are each nitrogen (N).

In addition, in [Chemical Formula 1-11], R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group.

In addition, in [Chemical Formula 1-11], B₁, B₂, and B₃ are each independently —CH₂CH₂—, —CH₂CH₂CH₂—, or —CH₂CH₂CH₂CH₂—.

In addition, in [Chemical Formula 1-11], X₁, X₂, and X₃ are each a dihydroxyphosphoryl group (—P═O(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-12] below.

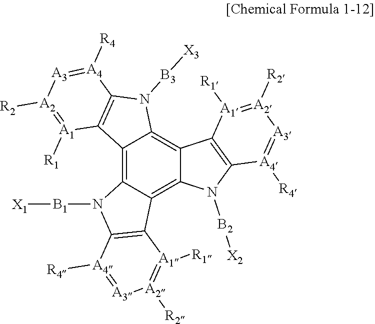

In [Chemical Formula 1-12], A₁, A₂, A₄, A_{1′}, A_{2′}, A_{4′}, A_{1″}, A_{2″}, and A_{4″} are each carbon (C), and A₃, A_{3′}, and A_{3″} are each nitrogen (N).

In addition, in [Chemical Formula 1-12], R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group.

In addition, in [Chemical Formula 1-12], B₁, B₂, and B₃ are each independently —CH₂CH₂—, —CH₂CH₂CH₂—, or —CH₂CH₂CH₂CH₂—.

In addition, in [Chemical Formula 1-12], X₁, X₂, and X₃ are each a dihydroxyphosphoryl group (—P═O(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-13] below.

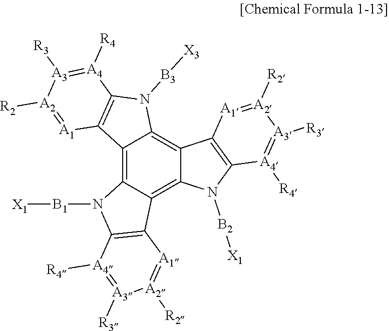

In [Chemical Formula 1-13], A₁, A₂, A₃, A_{1′}, A_{2′}, A_{3′}, A_{1″}, A_{2″}, and A_{3″} are each carbon (C), and A₄, A_{4′}, and A_{4″} are each nitrogen (N).

In addition, in [Chemical Formula 1-13], R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group.

In addition, in [Chemical Formula 1-13], B₁, B₂, and B₃ are each independently —CH₂CH₂—, —CH₂CH₂CH₂—, or —CH₂CH₂CH₂CH₂—.

In addition, in [Chemical Formula 1-13], X₁, X₂, and X₃ are each a dihydroxyphosphoryl group (—P═O(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-14] below.

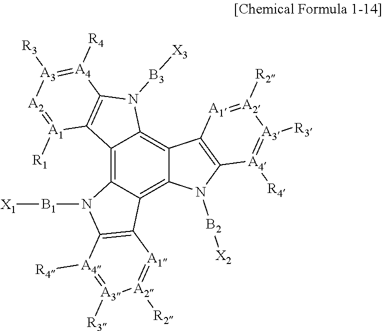

In [Chemical Formula 1-14], A₂, A₃, A₄, A_{2′}, A_{3′}, A_{4′}, A_{2″}, A_{3″}, and A_{4″} are each carbon (C), and A₁, A_{1′}, and A_{1″} are each nitrogen (N).

In [Chemical Formula 1-14], R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group.

In [Chemical Formula 1-14], B₁, B₂, and B₃ are each independently

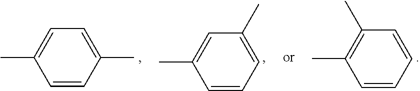

In [Chemical Formula 1-14], X₁, X₂, and X₃ are each a dihydroxyphosphoryl group (—P═O(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-15] below.

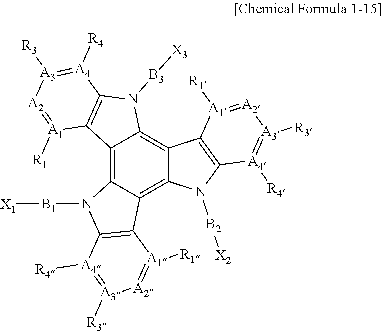

In [Chemical Formula 1-15], A₁, A₃, A₄, A_{1′}, A_{3′}, A_{4′}, A_{1″}, A_{3″}, and A_{4″} are each carbon (C), and A₂, A_{2′}, and A_{2″} are each nitrogen (N).

In addition, in [Chemical Formula 1-15], R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group.

In addition, in [Chemical Formula 1-15], B₁, B₂, and B₃ are each independently

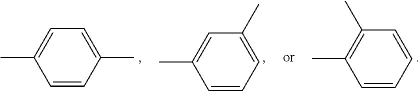

In addition, in [Chemical Formula 1-15], X₁, X₂, and X₃ are each a dihydroxyphosphoryl group (—P═O(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-16] below.

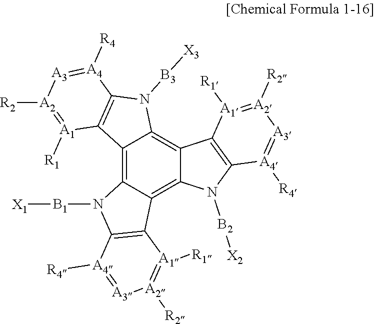

In [Chemical Formula 1-16], A₁, A₂, A₄, A_{1′}, A_{2′}, A_{4′}, A_{1″}, A_{2″}, and A_{4″} are each carbon (C), and A₃, A_{3′}, and A_{3″} are each nitrogen (N).

In addition, in [Chemical Formula 1-16], R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group.

In addition, in [Chemical Formula 1-16], B₁, B₂, and B₃ are each independently

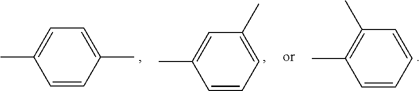

In addition, in [Chemical Formula 1-16], X₁, X₂, and X₃ are each a dihydroxyphosphoryl group (—P═O(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-17] below.

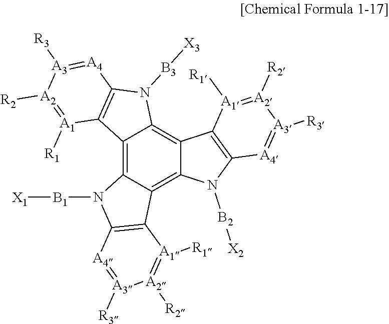

In [Chemical Formula 1-17], A₁, A₂, A₃, A_{1′}, A_{2′}, A_{3′}, A_{1″}, A_{2″}, and A_{3″} are each carbon (C), and A₄, A_{4′}, and A_{4″} are each nitrogen (N).

In addition, in [Chemical Formula 1-17], R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group.

In addition, in [Chemical Formula 1-17], B₁, B₂, and B₃ are each independently

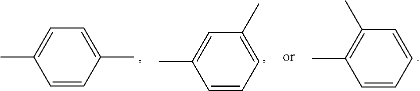

In addition, in [Chemical Formula 1-17], X₁, X₂, and X₃ are each a dihydroxyphosphoryl group (—P═O(OH)₂).

In addition, specifically, the compound having an unshared electron pair for a perovskite solar cell represented by [Chemical Formula 1] may be a compound having an unshared electron pair for a perovskite solar cell represented by [Chemical Formula 1-18] below.

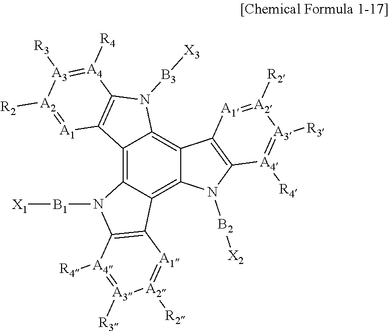

In [Chemical Formula 1-18], A₂, A₃, A₄, A_{2′}, A_{3′}, A_{4′}, A_{2″}, A_{3″}, and A_{4″} are each carbon (C), and A₁, A_{1′}, and A_{1″} are each nitrogen (N).

In [Chemical Formula 1-18], R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group.

In [Chemical Formula 1-18], B₁, B₂, and B₃ are each independently —CH₂—, —CH₂CH₂—, —CH₂CH₂CH₂—, or —CH₂CH₂CH₂CH₂—.

In [Chemical Formula 1-18], X₁, X₂, and X₃ are each a carboxyl group (—COOH).

In addition, specifically, the compound having an unshared electron pair for a perovskite solar cell represented by [Chemical Formula 1] may be a compound having an unshared electron pair for a perovskite solar cell represented by [Chemical Formula 1-19] below.

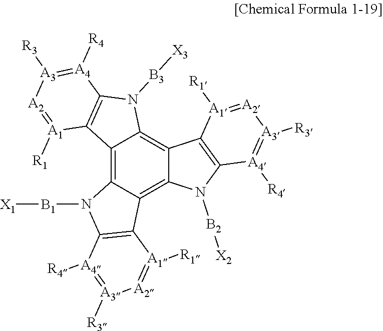

In [Chemical Formula 1-19], A₁, A₃, A₄, A_{1′}, A_{3′}, A_{4′}, A_{1″}, A_{3″}, and A_{4″} are each carbon (C), and A₂, A_{2′}, and A_{2″} are each nitrogen (N).

In addition, in [Chemical Formula 1-19], R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group.

In addition, in [Chemical Formula 1-19], B₁, B₂, and B₃ are each independently —CH₂—, —CH₂CH₂—, —CH₂CH₂CH₂—, or —CH₂CH₂CH₂CH₂—.

In addition, in [Chemical Formula 1-19], X₁, X₂, and X₃ are each a carboxyl group (—COOH).

In addition, specifically, the compound having an unshared electron pair for a perovskite solar cell represented by [Chemical Formula 1] may be a compound having an unshared electron pair for a perovskite solar cell represented by [Chemical Formula 1-20] below.

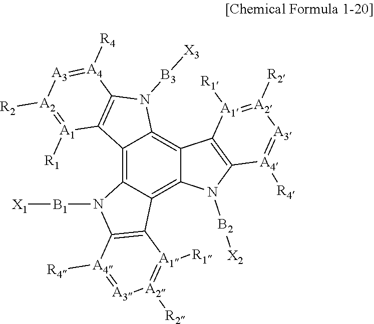

In [Chemical Formula 1-20], A₁, A₂, A₄, A_{1′}, A_{2′}, A_{4′}, A_{1″}, A_{2″}, and A_{4″} are each carbon (C), and A₃, A_{3′}, and A_{3″} are each nitrogen (N).

In addition, in [Chemical Formula 1-20], R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group.

In addition, in [Chemical Formula 1-20], B₁, B₂, and B₃ are each independently —CH₂—, —CH₂CH₂—, —CH₂CH₂CH₂—, or —CH₂CH₂CH₂CH₂—.

In addition, in [Chemical Formula 1-20], X₁, X₂, and X₃ are each a carboxyl group (—COOH).

In addition, specifically, the compound having an unshared electron pair for a perovskite solar cell represented by [Chemical Formula 1] may be a compound having an unshared electron pair for a perovskite solar cell represented by [Chemical Formula 1-26] below.

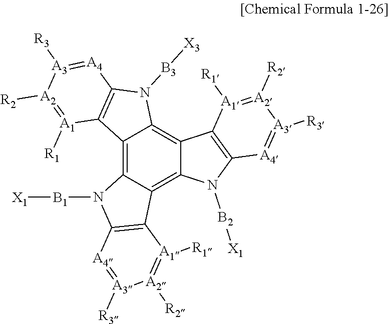

In [Chemical Formula 1-26], A₁, A₂, A₃, A_{1′}, A_{2′}, A_{3′}, A_{1″}, A_{2″}, and A_{3″} are each carbon (C), and A₄, A_{4′}, and A_{4″} are each nitrogen (N).

In addition, in [Chemical Formula 1-26], R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group.

In addition, in [Chemical Formula 1-26], B₁, B₂, and B₃ are each independently —CH₂—, —CH₂CH₂—, —CH₂CH₂CH₂—, or —CH₂CH₂CH₂CH₂—.

In addition, in [Chemical Formula 1-26], X₁, X₂, and X₃ are each a carboxyl group (—COOH).

In addition, specifically, the compound having an unshared electron pair for a perovskite solar cell represented by [Chemical Formula 1] may be a compound having an unshared electron pair for a perovskite solar cell represented by [Chemical Formula 1-21] below.

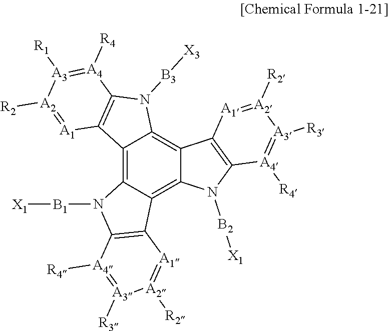

In [Chemical Formula 1-21], A₂, A₃, A₄, A_{2′}, A_{3′}, A_{4′}, A_{2″}, A_{3″}, and A_{4″} are each carbon (C), and A₁, A_{1′}, and A_{1″} are each nitrogen (N).

In [Chemical Formula 1-21], R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group.

In [Chemical Formula 1-21], B₁, B₂, and B₃ are each independently —CH₂—, —CH₂CH₂—, —CH₂CH₂CH₂—, or —CH₂CH₂CH₂CH₂—.

In [Chemical Formula 1-21], X₁, X₂, and X₃ are each a sulfo group (—SO₃H).

In addition, specifically, the compound having an unshared electron pair for a perovskite solar cell represented by [Chemical Formula 1] may be a compound having an unshared electron pair for a perovskite solar cell represented by [Chemical Formula 1-22] below.

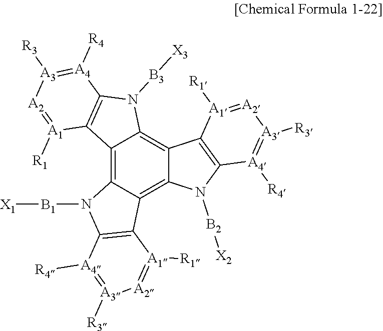

In [Chemical Formula 1-22], A₁, A₃, A₄, A_{1′}, A_{3′}, A_{4′}, A_{1″}, A_{3″}, and A_{4″} are each carbon (C), and A₂, A_{2′}, and A_{2″} are each nitrogen (N).

In addition, in [Chemical Formula 1-22], R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group.

In addition, in [Chemical Formula 1-22], B₁, B₂, and B₃ are each independently —CH₂—, —CH₂CH₂—, —CH₂CH₂CH₂—, or —CH₂CH₂CH₂CH₂—.

In addition, in [Chemical Formula 1-22], X₁, X₂, and X₃ are each a sulfo group (—SO₃H).

In addition, specifically, the compound having an unshared electron pair for a perovskite solar cell represented by [Chemical Formula 1] may be a compound having an unshared electron pair for a perovskite solar cell represented by [Chemical Formula 1-23] below.

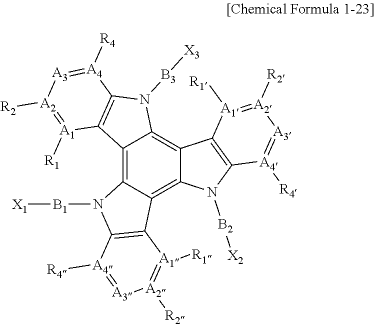

In [Chemical Formula 1-23], A₁, A₂, A₄, A_{1′}, A_{2′}, A_{4′}, A_{1″}, A_{2″}, and A_{4″} are each carbon (C), and A₃, A_{3′}, and A_{3″} are each nitrogen (N).

In addition, in [Chemical Formula 1-23], R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group.

In addition, in [Chemical Formula 1-23], B₁, B₂, and B₃ are each independently —CH₂—, —CH₂CH₂—, —CH₂CH₂CH₂—, or —CH₂CH₂CH₂CH₂—.

In addition, in [Chemical Formula 1-23], X₁, X₂, and X₃ are each a sulfo group (—SO₃H).

In addition, specifically, the compound having an unshared electron pair for a perovskite solar cell represented by [Chemical Formula 1] may be a compound having an unshared electron pair for a perovskite solar cell represented by [Chemical Formula 1-27] below.

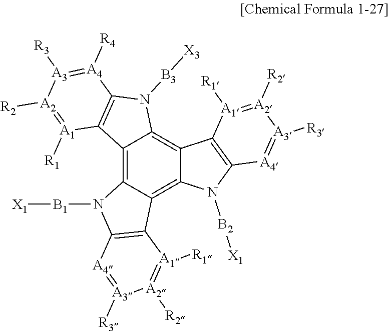

In [Chemical Formula 1-27], A₁, A₂, A₃, A_{1′}, A_{2′}, A_{3′}, A_{1″}, A_{2″}, and A_{3″} are each carbon (C), and A₄, A_{4′}, and A_{4″} are each nitrogen (N).

In addition, in [Chemical Formula 1-27], R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group.

In addition, in [Chemical Formula 1-27], B₁, B₂, and B₃ are each independently —CH₂—, —CH₂CH₂—, —CH₂CH₂CH₂—, or —CH₂CH₂CH₂CH₂—.

In addition, in [Chemical Formula 1-27], X₁, X₂, and X₃ are each a sulfo group (—SO₃H).

Meanwhile, a method for manufacturing the compound having an unshared electron pair for a perovskite solar cell of the present disclosure includes steps 1 to 4.

Step 1 of the method for manufacturing the compound having an unshared electron pair for a perovskite solar cell of the present disclosure may include feeding the compound represented by [Chemical Formula 3] to phosphoryl chloride and reacting them to produce the compound represented by [Chemical Formula 2].

Specifically, step 1 of the method for manufacturing the compound having an unshared electron pair for a perovskite solar cell of the present disclosure may include steps 1-1 to 1-3.

Step 1-1 of the method for manufacturing the compound having an unshared electron pair for a perovskite solar cell of the present disclosure may include feeding the compound represented by [Chemical Formula 3] to phosphoryl chloride, and then refluxing with stirring at a temperature of 130 to 170° C., preferably 140 to 160° C., for 12 to 36 hours, preferably 18 to 30 hours, and more preferably 20 to 26 hours, to produce a first reaction product. At this time, when the reaction temperature is lower than 130° C., there may be a problem of reduced synthesis yield due to the presence of unreacted products, and when the temperature exceeds 170° C., the synthesis yield may decrease due to side reactions and, in addition, purification may become impossible.

Step 1-2 of the method for manufacturing the compound having an unshared electron pair for a perovskite solar cell of the present disclosure may include neutralizing the first reaction product prepared in step 1-1 with a neutralizing agent, filtering a precipitate generated thereby to obtain a filtrate, and purifying the obtained filtrate to produce a purified product.

At this time, the neutralizing agent may include at least one selected from sodium hydroxide, sodium carbonate, potassium hydroxide, ammonium hydroxide, and sodium bicarbonate, and preferably may include sodium hydroxide. In addition, the purification may be performed through silica gel column chromatography.

Step 1-3 of the method for manufacturing the compound having an unshared electron pair for a perovskite solar cell of the present disclosure may include concentrating the purified product prepared in step 1-2 under reduced pressure and then recrystallizing to produce the compound represented by [Chemical Formula 2].

At this time, the recrystallization may be performed using acetone, methanol, ethanol, isopropyl alcohol, or a mixture thereof, and may preferably be performed using acetone.

Step 2 of the method for manufacturing the compound having an unshared electron pair for a perovskite solar cell of the present disclosure may include feeding sodium hydride and dibromobenzene to the compound represented by [Chemical Formula 2] and reacting them to produce a second reaction product. At this time, the dibromobenzene may be 1,3-dibromobenzene or 1,4-dibromobenzene.

Specifically, step 2 of the method for manufacturing the compound having an unshared electron pair for a perovskite solar cell of the present disclosure may include steps 2-1 to 2-2.

Step 2-1 of the method for manufacturing the compound having an unshared electron pair for a perovskite solar cell of the present disclosure may include dissolving the compound represented by [Chemical Formula 2] in an organic solvent, feeding sodium hydride, and stirring at a temperature of 15 to 35° C., preferably 20 to 30° C., for 1 to 30 minutes, preferably 5 to 15 minutes, to produce a mixture.

At this time, when the stirring temperature is lower than 15° C., there may be a problem of a reduced reaction rate, and when it exceeds 35° C., there may be a problem of an increase in side reaction products. In addition, the organic solvent may include at least one selected from anhydrous dimethylformamide, dimethylacetamide, and dimethyl sulfoxide, and may preferably include anhydrous dimethylformamide.

In addition, the compound represented by [Chemical Formula 2] and sodium hydride may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 2-2 of the method for manufacturing the compound having an unshared electron pair for a perovskite solar cell of the present disclosure may include feeding dibromobenzene to the mixture prepared in step 2-1 and stirring at a temperature of 80 to 120° C., preferably 90 to 110° C., for 5 to 15 hours, preferably 8 to 12 hours, to produce a second reaction product. At this time, when the reaction temperature is lower than 80° C., there may be a problem of reduced synthesis yield due to the presence of unreacted products, and when it exceeds 120° C., the synthesis yield may decrease due to side reactions and, in addition, purification may become impossible.

In addition, in step 2-2, the compound represented by [Chemical Formula 2] and dibromobenzene may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 3 of the method for manufacturing the compound having an unshared electron pair for a perovskite solar cell of the present disclosure may include feeding trimethyl borate to the second reaction product prepared in step 2 and reacting them to produce a third reaction product.

Specifically, step 3 of the method for manufacturing the compound having an unshared electron pair for a perovskite solar cell of the present disclosure may include steps 3-1 and 3-2.

Step 3-1 of the method for manufacturing the compound having an unshared electron pair for a perovskite solar cell of the present disclosure may include extracting the second reaction product prepared in step 2 with an organic solvent, concentrating the organic phase of the extract under reduced pressure to obtain a concentrate, purifying the concentrate, and drying to obtain a dried product. At this time, the organic solvent may include at least one selected from dichloromethane, chloroform, and tetrahydrofuran, and may preferably include dichloromethane. In addition, the purification may be performed through silica gel chromatography.

Step 3-2 of the method for manufacturing the compound having an unshared electron pair for a perovskite solar cell of the present disclosure may include dissolving the dried product obtained in step 3-1 in an organic solvent, dropwise adding n-butyllithium, and then dropwise adding trimethyl borate, followed by stirring and reacting at a temperature of 15 to 35° C., preferably 20 to 30° C., for 6 to 18 hours, preferably 10 to 14 hours, to produce a third reaction product. At this time, the organic solvent may include at least one selected from dichloromethane, chloroform, and tetrahydrofuran, and may preferably include anhydrous tetrahydrofuran having a temperature of −5 to 5° C., more preferably 0 to 3° C.

In addition, when the reaction temperature is lower than 15° C., there may be a problem of reduced synthesis yield due to an increase in unreacted products, and when it exceeds 35° C., the synthesis yield may decrease due to side reactions and, in addition, purification may become impossible.

In addition, in step 3-2, the compound represented by [Chemical Formula 2] and trimethyl borate may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 4 of the method for manufacturing the compound having an unshared electron pair for a perovskite solar cell of the present disclosure may include feeding hydrochloric acid (HCl) to the third reaction product prepared in step 3 and reacting them to produce the compound having an unshared electron pair for a perovskite solar cell represented by [Chemical Formula 1].

Specifically, step 4 of the method for manufacturing the compound having an unshared electron pair for a perovskite solar cell of the present disclosure may include steps 4-1 and 4-2.

Step 4-1 of the method for manufacturing the compound having an unshared electron pair for a perovskite solar cell of the present disclosure may include feeding hydrochloric acid (HCl) to the third reaction product prepared in step 3 and stirring to produce a fourth reaction product. At this time, the hydrochloric acid (HCl) may be a 0.5 to 2 N hydrochloric acid solution, but is not limited thereto.

In addition, in step 4-1, hydrochloric acid (HCl) may be fed such that the compound represented by [Chemical Formula 2] and hydrochloric acid (HCl) are in a molar ratio of 1:5 to 1:10, preferably 1:6 to 1:9, and more preferably 1:7 to 1:8. If the molar ratio is less than 1:6, there may be a problem of reduced synthesis yield due to an increase in unreacted products, and if it exceeds 1:9, there may be a problem of an increase in side reaction products.

Step 4-2 of the method for manufacturing the compound having an unshared electron pair for a perovskite solar cell of the present disclosure may include separating the organic phase of the fourth reaction product prepared in step 4-1, concentrating it under reduced pressure, and purifying it to produce the compound having an unshared electron pair for a perovskite solar cell represented by [Chemical Formula 1]. At this time, the purification may be performed through silica gel chromatography.

Furthermore, another method for manufacturing the compound having an unshared electron pair for a perovskite solar cell of the present disclosure includes steps 1 to 3.

Step 1 of another method for manufacturing the compound having an unshared electron pair for a perovskite solar cell of the present disclosure may include feeding the compound represented by [Chemical Formula 3] to phosphoryl chloride and reacting them to produce the compound represented by [Chemical Formula 2].

Specifically, step 1 of another method for manufacturing the compound having an unshared electron pair for a perovskite solar cell of the present disclosure may include steps 1-1 to 1-3.

Step 1-1 of another method for manufacturing the compound having an unshared electron pair for a perovskite solar cell of the present disclosure may include feeding the compound represented by [Chemical Formula 3] to phosphoryl chloride, and then refluxing with stirring at a temperature of 130 to 170° C., preferably 140 to 160° C., for 12 to 36 hours, preferably 18 to 30 hours, and more preferably 20 to 26 hours, to produce a first reaction product. At this time, when the reaction temperature is lower than 130° C., there may be a problem of reduced synthesis yield due to the presence of unreacted products, and when the temperature exceeds 170° C., the synthesis yield may decrease due to side reactions and, in addition, purification may become impossible.

Step 1-2 of another method for manufacturing the compound having an unshared electron pair for a perovskite solar cell of the present disclosure may include neutralizing the first reaction product prepared in step 1-1 with a neutralizing agent, filtering a precipitate generated thereby to obtain a filtrate, and purifying the obtained filtrate to produce a purified product.

At this time, the neutralizing agent may include at least one selected from sodium hydroxide, sodium carbonate, potassium hydroxide, ammonium hydroxide, and sodium bicarbonate, and preferably may include sodium hydroxide. In addition, the purification may be performed through silica gel column chromatography.

Step 1-3 of another method for manufacturing the compound having an unshared electron pair for a perovskite solar cell of the present disclosure may include concentrating the purified product prepared in step 1-2 under reduced pressure and then recrystallizing to produce the compound represented by [Chemical Formula 2].

At this time, the recrystallization may be performed using acetone, methanol, ethanol, isopropyl alcohol, or a mixture thereof, and may preferably be performed using acetone.

Step 2 of another method for manufacturing the compound having an unshared electron pair for a perovskite solar cell of the present disclosure may include feeding sodium hydride and dibromobenzene to the compound represented by [Chemical Formula 2] and reacting them to produce a second reaction product. At this time, the dibromobenzene may be 1,3-dibromobenzene or 1,4-dibromobenzene.

Specifically, step 2 of another method for manufacturing the compound having an unshared electron pair for a perovskite solar cell of the present disclosure may include steps 2-1 to 2-2.

Step 2-1 of another method for manufacturing the compound having an unshared electron pair for a perovskite solar cell of the present disclosure may include dissolving the compound represented by [Chemical Formula 2] in an organic solvent, feeding sodium hydride, and stirring at a temperature of 15 to 35° C., preferably 20 to 30° C., for 1 to 30 minutes, preferably 5 to 15 minutes, to produce a mixture.

At this time, when the stirring temperature is lower than 15° C., there may be a problem of a reduced reaction rate, and when it exceeds 35° C., there may be a problem of an increase in side reaction products. In addition, the organic solvent may include at least one selected from anhydrous dimethylformamide, dimethylacetamide, and dimethyl sulfoxide, and may preferably include anhydrous dimethylformamide.

In addition, the compound represented by [Chemical Formula 2] and sodium hydride may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 2-2 of another method for manufacturing the compound having an unshared electron pair for a perovskite solar cell of the present disclosure may include feeding dibromobenzene to the mixture prepared in step 2-1 and stirring at a temperature of 80 to 120° C., preferably 90 to 110° C., for 5 to 15 hours, preferably 8 to 12 hours, to produce a second reaction product. At this time, when the reaction temperature is lower than 80° C., there may be a problem of reduced synthesis yield due to the presence of unreacted products, and when it exceeds 120° C., the synthesis yield may decrease due to side reactions and, in addition, purification may become impossible.

In addition, in step 2-2, the compound represented by [Chemical Formula 2] and dibromobenzene may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 3 of another method for manufacturing the compound having an unshared electron pair for a perovskite solar cell of the present disclosure may include feeding bis(pinacolato)diboron, Pd(dppf)Cl₂, and potassium acetate to the second reaction product prepared in step 2 and reacting them to produce the compound having an unshared electron pair for a perovskite solar cell represented by [Chemical Formula 1].

Specifically, step 3 of another method for manufacturing a compound having an unshared electron pair for a perovskite solar cell of the present disclosure may include steps 3-1 to 3-3.

Step 3-1 of another method for manufacturing the compound having an unshared electron pair for a perovskite solar cell of the present disclosure may include extracting the second reaction product prepared in step 2 with an organic solvent, concentrating the organic phase of the extract under reduced pressure to obtain a concentrate, purifying the concentrate, and drying to obtain a dried product. At this time, the organic solvent may include at least one selected from dichloromethane, chloroform, and tetrahydrofuran, and may preferably include dichloromethane. In addition, the purification may be performed through silica gel chromatography.

Step 3-2 of another method for manufacturing the compound having an unshared electron pair for a perovskite solar cell of the present disclosure may include dissolving the dried product obtained in step 3-1 in an organic solvent, feeding bis(pinacolato)diboron, Pd(dppf)Cl₂, and potassium acetate, and stirring and reacting at a temperature of 80 to 120° C., preferably 90 to 110° C., for 5 to 15 hours, preferably 8 to 12 hours, to produce a third reaction product. At this time, the organic material may be 1,4-dioxane, but is not limited thereto. In addition, when the reaction temperature is lower than 80° C., there may be a problem of reduced synthesis yield due to an increase in unreacted products, and when it exceeds 120° C., the synthesis yield may decrease due to side reactions and, in addition, purification may become impossible. In addition, in step 3-2, the compound represented by [Chemical Formula 2] and bis(pinacolato)diboron may be mixed at a molar ratio of 1:5 to 1:9, preferably 1:6 to 1:8, and more preferably 1:6.5 to 1:7.5. If the molar ratio is less than 1:5, there may be a problem of reduced synthesis yield due to an increase in unreacted products, and if it exceeds 1:9, there may be a problem of an increase in side reaction products. In addition, in step 3-2, the compound represented by [Chemical Formula 2] and Pd(dppf)Cl₂ may be mixed at a molar ratio of 1:0.01 to 1:0.2, preferably 1:0.03 to 1:0.15, and more preferably 1:0.05 to 1:0.1. If the molar ratio is less than 1:0.01, there may be a problem of reduced synthesis yield due to an increase in unreacted products, and if it exceeds 1:0.2, there may be a problem of an increase in side reaction products. In addition, in step 3-2, the compound represented by [Chemical Formula 2] and potassium acetate may be mixed at a molar ratio of 1:4 to 1:8, preferably 1:5 to 1:7, and more preferably 1:5.5 to 1:6.5. If the molar ratio is less than 1:4, there may be a problem of reduced synthesis yield due to an increase in unreacted products, and if it exceeds 1:8, there may be a problem of an increase in side reaction products.

Step 3-3 of another method for manufacturing the compound having an unshared electron pair for a perovskite solar cell of the present disclosure may include extracting the third reaction product prepared in step 3-2 with an organic solvent, concentrating the organic phase of the extract under reduced pressure to obtain a concentrate, and purifying the concentrate to produce the compound having an unshared electron pair for a perovskite solar cell represented by [Chemical Formula 1]. At this time, the organic solvent may include at least one selected from dichloromethane, chloroform, and tetrahydrofuran, and may preferably include dichloromethane. In addition, the purification may be performed through silica gel chromatography.

Meanwhile, still another method for manufacturing the compound having an unshared electron pair for a perovskite solar cell of the present disclosure includes steps 1 to 3.

Step 1 of still another method for manufacturing the compound having an unshared electron pair for a perovskite solar cell of the present disclosure may include feeding the compound represented by [Chemical Formula 3] to phosphoryl chloride and reacting them to produce the compound represented by [Chemical Formula 2].

Specifically, step 1 of still another method for manufacturing the compound having an unshared electron pair for a perovskite solar cell of the present disclosure may include steps 1-1 to 1-3.

Step 1-1 of still another method for manufacturing the compound having an unshared electron pair for a perovskite solar cell of the present disclosure may include feeding the compound represented by

[Chemical Formula 3] to phosphoryl chloride, and then refluxing with stirring at a temperature of 130 to 170° C., preferably 140 to 160° C., for 12 to 36 hours, preferably 18 to 30 hours, and more preferably 20 to 26 hours, to produce a first reaction product. At this time, when the reaction temperature is lower than 130° C., there may be a problem of reduced synthesis yield due to the presence of unreacted products, and when the temperature exceeds 170° C., the synthesis yield may decrease due to side reactions and, in addition, purification may become impossible.

Step 1-2 of still another method for manufacturing the compound having an unshared electron pair for a perovskite solar cell of the present disclosure may include neutralizing the first reaction product prepared in step 1-1 with a neutralizing agent, filtering a precipitate generated thereby to obtain a filtrate, and purifying the obtained filtrate to produce a purified product.

At this time, the neutralizing agent may include at least one selected from sodium hydroxide, sodium carbonate, potassium hydroxide, ammonium hydroxide, and sodium bicarbonate, and preferably may include sodium hydroxide. In addition, the purification may be performed through silica gel column chromatography.

Step 1-3 of still another method for manufacturing the compound having an unshared electron pair for a perovskite solar cell of the present disclosure may include concentrating the purified product prepared in step 1-2 under reduced pressure and then recrystallizing to produce the compound represented by [Chemical Formula 2].

At this time, the recrystallization may be performed using acetone, methanol, ethanol, isopropyl alcohol, or a mixture thereof, and may preferably be performed using acetone.

Step 2 of still another method for manufacturing the compound having an unshared electron pair for a perovskite solar cell of the present disclosure may include feeding sodium hydride and diethyl(3-bromopropyl)phosphonate to the compound represented by [Chemical Formula 2] and reacting them to produce a second reaction product.

Specifically, step 2 of still another method for manufacturing the compound having an unshared electron pair for a perovskite solar cell of the present disclosure may include steps 2-1 to 2-2.

Step 2-1 of still another method for manufacturing the compound having an unshared electron pair for a perovskite solar cell of the present disclosure may include dissolving the compound represented by [Chemical Formula 2] in an organic solvent, feeding sodium hydride, and stirring at a temperature of 10 to 30° C., preferably 15 to 25° C., for 1 to 30 minutes, preferably 5 to 15 minutes, to produce a mixture.

At this time, when the stirring temperature is lower than 10° C., there may be a problem of a reduced reaction rate, and when it exceeds 30° C., there may be a problem of an increase in side reaction products. In addition, the organic solvent may include at least one selected from anhydrous dimethylformamide, dimethylacetamide, and dimethyl sulfoxide, and may preferably include anhydrous dimethylformamide.

In addition, the compound represented by [Chemical Formula 2] and sodium hydride may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 2-2 of still another method for manufacturing the compound having an unshared electron pair for a perovskite solar cell of the present disclosure may include feeding diethyl(3-bromopropyl)phosphonate to the mixture prepared in step 2-1 and stirring at a temperature of 80 to 120° C., preferably 90 to 110° C., for 5 to 15 hours, preferably 8 to 12 hours, to produce a second reaction product. At this time, when the reaction temperature is lower than 80° C., there may be a problem of reduced synthesis yield due to the presence of unreacted products, and when it exceeds 120° C., the synthesis yield may decrease due to side reactions and, in addition, purification may become impossible.

In addition, in step 2-2, the compound represented by [Chemical Formula 2] and diethyl(3-bromopropyl)phosphonate may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 3 of still another method for manufacturing the compound having an unshared electron pair for a perovskite solar cell of the present disclosure may include feeding bromotrimethylsilane to the second reaction product prepared in step 2 and reacting them to produce the compound having an unshared electron pair for a perovskite solar cell represented by [Chemical Formula 1].

Specifically, step 3 of still another method for manufacturing the compound having an unshared electron pair for a perovskite solar cell of the present disclosure may include steps 3-1 and 3-2.

Step 3-1 of still another method for manufacturing the compound having an unshared electron pair for a perovskite solar cell of the present disclosure may include extracting the second reaction product prepared in step 2 with an organic solvent, concentrating the organic phase of the extract under reduced pressure to obtain a concentrate, purifying the concentrate, and drying to obtain a dried product. At this time, the organic solvent may include at least one selected from dichloromethane, chloroform, and tetrahydrofuran, and may preferably include dichloromethane. In addition, the purification may be performed through silica gel chromatography.

Step 3-2 of still another method for manufacturing the compound having an unshared electron pair for a perovskite solar cell of the present disclosure may include dissolving the dried product obtained in step 3-1 in an organic solvent, feeding bromotrimethylsilane, and stirring and reacting at a temperature of 10 to 30° C., preferably 15 to 25° C., for 12 to 36 hours, preferably 20 to 28 hours, to produce a third reaction product, and recrystallizing the third reaction product to produce the compound having an unshared electron pair for a perovskite solar cell represented by [Chemical Formula 1].

At this time, when the reaction temperature is lower than 15° C., there may be a problem of reduced synthesis yield due to an increase in unreacted products, and when it exceeds 25° C., the synthesis yield may decrease due to side reactions and, in addition, purification may become impossible. In addition, the recrystallization may be performed using methanol, ethanol, isopropyl alcohol, or a mixture thereof, and may be preferably performed using methanol.

In addition, in step 3-2, the compound represented by [Chemical Formula 2] and bromotrimethylsilane may be mixed at a molar ratio of 1:3 to 1:7, preferably 1:4 to 1:6, and more preferably 1:4.5 to 1:5.5. If the molar ratio is less than 1:3, there may be a problem of reduced synthesis yield due to an increase in unreacted products, and if it exceeds 1:7, there may be a problem of an increase in side reaction products.

Furthermore, still another method for manufacturing the compound having an unshared electron pair for a perovskite solar cell of the present disclosure includes steps 1 to 4.

Step 1 of still another method for manufacturing the compound having an unshared electron pair for a perovskite solar cell of the present disclosure may include feeding the compound represented by [Chemical Formula 3] to phosphoryl chloride and reacting them to produce the compound represented by [Chemical Formula 2].

Specifically, step 1 of still another method for manufacturing the compound having an unshared electron pair for a perovskite solar cell of the present disclosure may include steps 1-1 to 1-3.

Step 1-1 of still another method for manufacturing the compound having an unshared electron pair for a perovskite solar cell of the present disclosure may include feeding the compound represented by [Chemical Formula 3] to phosphoryl chloride, and then refluxing with stirring at a temperature of 130 to 170° C., preferably 140 to 160° C., for 12 to 36 hours, preferably 18 to 30 hours, and more preferably 20 to 26 hours, to produce a first reaction product. At this time, when the reaction temperature is lower than 130° C., there may be a problem of reduced synthesis yield due to the presence of unreacted products, and when the temperature exceeds 170° C., the synthesis yield may decrease due to side reactions and, in addition, purification may become impossible.

Step 1-2 of still another method for manufacturing the compound having an unshared electron pair for a perovskite solar cell of the present disclosure may include neutralizing the first reaction product prepared in step 1-1 with a neutralizing agent, filtering a precipitate generated thereby to obtain a filtrate, and purifying the obtained filtrate to produce a purified product.

At this time, the neutralizing agent may include at least one selected from sodium hydroxide, sodium carbonate, potassium hydroxide, ammonium hydroxide, and sodium bicarbonate, and preferably may include sodium hydroxide. In addition, the purification may be performed through silica gel column chromatography.

Step 1-3 of still another method for manufacturing the compound having an unshared electron pair for a perovskite solar cell of the present disclosure may include concentrating the purified product prepared in step 1-2 under reduced pressure and then recrystallizing to produce the compound represented by [Chemical Formula 2].

At this time, the recrystallization may be performed using acetone, methanol, ethanol, isopropyl alcohol, or a mixture thereof, and may preferably be performed using acetone.

Step 2 of still another method for manufacturing the compound having an unshared electron pair for a perovskite solar cell of the present disclosure may include feeding sodium hydride and 1,4-dibromobenzene to the compound represented by [Chemical Formula 2] and reacting them to produce a second reaction product.

Specifically, step 2 of still another method for manufacturing the compound having an unshared electron pair for a perovskite solar cell of the present disclosure may include steps 2-1 to 2-2.

Step 2-1 of still another method for manufacturing the compound having an unshared electron pair for a perovskite solar cell of the present disclosure may include dissolving the compound represented by [Chemical Formula 2] in an organic solvent, feeding sodium hydride, and stirring at a temperature of 15 to 35° C., preferably 20 to 30° C., for 1 to 30 minutes, preferably 5 to 15 minutes, to produce a mixture.

At this time, when the stirring temperature is lower than 15° C., there may be a problem of a reduced reaction rate, and when it exceeds 35° C., there may be a problem of an increase in side reaction products. In addition, the organic solvent may include at least one selected from anhydrous dimethylformamide, dimethylacetamide, and dimethyl sulfoxide, and may preferably include anhydrous dimethylformamide.

In addition, the compound represented by [Chemical Formula 2] and sodium hydride may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 2-2 of still another method for manufacturing the compound having an unshared electron pair for a perovskite solar cell of the present disclosure may include feeding 1,4-dibromobenzene to the mixture prepared in step 2-1 and stirring at a temperature of 80 to 120° C., preferably 90 to 110° C., for 5 to 15 hours, preferably 8 to 12 hours, to produce a second reaction product. At this time, when the reaction temperature is lower than 80° C., there may be a problem of reduced synthesis yield due to the presence of unreacted products, and when it exceeds 120° C., the synthesis yield may decrease due to side reactions and, in addition, purification may become impossible.

In addition, in step 2-2, the compound represented by [Chemical Formula 2] and 1,4-dibromobenzene may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 3 of still another method for manufacturing the compound having an unshared electron pair for a perovskite solar cell of the present disclosure may include feeding diethyl phosphite, triphenylphosphine, and cesium carbonate to the second reaction product prepared in step 2 and reacting them to produce a third reaction product.

Specifically, step 3 of still another method for manufacturing the compound having an unshared electron pair for a perovskite solar cell of the present disclosure may include steps 3-1 and 3-2.

Step 3-1 of still another method for manufacturing the compound having an unshared electron pair for a perovskite solar cell of the present disclosure may include extracting the second reaction product prepared in step 2 with an organic solvent, concentrating the organic phase of the extract under reduced pressure to obtain a concentrate, purifying the concentrate, and drying to obtain a dried product. At this time, the organic solvent may include at least one selected from dichloromethane, chloroform, and tetrahydrofuran, and may preferably include dichloromethane. In addition, the purification may be performed through silica gel chromatography.

Step 3-2 of still another method for manufacturing the compound having an unshared electron pair for a perovskite solar cell of the present disclosure may include dissolving the dried product obtained in step 3-1 in an organic solvent, feeding diethyl phosphite, triphenylphosphine, and cesium carbonate, and stirring and reacting at a temperature of 90 to 130° C., preferably 100 to 120° C., for 6 to 18 hours, preferably 9 to 15 hours, to produce a third reaction product. At this time, the organic material may be anhydrous toluene, but is not limited thereto. In addition, when the reaction temperature is lower than 90° C., there may be a problem of reduced synthesis yield due to an increase in unreacted products, and when it exceeds 130° C., the synthesis yield may decrease due to side reactions and, in addition, purification may become impossible. In addition, in step 3-2, the compound represented by [Chemical Formula 2] and diethyl phosphite may be mixed at a molar ratio of 1:1.3 to 1:5.3, preferably 1:2.3 to 1:4.3, and more preferably 1:3.0 to 1:3.6. If the molar ratio is less than 1:1.3, there may be a problem of reduced synthesis yield due to an increase in unreacted products, and if it exceeds 1:5.3, there may be a problem of an increase in side reaction products. In addition, in step 3-2, the compound represented by [Chemical Formula 2] and triphenylphosphine may be mixed at a molar ratio of 1:0.01 to 1:0.3, preferably 1:0.05 to 1:0.2, and more preferably 1:0.1 to 1:0.15. If the molar ratio is less than 1:0.01, there may be a problem of reduced synthesis yield due to an increase in unreacted products, and if it exceeds 1:0.3, there may be a problem of an increase in side reaction products. In addition, in step 3-2, the compound represented by [Chemical Formula 2] and cesium carbonate may be mixed at a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 4 of still another method for manufacturing the compound having an unshared electron pair for a perovskite solar cell of the present disclosure may include feeding hydrochloric acid (HCl) to the third reaction product prepared in step 3 and reacting them to produce the compound having an unshared electron pair for a perovskite solar cell represented by [Chemical Formula 1].

Specifically, step 4 of still another method for manufacturing the compound having an unshared electron pair for a perovskite solar cell of the present disclosure may include filtering the third reaction product prepared in step 3 to remove solid impurities and concentrating the organic phase of the third reaction product under reduced pressure to produce a concentrate.

Thereafter, hydrochloric acid (HCl) may be fed to the concentrate and stirred, and the resulting solid may be filtered and washed to produce the compound having an unshared electron pair for a perovskite solar cell represented by [Chemical Formula 1]. At this time, hydrochloric acid is preferably 4 to 8 N hydrochloric acid (HCl), but is not limited thereto. In addition, hydrochloric acid may be mixed such that the compound represented by [Chemical Formula 2] and hydrochloric acid are in a molar ratio of 1:5 to 1:10, preferably 1:6 to 1:9, and more preferably 1:7 to 1:8. If the molar ratio is less than 1:5, there may be a problem of reduced synthesis yield due to an increase in unreacted products, and if it exceeds 1:10, there may be a problem of an increase in side reaction products.

In addition, the stirring may be performed at a temperature of 60 to 100° C., preferably 70 to 90° C., for 1 to 8 hours, and preferably 3 to 5 hours.

Meanwhile, still another method for manufacturing the compound having an unshared electron pair for a perovskite solar cell of the present disclosure includes steps 1 to 3.

Step 1 of still another method for manufacturing the compound having an unshared electron pair for a perovskite solar cell of the present disclosure may include feeding the compound represented by [Chemical Formula 3] to phosphoryl chloride and reacting them to produce the compound represented by [Chemical Formula 2].

Specifically, step 1 of still another method for manufacturing the compound having an unshared electron pair for a perovskite solar cell of the present disclosure may include steps 1-1 to 1-3.

Step 1-1 of still another method for manufacturing the compound having an unshared electron pair for a perovskite solar cell of the present disclosure may include feeding the compound represented by [Chemical Formula 3] to phosphoryl chloride, and then refluxing with stirring at a temperature of 130 to 170° C., preferably 140 to 160° C., for 12 to 36 hours, preferably 18 to 30 hours, and more preferably 20 to 26 hours, to produce a first reaction product. At this time, when the reaction temperature is lower than 130° C., there may be a problem of reduced synthesis yield due to the presence of unreacted products, and when the temperature exceeds 170° C., the synthesis yield may decrease due to side reactions and, in addition, purification may become impossible.

Step 1-2 of still another method for manufacturing the compound having an unshared electron pair for a perovskite solar cell of the present disclosure may include neutralizing the first reaction product prepared in step 1-1 with a neutralizing agent, filtering a precipitate generated thereby to obtain a filtrate, and purifying the obtained filtrate to produce a purified product.

At this time, the neutralizing agent may include at least one selected from sodium hydroxide, sodium carbonate, potassium hydroxide, ammonium hydroxide, and sodium bicarbonate, and preferably may include sodium hydroxide. In addition, the purification may be performed through silica gel column chromatography.

Step 1-3 of still another method for manufacturing the compound having an unshared electron pair for a perovskite solar cell of the present disclosure may include concentrating the purified product prepared in step 1-2 under reduced pressure and then recrystallizing to produce the compound represented by [Chemical Formula 2].

At this time, the recrystallization may be performed using acetone, methanol, ethanol, isopropyl alcohol, or a mixture thereof, and may preferably be performed using acetone.

Step 2 of still another method for manufacturing the compound having an unshared electron pair for a perovskite solar cell of the present disclosure may include feeding ethyl 3-bromopropionate, potassium carbonate, and potassium iodide to the compound represented by [Chemical Formula 2] and reacting them to produce a second reaction product.

Specifically, step 2 of still another method for manufacturing the compound having an unshared electron pair for a perovskite solar cell of the present disclosure may include dissolving the compound represented by [Chemical Formula 2] in an organic solvent, feeding ethyl 3-bromopropionate, potassium carbonate, and potassium iodide, and stirring at a temperature of 90 to 130° C., preferably 100 to 120° C., for 5 to 15 hours, preferably 8 to 12 hours, and more preferably 9 to 11 hours, to produce a second reaction product.

At this time, when the stirring temperature is lower than 90° C., there may be a problem of reduced synthesis yield, and when it exceeds 130° C., there may be a problem of an increase in side reaction products. In addition, the organic solvent may include at least one selected from anhydrous dimethylformamide, dimethylacetamide, and dimethyl sulfoxide, and may preferably include anhydrous dimethylformamide.

In addition, ethyl 3-bromopropionate may be fed such that the compound represented by [Chemical Formula 2] and ethyl 3-bromopropionate are in a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

In addition, potassium carbonate may be fed such that the compound represented by [Chemical Formula 2] and potassium carbonate are in a molar ratio of 1:4 to 1:8, preferably 1:5 to 1:7, and more preferably 1:5.5 to 1:6.5. If the molar ratio is less than 1:4, there may be a problem of reduced synthesis yield due to an increase in unreacted products, and if it exceeds 1:8, there may be a problem of an increase in side reaction products.

In addition, potassium iodide may be fed such that the compound represented by [Chemical Formula 2] and potassium iodide are in a molar ratio of 1:0.05 to 1:0.5, preferably 1:0.1 to 1:0.4, and more preferably 1:0.15 to 1:0.25. If the molar ratio is less than 1:0.05, there may be a problem of reduced synthesis yield due to an increase in unreacted products, and if it exceeds 1:0.5, there may be a problem of an increase in side reaction products.

Step 3 of still another method for manufacturing the compound having an unshared electron pair for a perovskite solar cell of the present disclosure may include feeding sodium hydroxide (NaOH) to the second reaction product prepared in step 2 and reacting them to produce the compound having an unshared electron pair for a perovskite solar cell represented by [Chemical Formula 1].

Specifically, step 3 of still another method for manufacturing the compound having an unshared electron pair for a perovskite solar cell of the present disclosure may include steps 3-1 and 3-2.

Step 3-1 of still another method for manufacturing the compound having an unshared electron pair for a perovskite solar cell of the present disclosure may include extracting the second reaction product prepared in step 2 with an organic solvent, concentrating the organic phase of the extract under reduced pressure to obtain a concentrate, purifying the concentrate, and drying to obtain a dried product. At this time, the organic solvent may include at least one selected from dichloromethane, chloroform, and tetrahydrofuran, and may preferably include dichloromethane. In addition, the purification may be performed through silica gel chromatography.

Step 3-2 of still another method for manufacturing the compound having an unshared electron pair for a perovskite solar cell of the present disclosure may include dissolving the dried product obtained in step 3-1 in a mixed solvent, feeding sodium hydroxide (NaOH), and stirring and reacting at a temperature of 60 to 100° C., preferably 70 to 90° C., and more preferably 75 to 85° C., for 6 to 18 hours, preferably 10 to 14 hours, to produce a third reaction product, and filtering the third reaction product to produce the compound having an unshared electron pair for a perovskite solar cell represented by [Chemical Formula 1].

At this time, a mixed solvent including ethanol and water may be used, but is not limited thereto.

In addition, when the reaction temperature is lower than 60° C., there may be a problem of reduced synthesis yield due to an increase in unreacted products, and when it exceeds 100° C., the synthesis yield may decrease due to side reactions and, in addition, purification may become impossible.

In addition, the sodium hydroxide (NaOH) fed in step 3-2 may be a 1 to 3 N sodium hydroxide solution, but is not limited thereto.

Furthermore, the sodium hydroxide (NaOH) fed in step 3-2 may be such that the compound represented by [Chemical Formula 2] and sodium hydroxide (NaOH) are in a molar ratio of 1:3 to 1:15, preferably 1:4.5 to 1:12, and more preferably 1:6 to 1:9. If the molar ratio is less than 1:3, there may be a problem of reduced synthesis yield due to an increase in unreacted products, and if it exceeds 1:15, there may be a problem of an increase in side reaction products.

Furthermore, still another method for manufacturing the compound having an unshared electron pair for a perovskite solar cell of the present disclosure includes steps 1 to 4.

Step 1 of still another method for manufacturing the compound having an unshared electron pair for a perovskite solar cell of the present disclosure may include feeding the compound represented by [Chemical Formula 3] to phosphoryl chloride and reacting them to produce the compound represented by [Chemical Formula 2].

Specifically, step 1 of still another method for manufacturing the compound having an unshared electron pair for a perovskite solar cell of the present disclosure may include steps 1-1 to 1-3.

Step 1-1 of still another method for manufacturing the compound having an unshared electron pair for a perovskite solar cell of the present disclosure may include feeding the compound represented by [Chemical Formula 3] to phosphoryl chloride, and then refluxing with stirring at a temperature of 130 to 170° C., preferably 140 to 160° C., for 12 to 36 hours, preferably 18 to 30 hours, and more preferably 20 to 26 hours, to produce a first reaction product. At this time, when the reaction temperature is lower than 130° C., there may be a problem of reduced synthesis yield due to the presence of unreacted products, and when the temperature exceeds 170° C., the synthesis yield may decrease due to side reactions and, in addition, purification may become impossible.

Step 1-2 of still another method for manufacturing the compound having an unshared electron pair for a perovskite solar cell of the present disclosure may include neutralizing the first reaction product prepared in step 1-1 with a neutralizing agent, filtering a precipitate generated thereby to obtain a filtrate, and purifying the obtained filtrate to produce a purified product.

At this time, the neutralizing agent may include at least one selected from sodium hydroxide, sodium carbonate, potassium hydroxide, ammonium hydroxide, and sodium bicarbonate, and preferably may include sodium hydroxide. In addition, the purification may be performed through silica gel column chromatography.

Step 1-3 of still another method for manufacturing the compound having an unshared electron pair for a perovskite solar cell of the present disclosure may include concentrating the purified product prepared in step 1-2 under reduced pressure and then recrystallizing to produce the compound represented by [Chemical Formula 2].

At this time, the recrystallization may be performed using acetone, methanol, ethanol, isopropyl alcohol, or a mixture thereof, and may preferably be performed using acetone.

Step 2 of still another method for manufacturing the compound having an unshared electron pair for a perovskite solar cell of the present disclosure may include feeding 1,3-dibromoalkane, potassium carbonate, and potassium iodide to the compound represented by [Chemical Formula 2] and reacting them to produce a second reaction product.

Specifically, step 2 of still another method for manufacturing the compound having an unshared electron pair for a perovskite solar cell of the present disclosure may include dissolving the compound represented by [Chemical Formula 2] in an organic solvent, feeding 1,3-dibromoalkane, potassium carbonate, and potassium iodide, and stirring at a temperature of 90 to 130° C., preferably 100 to 120° C., for 5 to 15 hours, preferably 8 to 12 hours, and more preferably 9 to 11 hours, to produce a second reaction product.

At this time, when the stirring temperature is lower than 90° C., there may be a problem of reduced synthesis yield, and when it exceeds 130° C., there may be a problem of an increase in side reaction products. In addition, the organic solvent may include at least one selected from anhydrous dimethylformamide, dimethylacetamide, and dimethyl sulfoxide, and may preferably include anhydrous dimethylformamide.

In addition, 1,3-dibromoalkane may be fed such that the compound represented by [Chemical Formula 2] and 1,3-dibromoalkane are in a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

In addition, potassium carbonate may be fed such that the compound represented by [Chemical Formula 2] and potassium carbonate are in a molar ratio of 1:4 to 1:8, preferably 1:5 to 1:7, and more preferably 1:5.5 to 1:6.5. If the molar ratio is less than 1:4, there may be a problem of reduced synthesis yield due to an increase in unreacted products, and if it exceeds 1:8, there may be a problem of an increase in side reaction products.

In addition, potassium iodide may be fed such that the compound represented by [Chemical Formula 2] and potassium iodide are in a molar ratio of 1:0.05 to 1:0.5, preferably 1:0.1 to 1:0.4, and more preferably 1:0.15 to 1:0.25. If the molar ratio is less than 1:0.05, there may be a problem of reduced synthesis yield due to an increase in unreacted products, and if it exceeds 1:0.5, there may be a problem of an increase in side reaction products.

Step 3 of still another method for manufacturing the compound having an unshared electron pair for a perovskite solar cell of the present disclosure may include feeding sodium sulfite to the second reaction product and reacting them to produce a third reaction product.

Specifically, step 3 of still another method for manufacturing the compound having an unshared electron pair for a perovskite solar cell of the present disclosure may include steps 3-1 and 3-2.

Step 3-1 of still another method for manufacturing the compound having an unshared electron pair for a perovskite solar cell of the present disclosure may include extracting the second reaction product prepared in step 2 with an organic solvent, concentrating the organic phase of the extract under reduced pressure to obtain a concentrate, purifying the concentrate, and drying to obtain a dried product. At this time, the organic solvent may include at least one selected from dichloromethane, chloroform, and tetrahydrofuran, and may preferably include dichloromethane. In addition, the purification may be performed through silica gel chromatography.

Step 3-2 of still another method for manufacturing the compound having an unshared electron pair for a perovskite solar cell of the present disclosure may include dissolving the dried product obtained in step 3-1 in a mixed solvent, feeding sodium sulfite, and stirring and reacting at a temperature of 60 to 100° C., preferably 70 to 90° C., and more preferably 75 to 85° C., for 3 to 9 hours, preferably 5 to 7 hours, to produce a third reaction product.

At this time, a mixed solvent including ethanol and water may be used, but is not limited thereto.

In addition, when the reaction temperature is lower than 60° C., there may be a problem of reduced synthesis yield due to an increase in unreacted products, and when it exceeds 100° C., the synthesis yield may decrease due to side reactions and, in addition, purification may become impossible.

In addition, sodium sulfite fed in step 3-2 may be such that the compound represented by [Chemical Formula 2] and sodium sulfite are in a molar ratio of 1:1 to 1:5, preferably 1:2 to 1:4, and more preferably 1:2.5 to 1:3.5. If the molar ratio is less than 1:1, there may be a problem of reduced synthesis yield due to an increase in unreacted products, and if it exceeds 1:5, there may be a problem of an increase in side reaction products.

Step 4 of still another method for manufacturing the compound having an unshared electron pair for a perovskite solar cell of the present disclosure may include feeding hydrochloric acid (HCl) to the third reaction product prepared in step 3 and reacting them to produce the compound having an unshared electron pair for a perovskite solar cell represented by [Chemical Formula 1].

Specifically, step 4 of still another method for manufacturing the compound having an unshared electron pair for a perovskite solar cell of the present disclosure may include cooling the third reaction product prepared in step 3 to a temperature of 20 to 30° C., feeding hydrochloric acid (HCl), and stirring to produce the compound having an unshared electron pair for a perovskite solar cell represented by [Chemical Formula 1].

At this time, the hydrochloric acid (HCl) fed in step 4 may be a 5 to 7 N hydrochloric acid solution, but is not limited thereto.

In addition, hydrochloric acid (HCl) fed in step 4 may be such that the compound represented by [Chemical Formula 2] and hydrochloric acid (HCl) are in a molar ratio of 1:5 to 1:10, preferably 1:6 to 1:9, and more preferably 1:7 to 1:8. If the molar ratio is less than 1:5, there may be a problem of reduced synthesis yield due to an increase in unreacted products, and if it exceeds 1:10, there may be a problem of an increase in side reaction products.

Furthermore, the perovskite solar cell of the present disclosure may include a laminate in which a hole transport layer, an organic interfacial layer, and a perovskite light-absorbing layer are sequentially stacked.

The hole transport layer (HTL) is a layer that transports holes formed in the perovskite light-absorbing layer, which will be described later, while blocking the movement of electrons, and may include inorganic and/or organic hole transport materials.

At this time, the inorganic hole transport material may include at least one selected from nickel oxide (NiOx), CuSCN, CuCrO₂, CuI, MoO, and V₂O₅.

In addition, the organic hole transport material may include at least one selected from carbazole derivatives, polyarylalkane derivatives, phenylenediamine derivatives, arylamine derivatives, amino-substituted chalcone derivatives, styrylanthracene derivatives, fluorene derivatives, hydrazone derivatives, stilbene derivatives, silazane derivatives, aromatic tertiary amine compounds, styrylamine compounds, aromatic dimethylidene-based compounds, porphyrin-based compounds, phthalocyanine-based compounds, polythiophene derivatives, polypyrrole derivatives, polyparaphenylene vinylene derivatives, pentacene, coumarin 6 (3-(2-benzothiazolyl)-7-(diethylamino) coumarin), ZnPC (zinc phthalocyanine), CuPC (copper phthalocyanine), TiOPC (titanium oxide phthalocyanine), Spiro-MeOTAD ([2,2′,7,7′-tetrakis(N,N-p-dimethoxyphenylamino)-9,9′-spirobifluorene]), F16CuPC (copper(II) 1,2,3,4,8,9,10,11,15,16,17,18,22,23,24,25-hexadecafluoro-29H,31H-phthalocyanine), SubPc (boron subphthalocyanine chloride), N3 (cis-di(thiocyanato)-bis(2,2′-bipyridyl-4,4′-dicarboxylic acid)-ruthenium (II)), P3HT (poly[3-hexylthiophene]), MDMO-PPV (poly[2-methoxy-5-(3′,7′-dimethyloctyloxyl)-1,4-phenylene vinylene]), MEH-PPV (poly[2-methoxy-5-(2″-ethylhexyloxy)-p-phenylene vinylene]), P3OT (poly(3-octyl thiophene)), POT (poly(octyl thiophene)), P3DT (poly(3-decyl thiophene)), P3DDT (poly(3-dodecyl thiophene)), PPV (poly(p-phenylene vinylene)), TFB (poly(9,9′-dioctylfluorene-co-N-(4-butylphenyl)diphenyl amine)), polyaniline, Spiro-MeOTAD ([2,2′,7,7′-tetrakis(N,N-di-p-methoxyphenyl amine)-9,9′-spirobifluorene]), PCPDTBT (poly[2,1,3-benzothiadiazole-4,7-diyl][4,4-bis(2-ethylhexyl-4H-cyclopenta[2,1-b:3,4-b′]dithiophene-2,6-diyl])), Si-PCPDTBT (poly[(4,4′-bis(2-ethylhexyl)dithieno[3,2-b:2′,3′-d]silole)-2,6-diyl-alt-(2,1,3-benzothiadiazole)-4,7-diyl]), PBDTTPD (poly(4,8-diethylhexyloxy . . . )), PFDTBT (poly[2,7-(9-(2-ethylhexyl)-9-hexyl-fluorene)-alt-5,5-(4′,7′-di-2-thienyl-2′,1′,3′-benzothiadiazole)]), PFO-DBT (poly[2,7-9,9′-(dioctylfluorene)-alt-5,5-(4′,7′-di-2-thienyl-2′,1′,3′-benzothiadiazole)]), PSiFDTBT (poly[(2,7-dioctylsilafluorene)-2,7-diyl-alt-(4,7-bis(2-thienyl)-2,1,3-benzothiadiazole)-5,5′-diyl]), PCDTBT (poly[[9-(1-octylnonyl)-9H-carbazole-2,7-diyl]-2,5-thiophenediyl-2,1,3-benzothiadiazole-4,7-diyl-2,5-thiophenediyl]), PFB (poly(9,9′-dioctylfluorene-co-bis(N,N′-(4-butylphenyl))bis(N,N′-phenyl-1,4-phenylene)diamine)), F8BT (poly(9,9′-dioctylfluorene-co-benzothiadiazole)), PEDOT (poly(3,4-ethylenedioxythiophene)), PEDOT:PSS (poly(3,4-ethylenedioxythiophene) poly(styrenesulfonate)), PTAA (poly(triarylamine)), 2-PACz, MeO-2PACz, Br-2PACz, Me-4PACz, MeO-4PACz, and 6-PACz.

In addition, examples of methods for forming the hole transport layer include coating methods and vacuum deposition methods. The coating methods may include gravure coating, bar coating, printing, spraying, spin coating, dipping, and die coating.

In addition, the thickness of the hole transport layer is not particularly limited, but may be preferably 5 nm to 40 nm, and more preferably 10 nm to 30 nm.

The organic interfacial layer may include the compound having an unshared electron pair for a perovskite solar cell represented by [Chemical Formula 1], as described above.

In addition, examples of methods for forming the organic interfacial layer include coating methods and vacuum deposition methods. The coating methods may include gravure coating, bar coating, printing, spraying, spin coating, dipping, and die coating.

In addition, the thickness of the organic interfacial layer is not particularly limited, but may preferably be 0.5 nm to 10 nm, and more preferably 1 nm to 5 nm.

The perovskite light-absorbing layer may include a perovskite material represented by [Chemical Formula 1] below.

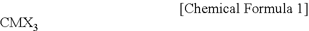

In [Chemical Formula 1], C is a monovalent cation and may include an amine, ammonium, a group 1 metal, a group 2 metal, and/or another cation or cation-like compound. Preferably, C may be formamidinium (FA), methylammonium (MA), FAMA, CsFAMA, CsFA, or N(R)⁴⁺ (where R may be the same or different, and R is a C1-C5 linear alkyl group, a C3-C5 branched alkyl group, a phenyl group, an alkylphenyl group, an alkoxyphenyl group, or an alkyl halide).

In addition, M in [Chemical Formula 1] is a divalent cation and may include one or two selected from Fe, Co, Ni, Cu, Sn, Pb, Bi, Ge, Ti, Eu, and Zr.

In addition, X in [Chemical Formula 1] is a monovalent anion and may include one or more halogen elements selected from F, Cl, Br, and I and/or group 16 anions. As a preferred example, X may be IₓBr₃₋ₓ (0≤x≤3).

In addition, as a preferred embodiment of [Chemical Formula 1], examples may include FAPbIₓBr₃₋ₓ (0≤x≤3), MAPbIₓBr₃₋ₓ (0≤x≤3), CSFAPbIₓBr₃₋ₓ (0≤x≤3), CSMAFAPbIₓBr₃₋ₓ (0≤x≤3), CH₃NH₃PbX₃ (X═Cl, Br, I, BrI₂, or Br₂I), CH₃NH₃SnX₃ (X═Cl, Br or I), CH(═NH)NH₃PbX₃ (X═Cl, Br, I, BrI₂, or Br₂I) or CH(═NH)NH₃SnX₃ (X═Cl, Br or I).

Meanwhile, the perovskite light-absorbing layer may be a single layer composed of the same perovskite material, or a multilayer structure in which layers composed of different perovskite materials are stacked. In addition, within the light-absorbing layer formed of one type of perovskite material, a different type of perovskite material may be included in the form of pillars, plates, needles, wires, or rods.

In addition, the perovskite light-absorbing layer may be formed by deposition processes or solution processes. The deposition processes may include any commonly used deposition processes in the art, such as thermal evaporation, vacuum deposition, atomic layer deposition (ALD), chemical vapor deposition (CVD), and physical vapor deposition (PVD). The solution processes may include any commonly used solution processes in the art, such as spin coating, slot-die coating, blade coating, printing coating, gravure coating, and spray coating.

In addition, the thickness of the perovskite light-absorbing layer is not particularly limited, but may be preferably 50 nm to 800 nm, and more preferably 300 nm to 600 nm.

In addition, the perovskite solar cell of the present disclosure may further include an electron transport layer formed on one surface of the perovskite light-absorbing layer.

The electron transport layer (ETL) is a layer that transports electrons generated in the perovskite light-absorbing layer while blocking the movement of holes.

The electron transport layer may include one or more selected from tin oxide (SnOx), nickel oxide (NiOx), tin dioxide (SnO₂), titanium dioxide (TiO₂), zinc oxide (ZnO), barium stannate (BaSnO₃), niobium hydroxide (NbOH), and niobium pentoxide (Nb₂O₅).

In addition, the electron transport layer may include an inorganic material and/or an organic material.

In this case, the inorganic material may include one or more selected from nickel oxide (NiOx), CuSCN, CuCrO₂, CuI, CuOx, CuS, CuPc, CIS, CuGaO₂, PbS, MoOx, AlOx (aluminum oxide), CuAlOx, aluminum oxide nanoparticles, silica nanoparticles, nickel oxide nanoparticles, hafnium nanoparticles, and V₂O₅.

In addition, the organic material may include at least one selected from carbazole derivatives, polyarylalkane derivatives, phenylenediamine derivatives, arylamine derivatives, amino-substituted chalcone derivatives, styrylanthracene derivatives, fluorene derivatives, hydrazone derivatives, stilbene derivatives, silazane derivatives, aromatic tertiary amine compounds, styrylamine compounds, aromatic dimethylidene-based compounds, porphyrin-based compounds, phthalocyanine-based compounds, polythiophene derivatives, polypyrrole derivatives, polyparaphenylene vinylene derivatives, pentacene, coumarin 6 (3-(2-benzothiazolyl)-7-(diethylamino) coumarin), ZnPC (zinc phthalocyanine), CuPC (copper phthalocyanine), TiOPC (titanium oxide phthalocyanine), Spiro-MeOTAD (2,2′,7,7′-tetrakis(N,N-p-dimethoxyphenylamino)-9,9′-spirobifluorene), F16CuPC (copper (II) 1,2,3,4,8,9,10,11,15,16,17,18,22,23,24,25-hexadecafluoro-29H,31H-phthalocyanine), SubPc (boron subphthalocyanine chloride), N3 (cis-di(thiocyanato)-bis(2,2′-bipyridyl-4,4′-dicarboxylic acid)-ruthenium (II)), P3HT (poly[3-hexylthiophene]), MDMO-PPV (poly[2-methoxy-5-(3′,7′-dimethyloctyloxy)-1,4-phenylene vinylene]), MEH-PPV (poly[2-methoxy-5-(2″-ethylhexyloxy)-p-phenylene vinylene]), P3OT (poly(3-octyl thiophene)), POT (poly(octyl thiophene)), P3DT (poly(3-decyl thiophene)), P3DDT (poly(3-dodecyl thiophene)), PPV (poly(p-phenylene vinylene)), TFB (poly(9,9′-dioctylfluorene-co-N-(4-butylphenyl)diphenyl amine)), polyaniline, Spiro-MeOTAD (2,2′,7,7′-tetrakis(N,N-di-p-methoxyphenyl amine)-9,9′-spirobifluorene), PCPDTBT (poly[2,1,3-benzothiadiazole-4,7-diyl][4,4-bis(2-ethylhexyl-4H-cyclopenta[2,1-b:3,4-b′]dithiophene-2,6-diyl]]), Si-PCPDTBT (poly[(4,4′-bis(2-ethylhexyl)dithieno[3,2-b:2′,3′-d]silole)-2,6-diyl-alt-(2,1,3-benzothiadiazole)-4,7-diyl]), PBDTTPD (poly(4,8-diethylhexyloxy . . . )), PFDTBT (poly[2,7-(9-(2-ethylhexyl)-9-hexyl-fluorene)-alt-5,5-(4′,7′-di-2-thienyl-2′,1′,3′-benzothiadiazole)]), PFO-DBT (poly[2,7-9,9′-(dioctylfluorene)-alt-5,5-(4′,7′-di-2-thienyl-2′,1′,3′-benzothiadiazole)]), PSiFDTBT (poly[(2,7-dioctylsilafluorene)-2,7-diyl-alt-(4,7-bis(2-thienyl)-2,1,3-benzothiadiazole)-5,5′-diyl]), PCDTBT (poly[[9-(1-octylnonyl)-9H-carbazole-2,7-diyl]-2,5-thiophenediyl-2,1,3-benzothiadiazole-4,7-diyl-2,5-thiophenediyl]), PFB (poly(9,9′-dioctylfluorene-co-bis(N,N′-(4-butylphenyl))bis(N,N′-phenyl-1,4-phenylene)diamine)), F8BT (poly(9,9′-dioctylfluorene-co-benzothiadiazole)), PEDOT (poly(3,4-ethylenedioxythiophene)), PEDOT:PSS (poly(3,4-ethylenedioxythiophene) poly(styrenesulfonate)), PTAA (poly(triarylamine)), 2-PACz, MeO-2PACz, Br-2PACz, Me-4PACz, MeO-4PACz, and 6-PACz.

In addition, the electron transport layer may include a fullerene-based organic material. In this case, the fullerene-based organic material may include at least one selected from C60, C70, PC60BM, and PC70BM.

In addition, the electron transport layer may be formed by deposition processes or solution processes. The deposition processes may include any commonly used deposition processes in the art, such as thermal evaporation, vacuum deposition, atomic layer deposition (ALD), chemical vapor deposition (CVD), and physical vapor deposition (PVD). The solution processes may include any commonly used solution processes in the art, such as spin coating, slot-die coating, blade coating, printing coating, gravure coating, and spray coating.

In addition, the electron transport layer is not particularly limited in thickness, but may preferably have an average thickness of 3 to 300 nm, and more preferably 5 to 200 nm.

In addition, the perovskite solar cell of the present disclosure may further include a transparent electrode formed on one surface of the electron transport layer.

The transparent electrode may be formed through a deposition process. In this case, the deposition may be performed by a general deposition process used in the art, and preferably by a sputtering method.

In addition, the transparent electrode may be a transparent thin film deposited with ITO (indium tin oxide), FTO (fluorine-doped tin oxide), ATO ((Sb₂O₃ (antimony oxide)-doped tin oxide), GTO (gallium-doped tin oxide), ZTO (tin-doped zinc oxide), ZTO:Ga (gallium-doped ZTO), IGZO (indium gallium zinc oxide), IZO (indium-doped zinc oxide), or AZO (aluminum-doped zinc oxide).

In addition, the transparent electrode is not particularly limited in thickness, but may preferably have a thickness of 50 to 200 nm, and more preferably 60 to 140 nm.

In addition, the perovskite solar cell of the present disclosure may further include a metal electrode formed on one surface of the transparent electrode.

The metal electrode may be formed on one surface of the transparent electrode by patterning a metal material. Specifically, the patterning process generally includes deposition, lithography, and etching, in which a thin film of a metal material is formed on one surface of a substrate, a pattern is imprinted by lithography, and unnecessary portions are removed, thereby forming the metal electrode on the transparent electrode. In addition, the patterning process may be performed by screen printing using a metal paste containing a metal material. More specifically, the metal electrode may include a plurality of bus bar electrodes arranged in a horizontal direction and a plurality of finger electrodes arranged in a vertical direction of the bus bar electrodes.

In addition, the metal material may include at least one selected from Pt, Au, Ni, Cu, Ag, In, Ru, Pd, Rh, Ir, Os, C, and conductive polymers.

Further, the thickness of the metal electrode is not particularly limited, but may preferably be 50 nm to 15 μm.

Meanwhile, a tandem perovskite solar cell of the present disclosure may include a laminate in which a solar cell, a recombination layer, a hole transport layer, an organic interfacial layer, and a perovskite light-absorbing layer are sequentially stacked. Here, each of the hole transport layer, the organic interfacial layer, and the perovskite light-absorbing layer is as described above.

In addition, the tandem perovskite solar cell of the present disclosure may further include an electron transport layer formed on one surface of the perovskite light-absorbing layer, and the electron transport layer is as described above.

In addition, the tandem perovskite solar cell of the present disclosure may further include a transparent electrode formed on one surface of the electron transport layer, and the transparent electrode is as described above.

In addition, the tandem perovskite solar cell of the present disclosure may further include a metal electrode formed on one surface of the transparent electrode, and the metal electrode is as described above.

The solar cell may be a polycrystalline silicon solar cell, a crystalline silicon solar cell, a perovskite solar cell, a gallium arsenide (GaAs) solar cell, a cadmium telluride (CdTe) solar cell, a CIGS (CuInGaSe) solar cell, a CZTS (Cu₂ZnSnS₄) solar cell, an organic solar cell, a dye-sensitized solar cell, or a group III-V compound solar cell.

In addition, the thickness of the solar cell is not particularly limited, but may preferably be 140 to 250 μm, and more preferably 160 to 200 μm.

In addition, when a silicon solar cell doped with an n-type or p-type impurity is used as the solar cell, the silicon solar cell doped with the n-type or p-type impurity may be subjected to hydrofluoric acid treatment to remove an SiOx oxide film, and then residual hydrofluoric acid may be removed using ultrapure water before being used.

The recombination layer is a layer that induces the recombination of electrons and holes generated in the solar cell and the perovskite light-absorbing layer, and may be a transparent thin film deposited with ITO (indium tin oxide), FTO (fluorine-doped tin oxide), ATO (Sb₂O₃ (antimony oxide)-doped tin oxide), GTO (gallium-doped tin oxide), ZTO (tin-doped zinc oxide), ZTO:Ga (gallium-doped ZTO), IGZO (indium gallium zinc oxide), IZO (indium-doped zinc oxide), or AZO (aluminum-doped zinc oxide).

In addition, in one example of forming the recombination layer, the recombination layer may be formed on one surface of the solar cell through a sputtering process.

In addition, the thickness of the recombination layer is not particularly limited, but may preferably be 5 nm to 50 nm, and more preferably 15 nm to 25 nm.

Hereinafter, the present disclosure will be described in more detail through the following Examples, but the following Examples are not intended to limit the scope of the present disclosure, which should be construed merely as being provided for better understanding of the present disclosure.

### Example 1-1: Preparation of a Compound Having an Unshared Electron Pair for a Perovskite Solar Cell

(1) Under an argon atmosphere, 223.65 mmol of a compound represented by [Chemical Formula 3-1] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-1] below.
(4) The prepared compound represented by [Chemical Formula 2-1] below was dissolved in 50 ml of anhydrous dimethyl formamide, sodium hydride was added, and the mixture was stirred at 25° C. for 10 minutes to prepare a mixture. At this time, the compound represented by [Chemical Formula 2-1] below and sodium hydride were fed in a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-1] below and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous tetrahydrofuran at 0° C., and 50 ml of n-butyllithium was dropwise added. Then, trimethyl borate was dropwise added, and the mixture was stirred and reacted at 25° C. for 12 hours to produce a third reaction product. At this time, trimethyl borate was added so that the compound represented by [Chemical Formula 2-1] below and trimethyl borate were in a molar ratio of 1:3.0.
(8) 100 ml of 1 N hydrochloric acid (HCl) solution (containing 100 mmol of HCl) was dropwise added to the prepared third reaction product, the mixture was stirred to produce a fourth reaction product, and the organic phase of the prepared fourth reaction product was then separated, concentrated under reduced pressure, and purified through silica gel chromatography to manufacture a compound having an unshared electron pair for a perovskite solar cell represented by [Chemical Formula 1-1] below. At this time, hydrochloric acid (HCl) was added so that the compound represented by [Chemical Formula 2-1] below and hydrochloric acid were in a molar ratio of 1:7.

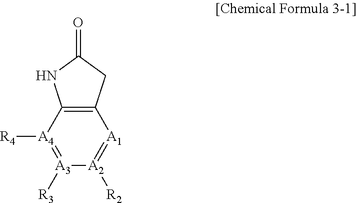

In [Chemical Formula 3-1], A₂, A₃, and A₄ are each carbon (C), A₁ is nitrogen (N), and R₂, R₃, and R₄ are each —H.

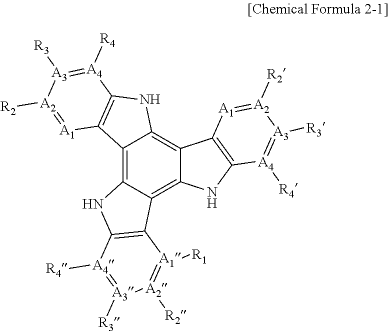

In [Chemical Formula 2-1], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), and R₂, R₃, R₄, R_{2′}, R_{3′}, R_{4′}, R_{2″}, R_{3″}, and R_{4″} are each —H.

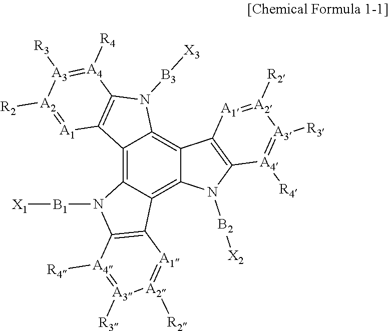

In [Chemical Formula 1-1], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), R₂, R₃, R₄, R_{2′}, R_{3′}, R_{4′}, R_{2″}, R_{3″}, and R_{4″} are each —H, B₁, B₂, and B₃ are each

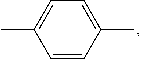

and X₁, X₂, and X₃ are each a boronic acid group (—B(OH)₂).

### Example 1-2: Preparation of a Compound Having an Unshared Electron Pair for a Perovskite Solar Cell

(1) Under an argon atmosphere, 223.65 mmol of a compound represented by [Chemical Formula 3-2] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-2] below.
(4) The prepared compound represented by [Chemical Formula 2-2] below was dissolved in 50 ml of anhydrous dimethyl formamide, sodium hydride was added, and the mixture was stirred at 25° C. for 10 minutes to prepare a mixture. At this time, the compound represented by [Chemical Formula 2-2] below and sodium hydride were fed in a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-2] below and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous tetrahydrofuran at 0° C., and 50 ml of n-butyllithium was dropwise added. Then, trimethyl borate was dropwise added, and the mixture was stirred and reacted at 25° C. for 12 hours to produce a third reaction product. At this time, trimethyl borate was added so that the compound represented by [Chemical Formula 2-2] below and trimethyl borate were in a molar ratio of 1:3.0.
(8) 100 ml of 1 N hydrochloric acid (HCl) solution (containing 100 mmol of HCl) was dropwise added to the prepared third reaction product, the mixture was stirred to produce a fourth reaction product, and the organic phase of the prepared fourth reaction product was then separated, concentrated under reduced pressure, and purified through silica gel chromatography to manufacture a compound having an unshared electron pair for a perovskite solar cell represented by [Chemical Formula 1-2] below. At this time, hydrochloric acid (HCl) was added so that the compound represented by [Chemical Formula 2-2] below and hydrochloric acid were in a molar ratio of 1:7.

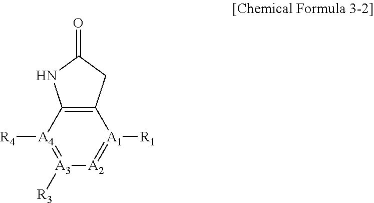

In [Chemical Formula 3-2], A₁, A₃, and A₄ are each carbon (C), A₂ is nitrogen (N), and R₁, R₃, and R₄ are each —H.

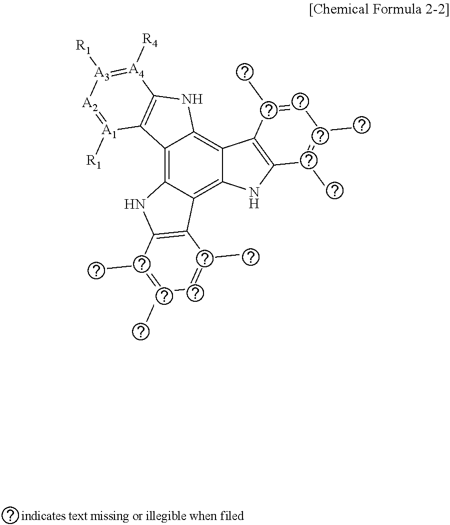

In [Chemical Formula 2-2], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), and R₁, R₃, R₄, R_{1′}, R_{3′}, R_{4′}, R_{1″}, R_{3″}, and R_{4″} are each —H.

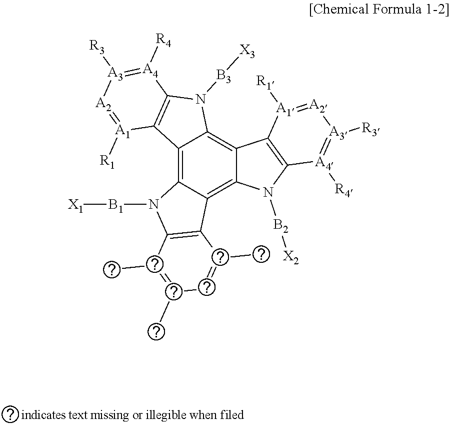

In [Chemical Formula 1-2], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), R₁, R₃, R₄, R_{1′}, R_{3′}, R_{4′}, R_{1″}, R_{3″}, and R_{4″} are each —H, B₁, B₂, and B₃ are each

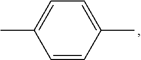

and X₁, X₂, and X₃ are each a boronic acid group (—B(OH)₂).

### Example 1-3: Preparation of a Compound Having an Unshared Electron Pair for a Perovskite Solar Cell

(1) Under an argon atmosphere, 223.65 mmol of a compound represented by [Chemical Formula 3-3] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was silica gel column chromatography (eluent: dichloromethane) to prepare a purified product.
(3) The prepared purified product was concentrated under reduced pressure and recrystallized using acetone to prepare a compound represented by [Chemical Formula 2-3] below.
(4) The prepared compound represented by [Chemical Formula 2-3] below was dissolved in 50 ml of anhydrous dimethyl formamide, sodium hydride was added, and the mixture was stirred at 25° C. for 10 minutes to prepare a mixture. At this time, the compound represented by [Chemical Formula 2-3] below and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-3] below and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous tetrahydrofuran at 0° C., and 50 ml of n-butyllithium was dropwise added. Then, trimethyl borate was dropwise added, and the mixture was stirred and reacted at 25° C. for 12 hours to produce a third reaction product. At this time, trimethyl borate was added so that the compound represented by [Chemical Formula 2-3] below and trimethyl borate were in a molar ratio of 1:3.0.
(8) 100 ml of 1 N hydrochloric acid (HCl) solution (containing 100 mmol of HCl) was dropwise added to the prepared third reaction product, the mixture was stirred to produce a fourth reaction product, and the organic phase of the prepared fourth reaction product was then separated, concentrated under reduced pressure, and purified through silica gel chromatography to manufacture a compound having an unshared electron pair for a perovskite solar cell represented by [Chemical Formula 1-3] below. At this time, hydrochloric acid (HCl) was added so that the compound represented by [Chemical Formula 2-3] below and hydrochloric acid were in a molar ratio of 1:7.

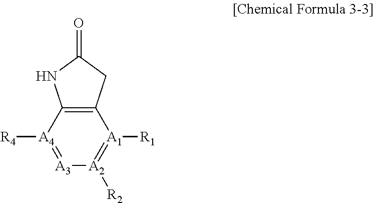

In [Chemical Formula 3-3], A₁, A₂, and A₄ are each carbon (C), A₃ is nitrogen (N), and R₁, R₂, and R₄ are each —H.

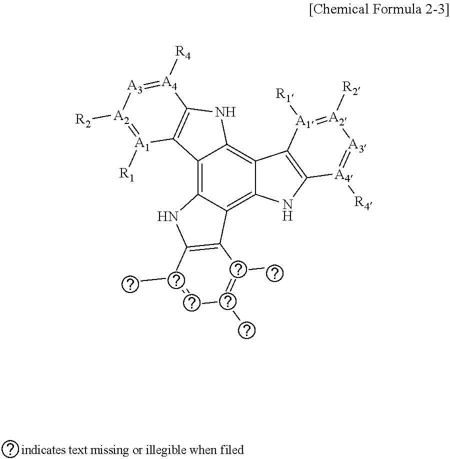

In [Chemical Formula 2-3], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), and R₁, R₂, R₄, R_{1′}, R_{2′}, R_{4′}, R_{1″}, R_{2″}, and R_{4″} are each —H.

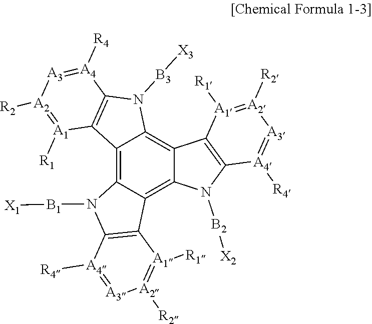

In [Chemical Formula 1-3], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), R₁, R₂, R₄, R_{1′}, R_{2′}, R_{4′}, R_{1″}, R_{2″}, and R_{4″} are each —H, B₁, B₂, and B₃ are each

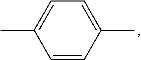

and X₁, X₂, and X₃ are each a boronic acid group (—B(OH)₂).

### Example 1-4: Preparation of a Compound Having an Unshared Electron Pair for a Perovskite Solar Cell

(1) Under an argon atmosphere, 223.65 mmol of a compound represented by [Chemical Formula 3-4] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-4] below.
(4) The prepared compound represented by [Chemical Formula 2-4] below was dissolved in 50 ml of anhydrous dimethyl formamide, sodium hydride was added, and the mixture was stirred at 25° C. for 10 minutes to prepare a mixture. At this time, the compound represented by [Chemical Formula 2-4] below and sodium hydride were fed in a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-4] below and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous tetrahydrofuran at 0° C., and 50 ml of n-butyllithium was dropwise added. Then, trimethyl borate was dropwise added, and the mixture was stirred and reacted at 25° C. for 12 hours to produce a third reaction product. At this time, trimethyl borate was added so that the compound represented by [Chemical Formula 2-4] below and trimethyl borate were in a molar ratio of 1:3.0.
(8) 100 ml of 1 N hydrochloric acid (HCl) solution (containing 100 mmol of HCl) was dropwise added to the prepared third reaction product, the mixture was stirred to produce a fourth reaction product, and the organic phase of the prepared fourth reaction product was then separated, concentrated under reduced pressure, and purified through silica gel chromatography to manufacture a compound having an unshared electron pair for a perovskite solar cell represented by [Chemical Formula 1-4] below. At this time, hydrochloric acid (HCl) was added so that the compound represented by [Chemical Formula 2-4] below and hydrochloric acid were in a molar ratio of 1:7.

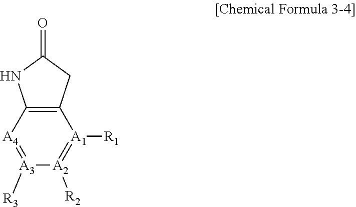

In [Chemical Formula 3-4], A₁, A₂, and A₃ are each carbon (C), A₄ is nitrogen (N), and R₁, R₂, and R₃ are each —H.

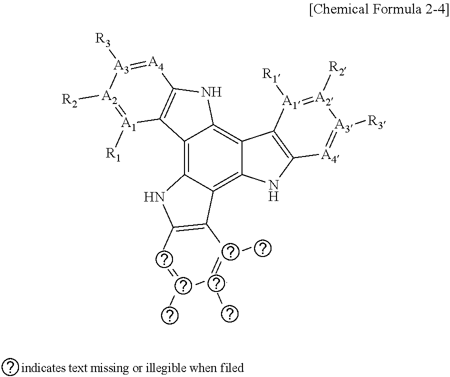

In [Chemical Formula 2-4], A₁, A₂, A₃, A_{1′}, A_{2′}, A_{3′}, A_{1″}, A_{2″}, and A_{3″} are each carbon (C), A₄, A_{4′}, and A_{4″} are each nitrogen (N), and R₁, R₂, R₃, R_{1′}, R_{2′}, R_{3′}, R_{1″}, R_{2″}, and R_{3″} are each —H.

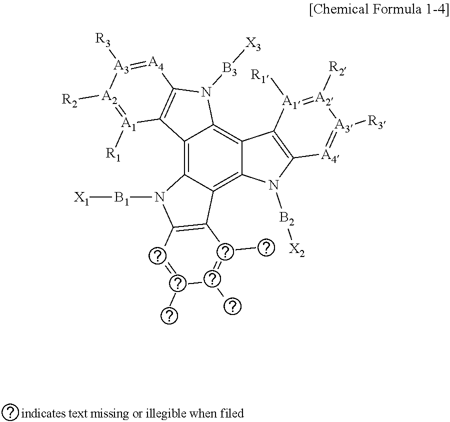

In [Chemical Formula 1-4], A₁, A₂, A₃, A_{1′}, A_{2′}, A_{3′}, A_{1″}, A_{2″}, and A_{3″} are each carbon (C), A₄, A_{4′}, and A_{4″} are each nitrogen (N), R₁, R₂, R₃, R_{1′}, R_{2′}, R_{3′}, R_{1″}, R_{2″}, and R_{3″} are each —H, B₁, B₂, and B₃ are each

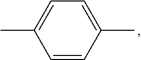

and X₁, X₂, and X₃ are each a boronic acid group (—B(OH)₂).

### Example 1-5: Preparation of a Compound Having an Unshared Electron Pair for a Perovskite Solar Cell

(1) Under an argon atmosphere, 223.65 mmol of the compound represented by [Chemical Formula 3-2] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-2].
(4) The compound represented by [Chemical Formula 2-2] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-2] and sodium hydride were added at a molar ratio of 1:3.1.
(5) 1,3-dibromobenzene was added to the prepared mixture, and the mixture was stirred at 100° C. for 10 hours to produce a second reaction product. At this time, 1,3-dibromobenzene was added so that the compound represented by [Chemical Formula 2-2] and 1,3-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous tetrahydrofuran at 0° C., and 50 ml of n-butyllithium was dropwise added. Then, trimethyl borate was dropwise added, and the mixture was stirred and reacted at 25° C. for 12 hours to produce a third reaction product. At this time, trimethyl borate was added so that the compound represented by [Chemical Formula 2-2] and trimethyl borate were in a molar ratio of 1:3.0.
(8) 100 ml of 1 N hydrochloric acid (HCl) solution (containing 100 mmol of HCl) was dropwise added to the prepared third reaction product, the mixture was stirred to produce a fourth reaction product, and the organic phase of the prepared fourth reaction product was then separated, concentrated under reduced pressure, and purified through silica gel chromatography to manufacture a compound having an unshared electron pair for a perovskite solar cell represented by [Chemical Formula 1-5] below. At this time, hydrochloric acid (HCl) was added so that the compound represented by [Chemical Formula 2-2] and hydrochloric acid were in a molar ratio of 1:7.

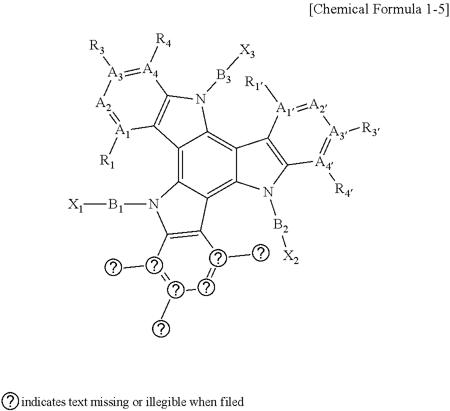

In [Chemical Formula 1-5], A₁, A₃, A₄, A₁, A_{3′}, A_{4′}, A_{1″}, A_{3″}, and A_{4″} are each carbon (C), A₂, A_{2′}, and A_{2″} are each nitrogen (N), R₁, R₃, R₄, R_{1′}, R_{3′}, R_{4′}, R_{1″}, R_{3″}, and R_{4″} are each —H, B₁, B₂, and B₃ are each

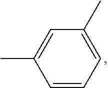

and X₁, X₂, and X₃ are each a boronic acid group (—B(OH)₂).

### Example 1-6: Preparation of a Compound Having an Unshared Electron Pair for a Perovskite Solar Cell

(1) Under an argon atmosphere, 223.65 mmol of the compound represented by [Chemical Formula 3-1] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-1].
(4) The compound represented by [Chemical Formula 2-1] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-1] and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-1] and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of 1,4-dioxane, and bis(pinacolato)diboron, Pd(dppf)Cl₂, and potassium acetate were added, and the mixture was stirred and reacted at 100° C. for 10 hours to produce a third reaction product. At this time, bis(pinacolato)diboron was added so that the compound represented by [Chemical Formula 2-1] and bis(pinacolato)diboron were in a molar ratio of 1:7.0; Pd(dppf)Cl₂ was added so that the compound represented by [Chemical Formula 2-1] and Pd(dppf)Cl₂ were in a molar ratio of 1:0.06; and potassium acetate was added so that the compound represented by [Chemical Formula 2-1] and potassium acetate were in a molar ratio of 1:6.0.
(8) The prepared third reaction product was extracted with dichloromethane, and the organic phase of the extract was concentrated under reduced pressure to prepare a concentrate. Thereafter, the concentrate was purified through silica gel chromatography to manufacture a compound having an unshared electron pair for a perovskite solar cell represented by [Chemical Formula 1-6] below.

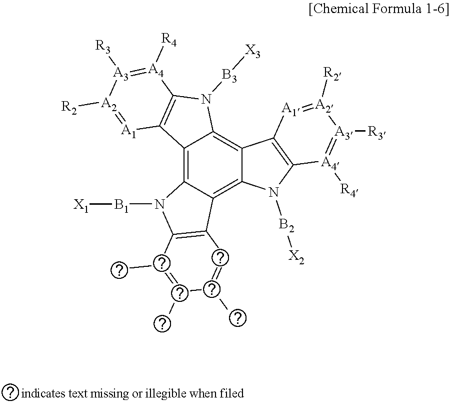

In [Chemical Formula 1-6], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), R₂, R₃, R₄, R_{2′}, R_{3′}, R_{4′}, R_{2″}, R_{3″}, and R_{4″} are each —H, B₁, B₂, and B₃ are each

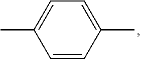

X₁, X₂, and X₃ are each

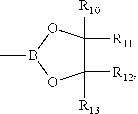

and R₁₀, R₁₁, R₁₂, and R¹³ are each a methyl group.

### Example 1-7: Preparation of a Compound Having an Unshared Electron Pair for a Perovskite Solar Cell

(1) Under an argon atmosphere, 223.65 mmol of the compound represented by [Chemical Formula 3-2] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-2].
(4) The compound represented by [Chemical Formula 2-2] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-2] and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-2] and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of 1,4-dioxane, and bis(pinacolato)diboron, Pd(dppf)Cl₂, and potassium acetate were added, and the mixture was stirred and reacted at 100° C. for 10 hours to produce a third reaction product. At this time, bis(pinacolato)diboron was added so that the compound represented by [Chemical Formula 2-2] and bis(pinacolato)diboron were in a molar ratio of 1:7.0; Pd(dppf)Cl₂ was added so that the compound represented by [Chemical Formula 2-2] and Pd(dppf)Cl₂ were in a molar ratio of 1:0.06; and potassium acetate was added so that the compound represented by [Chemical Formula 2-2] and potassium acetate were in a molar ratio of 1:6.0.
(8) The prepared third reaction product was extracted with dichloromethane, and the organic phase of the extract was concentrated under reduced pressure to prepare a concentrate. Thereafter, the concentrate was purified through silica gel chromatography to manufacture a compound having an unshared electron pair for a perovskite solar cell represented by [Chemical Formula 1-7] below.

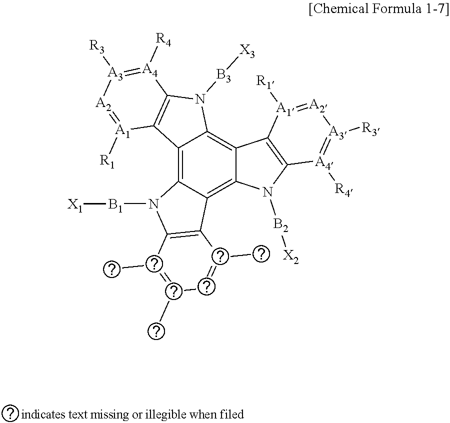

In [Chemical Formula 1-7], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), R₁, R₃, R₄, R_{1′}, R_{3′}, R_{4′}, R_{1″}, R_{3″}, and R_{4″} are each —H, B₁, B₂, and B₃ are each

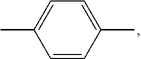

X₁, X₂, and X₃ are each

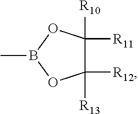

and R₁₀, R₁₁, R₁₂, and R¹³ are each a methyl group.

### Example 1-8: Preparation of a Compound Having an Unshared Electron Pair for a Perovskite Solar Cell

(1) Under an argon atmosphere, 223.65 mmol of the compound represented by [Chemical Formula 3-3] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-3].
(4) The compound represented by [Chemical Formula 2-3] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-3] and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-3] and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of 1,4-dioxane, and bis(pinacolato)diboron, Pd(dppf)Cl₂, and potassium acetate were added, and the mixture was stirred and reacted at 100° C. for 10 hours to produce a third reaction product. At this time, bis(pinacolato)diboron was added so that the compound represented by [Chemical Formula 2-3] and bis(pinacolato)diboron were in a molar ratio of 1:7.0; Pd(dppf)Cl₂ was added so that the compound represented by [Chemical Formula 2-3] and Pd(dppf)Cl₂ were in a molar ratio of 1:0.06; and potassium acetate was added so that the compound represented by [Chemical Formula 2-3] and potassium acetate were in a molar ratio of 1:6.0.
(8) The prepared third reaction product was extracted with dichloromethane, and the organic phase of the extract was concentrated under reduced pressure to prepare a concentrate. Thereafter, the concentrate was purified through silica gel chromatography to manufacture a compound having an unshared electron pair for a perovskite solar cell represented by [Chemical Formula 1-8] below.

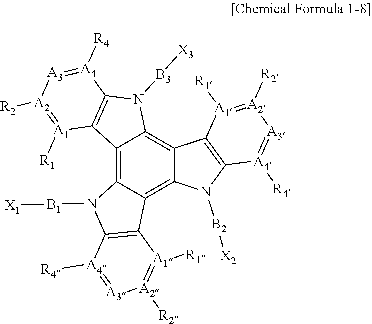

In [Chemical Formula 1-8], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), R₁, R₂, R₄, R_{1′}, R_{2′}, R_{4′}, R_{1″}, R_{2″}, and R_{4″} are each —H, B₁, B₂, and B₃ are each

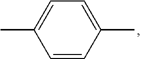

X₁, X₂, and X₃ are each

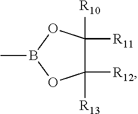

and R₁₀, R₁₁, R₁₂, and R¹³ are each a methyl group.

### Example 1-9: Preparation of a Compound Having an Unshared Electron Pair for a Perovskite Solar Cell

(1) Under an argon atmosphere, 223.65 mmol of the compound represented by [Chemical Formula 3-4] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained 1 h silica gel column chromatography (eluent: dichloromethane) to prepare a purified product.
(3) The prepared purified product was concentrated under reduced pressure and recrystallized using acetone to prepare the compound represented by [Chemical Formula 2-4].
(4) The compound represented by [Chemical Formula 2-4] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-4] and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-4] and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of 1,4-dioxane, and bis(pinacolato)diboron, Pd(dppf)Cl₂, and potassium acetate were added, and the mixture was stirred and reacted at 100° C. for 10 hours to produce a third reaction product. At this time, bis(pinacolato)diboron was added so that the compound represented by [Chemical Formula 2-4] and bis(pinacolato)diboron were in a molar ratio of 1:7.0; Pd(dppf)Cl₂ was added so that the compound represented by [Chemical Formula 2-4] and Pd(dppf)Cl₂ were in a molar ratio of 1:0.06; and potassium acetate was added so that the compound represented by [Chemical Formula 2-4] and potassium acetate were in a molar ratio of 1:6.0.
(8) The prepared third reaction product was extracted with dichloromethane, and the organic phase of the extract was concentrated under reduced pressure to prepare a concentrate. Thereafter, the concentrate was purified through silica gel chromatography to manufacture a compound having an unshared electron pair for a perovskite solar cell represented by [Chemical Formula 1-9] below.

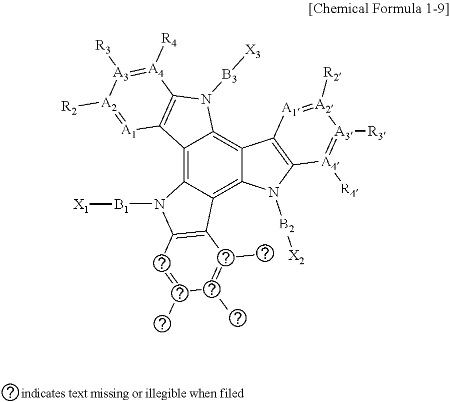

In [Chemical Formula 1-9], A₁, A₂, A₃, A₁, A_{2′}, A_{3′}, A_{1″}, A_{2″}, and A_{3″} are each carbon (C), A₄, A_{4′}, and A_{4″} are each nitrogen (N), R₁, R₂, R₃, R_{1′}, R_{2′}, R_{3′}, R_{1″}, R_{2″}, and R_{3″} are each —H, B₁, B₂, and B₃ are each

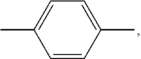

X₁, X₂, and X₃ are each

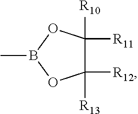

and R₁₀, R₁₁, R₁₂, and R¹³ are each a methyl group.

### Example 2-1: Preparation of a Compound Having an Unshared Electron Pair for a Perovskite Solar Cell

(1) Under an argon atmosphere, 223.65 mmol of a compound represented by [Chemical Formula 3-1] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-1] below.
(4) The prepared compound represented by [Chemical Formula 2-1] below was dissolved in 300 ml of anhydrous dimethyl formamide, sodium hydride was added, and the mixture was stirred at 20° C. for 10 minutes to prepare a mixture. At this time, the compound represented by [Chemical Formula 2-1] below and sodium hydride were fed in a molar ratio of 1:3.
(5) diethyl(3-bromopropyl)phosphonate was added to the prepared mixture, and the mixture was stirred at 100° C. for 10 hours to produce a second reaction product. At this time, diethyl(3-bromopropyl)phosphonate was added so that the compound represented by [Chemical Formula 2-1] below and diethyl(3-bromopropyl)phosphonate were in a molar ratio of 1:3.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in dichloromethane, and bromotrimethylsilane was added. The mixture was stirred and reacted at 20° C. for 24 hours to produce a third reaction product. The third reaction product thus obtained was recrystallized using methanol to manufacture a compound having an unshared electron pair for a perovskite solar cell represented by [Chemical Formula 1-10] below. At this time, bromotrimethylsilane was added so that the compound represented by [Chemical Formula 2-1] below and bromotrimethylsilane were in a molar ratio of 1:5.

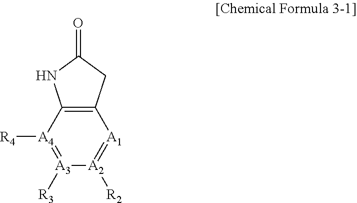

In [Chemical Formula 3-1], A₂, A₃, and A₄ are each carbon (C), A₁ is nitrogen (N), and R₂, R₃, and R₄ are each —H.

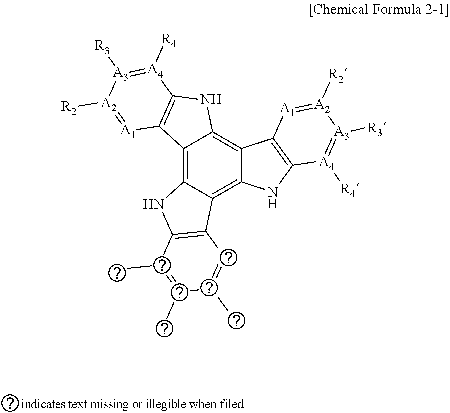

In [Chemical Formula 2-1], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), and R₂, R₃, R₄, R_{2′}, R_{3′}, R_{4′}, R_{2″}, R_{3″}, and R_{4″} are each —H.

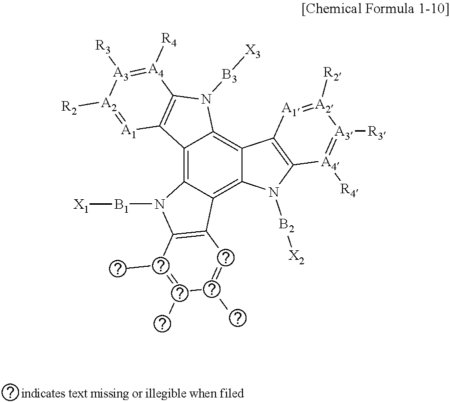

In [Chemical Formula 1-10], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), R₂, R₃, R₄, R_{2′}, R_{3′}, R_{4′}, R_{2″}, R_{3″}, and R_{4″} are each —H, B₁, B₂, and B₃ are each —CH₂CH₂CH₂—, and X₁, X₂, and X₃ are each a dihydroxyphosphoryl group (—P═O(OH)₂).

### Example 2-2: Preparation of a Compound Having an Unshared Electron Pair for a Perovskite Solar Cell

(1) Under an argon atmosphere, 223.65 mmol of a compound represented by [Chemical Formula 3-2] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was h silica gel column chromatography (eluent: dichloromethane) to prepare a purified product.
(3) The prepared purified product was concentrated under reduced pressure and recrystallized using acetone to prepare a compound represented by [Chemical Formula 2-1] below.
(4) The prepared compound represented by [Chemical Formula 2-2] below was dissolved in 300 ml of anhydrous dimethyl formamide, sodium hydride was added, and the mixture was stirred at 20° C. for 10 minutes to prepare a mixture. At this time, the compound represented by [Chemical Formula 2-2] below and sodium hydride were fed in a molar ratio of 1:3.
(5) diethyl(3-bromopropyl)phosphonate was added to the prepared mixture, and the mixture was stirred at 100° C. for 10 hours to produce a second reaction product. At this time, diethyl(3-bromopropyl)phosphonate was added so that the compound represented by [Chemical Formula 2-2] below and diethyl(3-bromopropyl)phosphonate were in a molar ratio of 1:3.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in dichloromethane, and bromotrimethylsilane was added. The mixture was stirred and reacted at 20° C. for 24 hours to produce a third reaction product. The third reaction product thus obtained was recrystallized using methanol to manufacture a compound having an unshared electron pair for a perovskite solar cell represented by [Chemical Formula 1-11] below. At this time, bromotrimethylsilane was added so that the compound represented by [Chemical Formula 2-2] below and bromotrimethylsilane were in a molar ratio of 1:5.

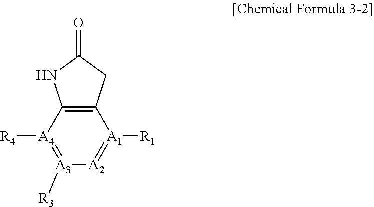

In [Chemical Formula 3-2], A₁, A₃, and A₄ are each carbon (C), A₂ is nitrogen (N), and R₁, R₃, and R₄ are each —H.

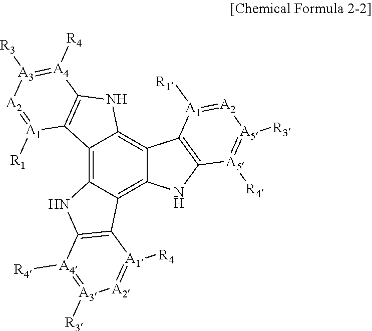

In [Chemical Formula 2-2], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), and R₁, R₃, R₄, R_{1′}, R_{3′}, R_{4′}, R_{1″}, R_{3″}, and R_{4″} are each —H.

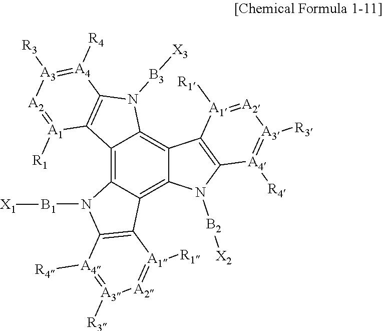

In [Chemical Formula 1-11], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), R₁, R₃, R₄, R_{1′}, R_{3′}, R_{4′}, R_{1″}, R_{3″}, and R_{4″} are each —H, B₁, B₂, and B₃ are each —CH₂CH₂CH₂—, and X₁, X₂, and X₃ are each a dihydroxyphosphoryl group (—P═O(OH)₂).

### Example 2-3: Preparation of a Compound Having an Unshared Electron Pair for a Perovskite Solar Cell

(1) Under an argon atmosphere, 223.65 mmol of a compound represented by [Chemical Formula 3-3] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-3] below.
(4) The prepared compound represented by [Chemical Formula 2-3] below was dissolved in 300 ml of anhydrous dimethyl formamide, sodium hydride was added, and the mixture was stirred at 20° C. for 10 minutes to prepare a mixture. At this time, the compound represented by [Chemical Formula 2-3] below and sodium hydride were added at a molar ratio of 1:3.
(5) diethyl(3-bromopropyl)phosphonate was added to the prepared mixture, and the mixture was stirred at 100° C. for 10 hours to produce a second reaction product. At this time, diethyl(3-bromopropyl)phosphonate was added so that the compound represented by [Chemical Formula 2-3] below and diethyl(3-bromopropyl)phosphonate were in a molar ratio of 1:3.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in dichloromethane, and bromotrimethylsilane was added. The mixture was stirred and reacted at 20° C. for 24 hours to produce a third reaction product. The third reaction product thus obtained was recrystallized using methanol to manufacture a compound having an unshared electron pair for a perovskite solar cell represented by [Chemical Formula 1-12] below. At this time, bromotrimethylsilane was added so that the compound represented by [Chemical Formula 2-3] below and bromotrimethylsilane were in a molar ratio of 1:5.

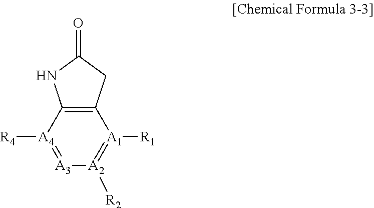

In [Chemical Formula 3-3], A₁, A₂, and A₄ are each carbon (C), A₃ is nitrogen (N), and R₁, R₂, and R₄ are each —H.

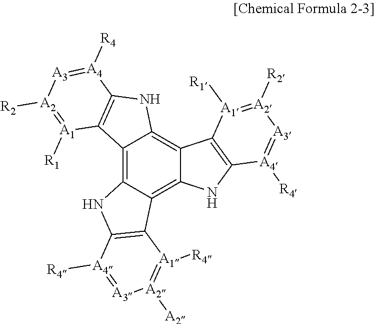

In [Chemical Formula 2-3], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), and R₁, R₂, R₄, R_{1′}, R_{2′}, R_{4′}, R_{1″}, R_{2″}, and R_{4″} are each —H.

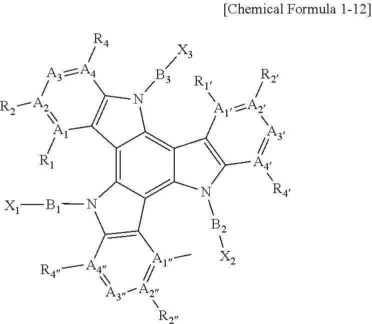

In [Chemical Formula 1-12], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), R₁, R₂, R₄, R_{1′}, R_{2′}, R_{4′}, R_{1″}, R_{2″}, and R_{4″} are each —H, B₁, B₂, and B₃ are each —CH₂CH₂CH₂—, and X₁, X₂, and X₃ are each a dihydroxyphosphoryl group (—P═O(OH)₂).

### Example 2-4: Preparation of a Compound Having an Unshared Electron Pair for a Perovskite Solar Cell

(1) Under an argon atmosphere, 223.65 mmol of a compound represented by [Chemical Formula 3-4] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified d through silica gel column chromatography (eluent: dichloromethane) to prepare a purified product.
(3) The prepared purified product was concentrated under reduced pressure and recrystallized using acetone to prepare a compound represented by [Chemical Formula 2-4] below.
(4) The prepared compound represented by [Chemical Formula 2-4] below was dissolved in 300 ml of anhydrous dimethyl formamide, sodium hydride was added, and the mixture was stirred at 20° C. for 10 minutes to prepare a mixture. At this time, the compound represented by [Chemical Formula 2-4] below and sodium hydride were fed in a molar ratio of 1:3.
(5) diethyl(3-bromopropyl)phosphonate was added to the prepared mixture, and the mixture was stirred at 100° C. for 10 hours to produce a second reaction product. At this time, diethyl(3-bromopropyl)phosphonate was added so that the compound represented by [Chemical Formula 2-4] below and diethyl(3-bromopropyl)phosphonate were in a molar ratio of 1:3.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in dichloromethane, and bromotrimethylsilane was added. The mixture was stirred and reacted at 20° C. for 24 hours to produce a third reaction product. The third reaction product thus obtained was recrystallized using methanol to manufacture a compound having an unshared electron pair for a perovskite solar cell represented by [Chemical Formula 1-13] below. At this time, bromotrimethylsilane was added so that the compound represented by [Chemical Formula 2-4] below and bromotrimethylsilane were in a molar ratio of 1:5.

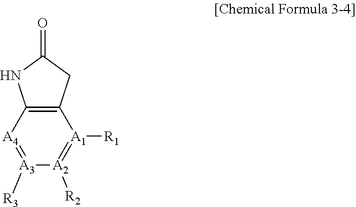

In [Chemical Formula 3-4], A₁, A₂, and A₃ are each carbon (C), A₄ is nitrogen (N), and R₁, R₂, and R₃ are each —H.

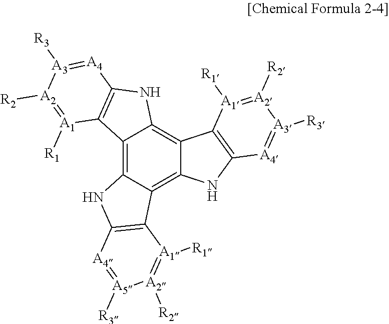

In [Chemical Formula 2-4], A₁, A₂, A₃, A_{1′}, A_{2′}, A_{3′}, A_{1″}, A_{2″}, and A_{3″} are each carbon (C), A₄, A_{4′}, and A_{4″} are each nitrogen (N), and R₁, R₂, R₃, R_{1′}, R_{2′}, R_{3′}, R_{1″}, R_{2″}, and R_{3″} are each —H.

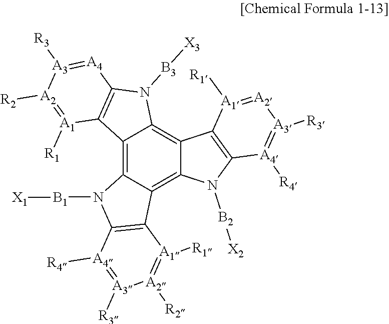

In [Chemical Formula 1-13], A₁, A₂, A₃, A_{1′}, A_{2′}, A_{3′}, A_{1″}, A_{2″}, and A_{3″} are each carbon (C), A₄, A_{4′}, and A_{4″} are each nitrogen (N), R₁, R₂, R₃, R_{1′}, R_{2′}, R_{3′}, R_{1″}, R_{2″}, and R_{3″} are each —H, B₁, B₂, and B₃ are each —CH₂CH₂CH₂—, and X₁, X₂, and X₃ are each a dihydroxyphosphoryl group (—P═O(OH)₂).

### Example 2-5: Preparation of a Compound Having an Unshared Electron Pair for a Perovskite Solar Cell

(1) Under an argon atmosphere, 223.65 mmol of the compound represented by [Chemical Formula 3-1] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-1].
(4) The compound represented by [Chemical Formula 2-1] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-1] and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-1] and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous toluene, and diethyl phosphite, triphenylphosphine, and cesium carbonate were added. The mixture was stirred and reacted at 110° C. for 12 hours to produce a third reaction product. At this time, diethyl phosphite was added such that the compound represented by [Chemical Formula 2-1] and diethyl phosphite were in a molar ratio of 1:3.3; triphenylphosphine was added such that the compound represented by [Chemical Formula 2-1] and triphenylphosphine were in a molar ratio of 1:0.12; and cesium carbonate was added such that the compound represented by [Chemical Formula 2-1] and cesium carbonate were in a molar ratio of 1:3.
(8) The prepared third reaction product was filtered to remove solid impurities, and the organic phase of the third reaction product was concentrated under reduced pressure to prepare a concentrate. Thereafter, 100 ml of a 6N hydrochloric acid (HCl) solution (containing 100 mmol of HCl) was added to the concentrate, and the mixture was stirred at 80° C. for 4 hours. The resulting solid was filtered and washed to manufacture a compound having an unshared electron pair for a perovskite solar cell represented by [Chemical Formula 1-14] below. At this time, hydrochloric acid was added such that the compound represented by [Chemical Formula 2-1] and hydrochloric acid were in a molar ratio of 1:7.

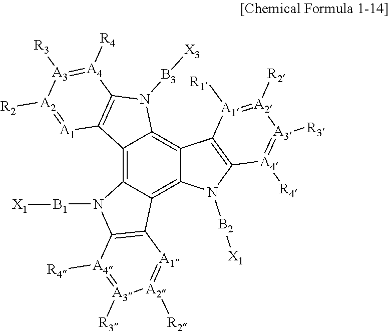

In [Chemical Formula 1-14], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), R₂, R₃, R₄, R_{2′}, R_{3′}, R_{4′}, R_{2″}, R_{3″}, and R_{4″} are each —H, B₁, B₂, and B₃ are each

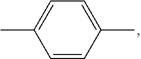

and X₁, X₂, and X₃ are each a dihydroxyphosphoryl group (—P═O(OH)₂).

### Example 2-6: Preparation of a Compound Having an Unshared Electron Pair for a Perovskite Solar Cell

(1) Under an argon atmosphere, 223.65 mmol of the compound represented by [Chemical Formula 3-2] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-2].
(4) The compound represented by [Chemical Formula 2-2] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-2] and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-2] and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous toluene, and diethyl phosphite, triphenylphosphine, and cesium carbonate were added. The mixture was stirred and reacted at 110° C. for 12 hours to produce a third reaction product. At this time, diethyl phosphite was added such that the compound represented by [Chemical Formula 2-2] and diethyl phosphite were in a molar ratio of 1:3.3; triphenylphosphine was added such that the compound represented by [Chemical Formula 2-2] and triphenylphosphine were in a molar ratio of 1:0.12; and cesium carbonate was added such that the compound represented by [Chemical Formula 2-2] and cesium carbonate were in a molar ratio of 1:3.
(8) The prepared third reaction product was filtered to remove solid impurities, and the organic phase of the third reaction product was concentrated under reduced pressure to prepare a concentrate. Thereafter, 100 ml of a 6N hydrochloric acid (HCl) solution (containing 100 mmol of HCl) was added to the concentrate, and the mixture was stirred at 80° C. for 4 hours. The resulting solid was filtered and washed to manufacture a compound having an unshared electron pair for a perovskite solar cell represented by [Chemical Formula 1-15] below. At this time, hydrochloric acid was added such that the compound represented by [Chemical Formula 2-2] and hydrochloric acid were in a molar ratio of 1:7.

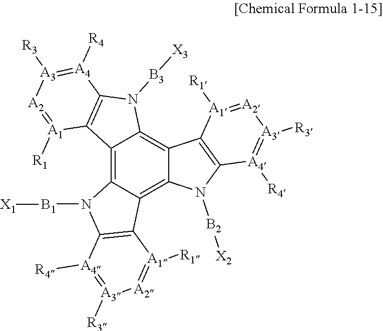

In [Chemical Formula 1-15], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), R₁, R₃, R₄, R_{1′}, R_{3′}, R_{4′}, R_{1″}, R_{3″}, and R_{4″} are each —H, B₁, B₂, and B₃ are each

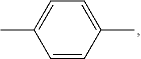

and X₁, X₂, and X₃ are each a dihydroxyphosphoryl group (—P═O(OH)₂).

### Example 2-7: Preparation of a Compound Having an Unshared Electron Pair for a Perovskite Solar Cell

(1) Under an argon atmosphere, 223.65 mmol of the compound represented by [Chemical Formula 3-3] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-3].
(4) The compound represented by [Chemical Formula 2-3] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-3] and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-3] and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous toluene, and diethyl phosphite, triphenylphosphine, and cesium carbonate were added. The mixture was stirred and reacted at 110° C. for 12 hours to produce a third reaction product. At this time, diethyl phosphite was added such that the compound represented by [Chemical Formula 2-3] and diethyl phosphite were in a molar ratio of 1:3.3; triphenylphosphine was added such that the compound represented by [Chemical Formula 2-3] and triphenylphosphine were in a molar ratio of 1:0.12; and cesium carbonate was added such that the compound represented by [Chemical Formula 2-3] and cesium carbonate were in a molar ratio of 1:3.
(8) The prepared third reaction product was filtered to remove solid impurities, and the organic phase of the third reaction product was concentrated under reduced pressure to prepare a concentrate. Thereafter, 100 ml of a 6N hydrochloric acid (HCl) solution (containing 100 mmol of HCl) was added to the concentrate, and the mixture was stirred at 80° C. for 4 hours. The resulting solid was filtered and washed to manufacture a compound having an unshared electron pair for a perovskite solar cell represented by [Chemical Formula 1-16] below. At this time, hydrochloric acid was added such that the compound represented by [Chemical Formula 2-3] and hydrochloric acid were in a molar ratio of 1:7.

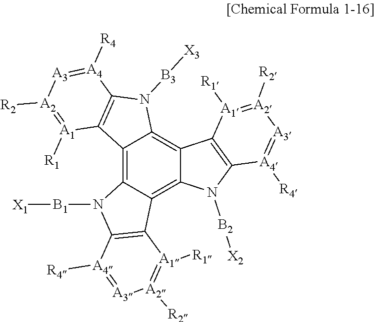

In [Chemical Formula 1-16], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), R₁, R₂, R₄, R_{1′}, R_{2′}, R_{4′}, R_{1″}, R_{2″}, and R_{4″} are each —H, B₁, B₂, and B₃ are each

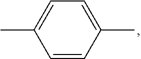

and X₁, X₂, and X₃ are each a dihydroxyphosphoryl group (—P═O(OH)₂).

### Example 2-8: Preparation of a Compound Having an Unshared Electron Pair for a Perovskite Solar Cell

(1) Under an argon atmosphere, 223.65 mmol of the compound represented by [Chemical Formula 3-4] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-3].
(4) The compound represented by [Chemical Formula 2-4] prepared was dissolved in 50 ml of anhydrous dimethyl formamide, sodium hydride was added, and the mixture was stirred at 25° C. for 10 minutes to prepare a mixture. At this time, the compound represented by [Chemical Formula 2-4] and sodium hydride were added at a molar ratio of 1:3.1.
(5) 1,4-dibromobenzene was added to the prepared mixture, and the mixture was stirred at 100° C. for 10 hours to produce a second reaction product. At this time, 1,4-dibromobenzene was added so that the compound represented by [Chemical Formula 2-4] and 1,4-dibromobenzene were in a molar ratio of 1:3.1.
(6) The prepared second reaction product was extracted with dichloromethane, and the organic phase of the extract was concentrated under reduced pressure to prepare a concentrate. Then, the concentrate was purified through silica gel chromatography and dried to obtain a dried product.
(7) The obtained dried product was dissolved in 300 ml of anhydrous toluene, and diethyl phosphite, triphenylphosphine, and cesium carbonate were added. The mixture was stirred and reacted at 110° C. for 12 hours to produce a third reaction product. At this time, diethyl phosphite was added such that the compound represented by [Chemical Formula 2-4] and diethyl phosphite were in a molar ratio of 1:3.3; triphenylphosphine was added such that the compound represented by [Chemical Formula 2-4] and triphenylphosphine were in a molar ratio of 1:0.12; and cesium carbonate was added such that the compound represented by [Chemical Formula 2-4] and cesium carbonate were in a molar ratio of 1:3.
(8) The prepared third reaction product was filtered to remove solid impurities, and the organic phase of the third reaction product was concentrated under reduced pressure to prepare a concentrate. Thereafter, 100 ml of a 6N hydrochloric acid (HCl) solution (containing 100 mmol of HCl) was added to the concentrate, and the mixture was stirred at 80° C. for 4 hours. The resulting solid was filtered and washed to manufacture a compound having an unshared electron pair for a perovskite solar cell represented by [Chemical Formula 1-17] below. At this time, hydrochloric acid was added such that the compound represented by [Chemical Formula 2-4] and hydrochloric acid were in a molar ratio of 1:7.

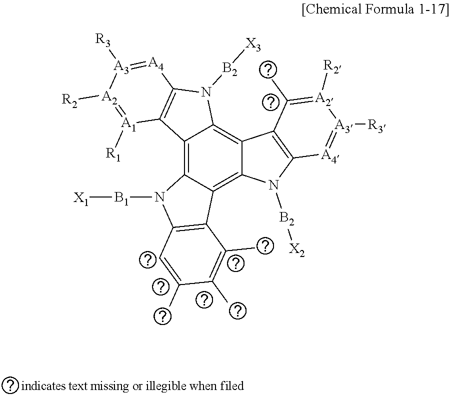

In [Chemical Formula 1-17], A₁, A₂, A₃, A₁, A_{2′}, A_{3′}, A_{1″}, A_{2″}, and A_{3″} are each carbon (C), A₄, A_{4′}, and A_{4″} are each nitrogen (N), R₁, R₂, R₃, R_{1′}, R_{2′}, R_{3′}, R_{1″}, R_{2″}, and R_{3″} are each —H, B₁, B₂, and B₃ are each

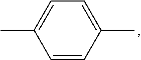

and X₁, X₂, and X₃ are each a dihydroxyphosphoryl group (—P═O(OH)₂).

### Example 3-1: Preparation of a Compound Having an Unshared Electron Pair for a Perovskite Solar Cell

(1) Under an argon atmosphere, 223.65 mmol of a compound represented by [Chemical Formula 3-1] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-1] below.
(4) The prepared compound represented by [Chemical Formula 2-1] below was dissolved in 50 ml of anhydrous dimethyl formamide, and ethyl 3-bromopropionate, potassium carbonate, and potassium iodide were added. The mixture was stirred at 110° C. for 10 hours to prepare a second reaction product. At this time, ethyl 3-bromopropionate was added such that the compound represented by [Chemical Formula 2-1] below and ethyl 3-bromopropionate were in a molar ratio of 1:3.1; potassium carbonate was added such that the compound represented by [Chemical Formula 2-1] below and potassium carbonate were in a molar ratio of 1:6; and potassium iodide was added such that the compound represented by [Chemical Formula 2-1] below and potassium iodide were in a molar ratio of 1:0.2.
(5) The prepared second reaction product was extracted with dichloromethane, and the organic phase of the extract was concentrated under reduced pressure to prepare a concentrate. Then, the concentrate was purified through silica gel chromatography and dried to obtain a dried product.
(6) The obtained dried product was dissolved in a mixed solvent containing ethanol and water, and a 2N sodium hydroxide (NaOH) solution (containing 100 mmol of NaOH) was added. The mixture was stirred and reacted at 80° C. for 12 hours to prepare a third reaction product. The third reaction product thus obtained was filtered to manufacture a compound having an unshared electron pair for a perovskite solar cell represented by [Chemical Formula 1-18] below. At this time, sodium hydroxide (NaOH) was added so that the compound represented by [Chemical Formula 2-1] below and sodium hydroxide were in a molar ratio of 1:7.

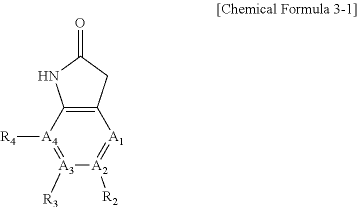

In [Chemical Formula 3-1], A₂, A₃, and A₄ are each carbon (C), A₁ is nitrogen (N), and R₂, R₃, and R₄ are each —H.

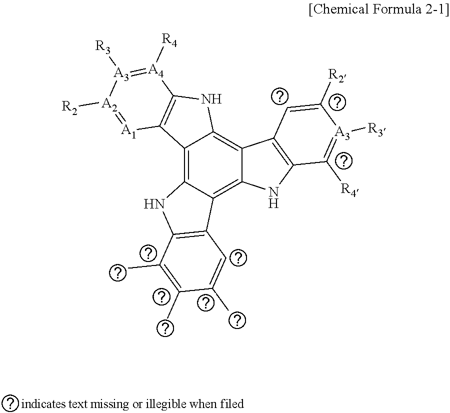

In [Chemical Formula 2-1], A₂, A₃, A₄, A₂, A_{3′}, A_{4′}, A_{2″}, A_{3″}, and A_{4″} are each carbon (C), A₁, A_{1′}, and A_{1″} are each nitrogen (N), and R₂, R₃, R₄, R_{2′}, R_{3′}, R_{4′}, R_{2″}, R_{3″}, and R_{4″} are each —H.

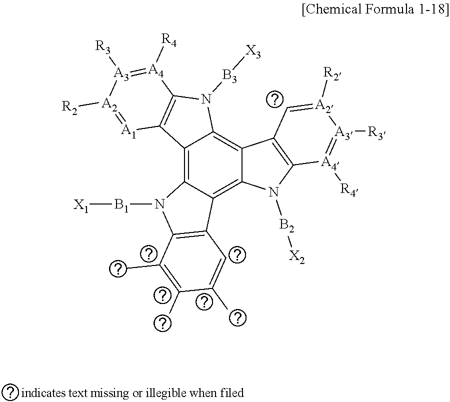

In [Chemical Formula 1-18], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), R₂, R₃, R₄, R_{2′}, R_{3′}, R_{4′}, R_{2″}, R_{3″}, and R_{4″} are each —H, B₁, B₂, and B₃ are each —CH₂CH₂CH₂—, and X₁, X₂, and X₃ are each a carboxyl group (—COOH).

### Example 3-2: Preparation of a Compound Having an Unshared Electron Pair for a Perovskite Solar Cell

(1) Under an argon atmosphere, 223.65 mmol of a compound represented by [Chemical Formula 3-2] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-1] below.
(4) The prepared compound represented by [Chemical Formula 2-2] below was dissolved in 50 ml of anhydrous dimethyl formamide, and ethyl 3-bromopropionate, potassium carbonate, and potassium iodide were added. The mixture was stirred at 110° C. for 10 hours to prepare a second reaction product. At this time, ethyl 3-bromopropionate was added such that the compound represented by [Chemical Formula 2-2] below and ethyl 3-bromopropionate were in a molar ratio of 1:3.1; potassium carbonate was added such that the compound represented by [Chemical Formula 2-2] below and potassium carbonate were in a molar ratio of 1:6; and potassium iodide was added such that the compound represented by [Chemical Formula 2-2] below and potassium iodide were in a molar ratio of 1:0.2.
(5) The prepared second reaction product was extracted with dichloromethane, and the organic phase of the extract was concentrated under reduced pressure to prepare a concentrate. Then, the concentrate was purified through silica gel chromatography and dried to obtain a dried product.
(6) The obtained dried product was dissolved in a mixed solvent containing ethanol and water, and a 2N sodium hydroxide (NaOH) solution (containing 100 mmol of NaOH) was added. The mixture was stirred and reacted at 80° C. for 12 hours to prepare a third reaction product. The third reaction product thus obtained was filtered to manufacture a compound having an unshared electron pair for a perovskite solar cell represented by [Chemical Formula 1-19] below. At this time, sodium hydroxide (NaOH) was added so that the compound represented by [Chemical Formula 2-2] below and sodium hydroxide were in a molar ratio of 1:7.

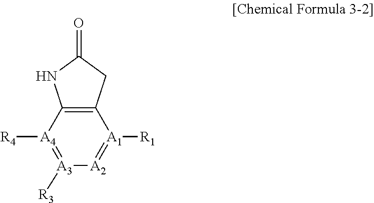

In [Chemical Formula 3-2], A₁, A₃, and A₄ are each carbon (C), A₂ is nitrogen (N), and R₁, R₃, and R₄ are each —H.

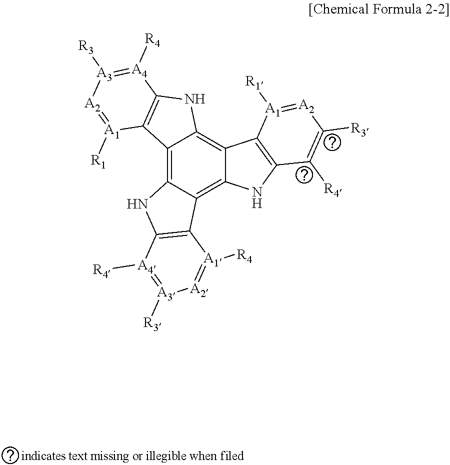

In [Chemical Formula 2-2], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), and R₁, R₃, R₄, R_{1′}, R_{3′}, R_{4′}, R_{1″}, R_{3″}, and R_{4″} are each —H.

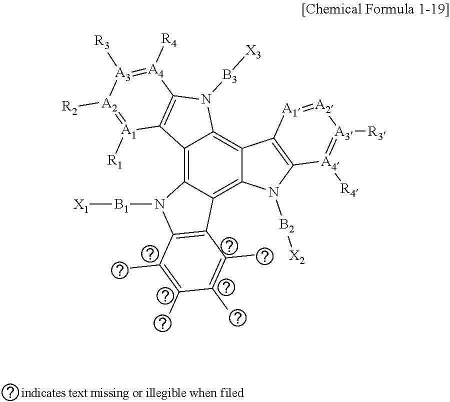

In [Chemical Formula 1-19], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), R₁, R₃, R₄, R_{1′}, R_{3′}, R_{4′}, R_{1″}, R_{3″}, and R_{4″} are each —H, B₁, B₂, and B₃ are each —CH₂CH₂CH₂—, and X₁, X₂, and X₃ are each a carboxyl group (—COOH).

### Example 3-3: Preparation of a Compound Having an Unshared Electron Pair for a Perovskite Solar Cell

(1) Under an argon atmosphere, 223.65 mmol of a compound represented by [Chemical Formula 3-3] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-3] below.
(4) The prepared compound represented by [Chemical Formula 2-3] below was dissolved in 50 ml of anhydrous dimethyl formamide, and ethyl 3-bromopropionate, potassium carbonate, and potassium iodide were added. The mixture was stirred at 110° C. for 10 hours to prepare a second reaction product. At this time, ethyl 3-bromopropionate was added such that the compound represented by [Chemical Formula 2-3] below and ethyl 3-bromopropionate were in a molar ratio of 1:3.1; potassium carbonate was added such that the compound represented by [Chemical Formula 2-3] below and potassium carbonate were in a molar ratio of 1:6; and potassium iodide was added such that the compound represented by [Chemical Formula 2-3] below and potassium iodide were in a molar ratio of 1:0.2.
(5) The prepared second reaction product was extracted with dichloromethane, and the organic phase of the extract was concentrated under reduced pressure to prepare a concentrate. Then, the concentrate was purified through silica gel chromatography and dried to obtain a dried product.
(6) The obtained dried product was dissolved in a mixed solvent containing ethanol and water, and a 2N sodium hydroxide (NaOH) solution (containing 100 mmol of NaOH) was added. The mixture was stirred and reacted at 80° C. for 12 hours to prepare a third reaction product. The third reaction product thus obtained was filtered to manufacture a compound having an unshared electron pair for a perovskite solar cell represented by [Chemical Formula 1-20] below. At this time, sodium hydroxide (NaOH) was added so that the compound represented by [Chemical Formula 2-3] below and sodium hydroxide were in a molar ratio of 1:7.

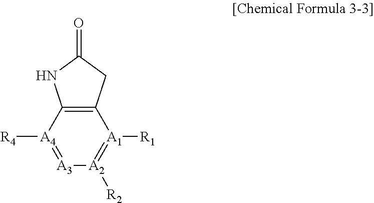

In [Chemical Formula 3-3], A₁, A₂, and A₄ are each carbon (C), A₃ is nitrogen (N), and R₁, R₂, and R₄ are each —H.

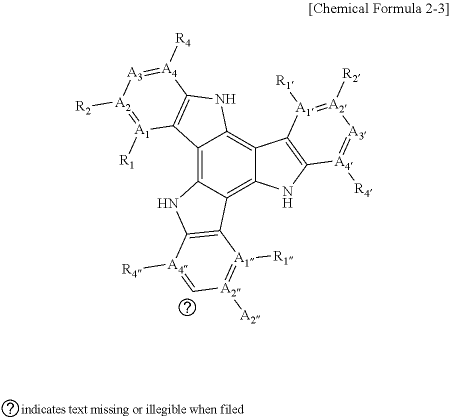

In [Chemical Formula 2-3], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), and R₁, R₂, R₄, R_{1′}, R_{2′}, R_{4′}, R_{1″}, R_{2″}, and R_{4″} are each —H.

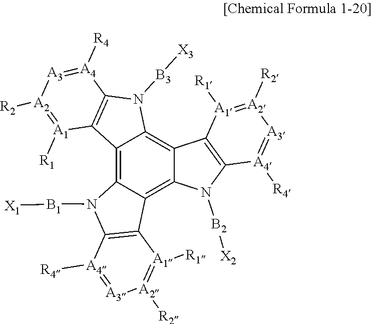

In [Chemical Formula 1-20], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), R₁, R₂, R₄, R_{1′}, R_{2′}, R_{4′}, R_{1″}, R_{2″}, and R_{4″} are each —H, B₁, B₂, and B₃ are each —CH₂CH₂CH₂—, and X₁, X₂, and X₃ are each a carboxyl group (—COOH).

### Example 4-1: Preparation of a Compound Having an Unshared Electron Pair for a Perovskite Solar Cell

(1) Under an argon atmosphere, 223.65 mmol of a compound represented by [Chemical Formula 3-1] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-1] below.
(4) The prepared compound represented by [Chemical Formula 2-1] below was dissolved in 50 ml of anhydrous dimethyl formamide, and 1,3-dibromopropane, potassium carbonate, and potassium iodide were added. The mixture was stirred at 110° C. for 10 hours to prepare a second reaction product. At this time, 1,3-dibromopropane was added such that the compound represented by [Chemical Formula 2-1] below and 1,3-dibromopropane were in a molar ratio of 1:3.3; potassium carbonate was added such that the compound represented by [Chemical Formula 2-1] below and potassium carbonate were in a molar ratio of 1:6; and potassium iodide was added such that the compound represented by [Chemical Formula 2-1] below and potassium iodide were in a molar ratio of 1:0.2.
(5) The prepared second reaction product was extracted with dichloromethane, and the organic phase of the extract was concentrated under reduced pressure to prepare a concentrate. Then, the concentrate was purified through silica gel chromatography and dried to obtain a dried product.
(6) The obtained dried product was dissolved in a mixed solvent containing ethanol and water, and sodium sulfite was added. The mixture was stirred and reacted at 80° C. for 6 hours to prepare a third reaction product. At this time, sodium sulfite was added such that the compound represented by [Chemical Formula 2-1] below and sodium sulfite were in a molar ratio of 1:3.3.
(7) The third reaction product thus obtained was cooled to 25° C., and a 6 N hydrochloric acid (HCl) solution (containing 100 mmol of HCl) was added and stirred to prepare a fourth reaction product. The obtained fourth reaction product was then filtered to manufacture a compound having an unshared electron pair for a perovskite solar cell represented by [Chemical Formula 1-21] below. At this time, hydrochloric acid (HCl) was added such that the compound represented by [Chemical Formula 2-1] below and hydrochloric acid were in a molar ratio of 1:7.

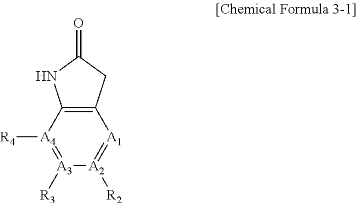

In [Chemical Formula 3-1], A₂, A₃, and A₄ are each carbon (C), A₁ is nitrogen (N), and R₂, R₃, and R₄ are each —H.

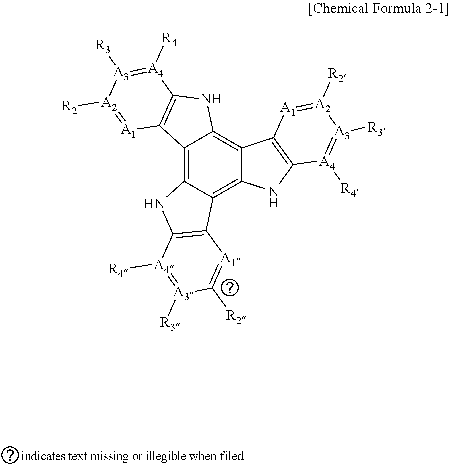

In [Chemical Formula 2-1], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), and R₂, R₃, R₄, R_{2′}, R_{3′}, R_{4′}, R_{2″}, R_{3″}, and R_{4″} are each —H.

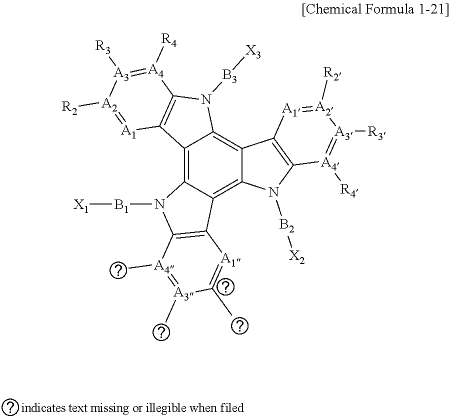

In [Chemical Formula 1-21], A₂, A₃, A₄, A_{2′}, A_{3′}, A_{4′}, A_{2″}, A_{3″}, and A_{4″} are each carbon (C), A₁, A_{1′}, and A_{1″} are each nitrogen (N), R₂, R₃, R₄, R_{2′}, R_{3′}, R_{4′}, R_{2″}, R_{3″}, and R_{4″} are each —H, B₁, B₂, and B₃ are each —CH₂CH₂CH₂—, and X₁, X₂, and X₃ are each a sulfo group carboxyl group (—SO₃H).

### Example 4-2: Preparation of a Compound Having an Unshared Electron Pair for a Perovskite Solar Cell

(1) 223.65 mmol of a compound represented by [Chemical Formula 3-2] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-2] below.
(4) The prepared compound represented by [Chemical Formula 2-2] below was dissolved in 50 ml of anhydrous dimethyl formamide, and 1,3-dibromopropane, potassium carbonate, and potassium iodide were added. The mixture was stirred at 110° C. for 10 hours to prepare a second reaction product. At this time, 1,3-dibromopropane was added such that the compound represented by [Chemical Formula 2-2] below and 1,3-dibromopropane were in a molar ratio of 1:3.3; potassium carbonate was added such that the compound represented by [Chemical Formula 2-2] below and potassium carbonate were in a molar ratio of 1:6; and potassium iodide was added such that the compound represented by [Chemical Formula 2-2] below and potassium iodide were in a molar ratio of 1:0.2.
(5) The prepared second reaction product was extracted with dichloromethane, and the organic phase of the extract was concentrated under reduced pressure to prepare a concentrate. Then, the concentrate was purified through silica gel chromatography and dried to obtain a dried product.
(6) The obtained dried product was dissolved in a mixed solvent containing ethanol and water, and sodium sulfite was added. The mixture was stirred and reacted at 80° C. for 6 hours to prepare a third reaction product. At this time, sodium sulfite was added such that the compound represented by [Chemical Formula 2-2] below and sodium sulfite were in a molar ratio of 1:3.3.
(7) The third reaction product thus obtained was cooled to 25° C., and a 6 N hydrochloric acid (HCl) solution (containing 100 mmol of HCl) was added and stirred to prepare a fourth reaction product. The obtained fourth reaction product was then filtered to manufacture a compound having an unshared electron pair for a perovskite solar cell represented by [Chemical Formula 1-22] below. At this time, hydrochloric acid (HCl) was added such that the compound represented by [Chemical Formula 2-2] below and hydrochloric acid were in a molar ratio of 1:7.

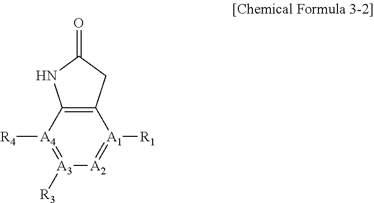

In [Chemical Formula 3-2], A₁, A₃, and A₄ are each carbon (C), A₂ is nitrogen (N), and R₁, R₃, and R₄ are each —H.

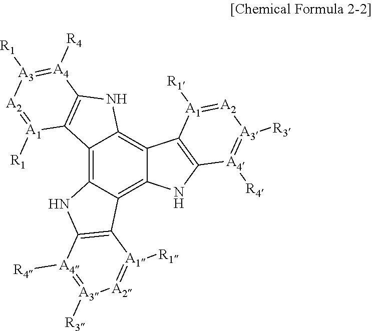

In [Chemical Formula 2-2], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), and R₁, R₃, R₄, R_{1′}, R_{3′}, R_{4′}, R_{1″}, R_{3″}, and R_{4″} are each —H.

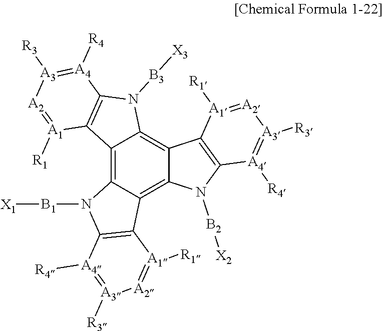

In [Chemical Formula 1-22], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), R₁, R₃, R₄, R_{1′}, R_{3′}, R_{4′}, R_{1″}, R_{3″}, and R_{4″} are each —H, B₁, B₂, and B₃ are each —CH₂CH₂CH₂—, and X₁, X₂, and X₃ are each a sulfo group (—SO₃H).

### Example 4-3: Preparation of a Compound Having an Unshared Electron Pair for a Perovskite Solar Cell

(1) 223.65 mmol of a compound represented by [Chemical Formula 3-3] below was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare a compound represented by [Chemical Formula 2-3] below.
(4) The prepared compound represented by [Chemical Formula 2-3] below was dissolved in 50 ml of anhydrous dimethyl formamide, and 1,3-dibromopropane, potassium carbonate, and potassium iodide were added. The mixture was stirred at 110° C. for 10 hours to prepare a second reaction product. At this time, 1,3-dibromopropane was added such that the compound represented by [Chemical Formula 2-3] below and 1,3-dibromopropane were in a molar ratio of 1:3.3; potassium carbonate was added such that the compound represented by [Chemical Formula 2-3] below and potassium carbonate were in a molar ratio of 1:6; and potassium iodide was added such that the compound represented by [Chemical Formula 2-3] below and potassium iodide were in a molar ratio of 1:0.2.
(5) The prepared second reaction product was extracted with dichloromethane, and the organic phase of the extract was concentrated under reduced pressure to prepare a concentrate. Then, the concentrate was purified through silica gel chromatography and dried to obtain a dried product.
(6) The obtained dried product was dissolved in a mixed solvent containing ethanol and water, and sodium sulfite was added. The mixture was stirred and reacted at 80° C. for 6 hours to prepare a third reaction product. At this time, sodium sulfite was added such that the compound represented by [Chemical Formula 2-3] below and sodium sulfite were in a molar ratio of 1:3.3.
(7) The third reaction product thus obtained was cooled to 25° C., and a 6 N hydrochloric acid (HCl) solution (containing 100 mmol of HCl) was added and stirred to prepare a fourth reaction product. The obtained fourth reaction product was then filtered to manufacture a compound having an unshared electron pair for a perovskite solar cell represented by [Chemical Formula 1-23] below. At this time, hydrochloric acid (HCl) was added so that the compound represented by [Chemical Formula 2-3] below and hydrochloric acid were in a molar ratio of 1:7.

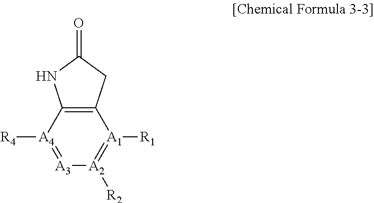

In [Chemical Formula 3-3], A₁, A₂, and A₄ are each carbon (C), A₃ is nitrogen (N), and R₁, R₂, and R₄ are each —H.

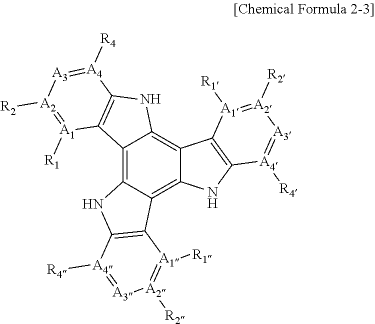

In [Chemical Formula 2-3], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), and R₁, R₂, R₄, R_{1′}, R_{2′}, R_{4′}, R_{1″}, R_{2″}, and R_{4″} are each —H.

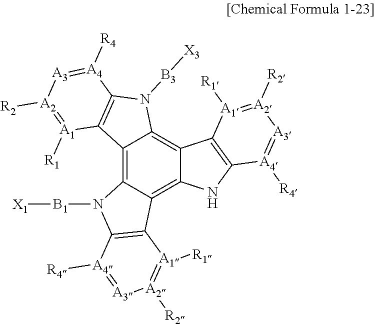

In [Chemical Formula 1-23], A₁, A₂, A₄, A_{1′}, A_{2′}, A_{4′}, A_{1″}, A_{2″}, and A_{4″} are each carbon (C), A₃, A_{3′}, and A_{3″} are each nitrogen (N), R₁, R₂, R₄, R_{1′}, R_{2′}, R_{4′}, R_{1″}, R_{2″}, and R_{4″} are each —H, B₁, B₂, and B₃ are each —CH₂CH₂CH₂—, and X₁, X₂, and X₃ are each a sulfo group (—SO₃H).

### Example 4-4: Preparation of a Compound Having an Unshared Electron Pair for a Perovskite Solar Cell

(1) 223.65 mmol of the compound represented by [Chemical Formula 3-2] was added to 50 ml of phosphoryl chloride, and the mixture was refluxed with stirring at 150° C. for 24 hours to prepare a first reaction product.
(2) The prepared first reaction product was neutralized with sodium hydroxide, the resulting precipitate was filtered to obtain a filtrate, and the obtained filtrate was purified through silica gel column chromatography (eluent: dichloromethane) to prepare a purified product.
(3) The prepared purified product was concentrated under reduced pressure and recrystallized using acetone to prepare the compound represented by [Chemical Formula 2-2].
(4) The prepared compound represented by [Chemical Formula 2-2] was dissolved in 50 ml of anhydrous dimethyl formamide, and 1,3-dibromobutane, potassium carbonate, and potassium iodide were added. The mixture was stirred at 110° C. for 10 hours to prepare a second reaction product. At this time, 1,3-dibromobutane was added such that the compound represented by [Chemical Formula 2-2] and 1,3-dibromobutane were in a molar ratio of 1:3.3; potassium carbonate was added such that the compound represented by [Chemical Formula 2-2] and potassium carbonate were in a molar ratio of 1:6; and potassium iodide was added such that the compound represented by [Chemical Formula 2-2] and potassium iodide were in a molar ratio of 1:0.2.
(5) The prepared second reaction product was extracted with dichloromethane, and the organic phase of the extract was concentrated under reduced pressure to prepare a concentrate. Then, the concentrate was purified through silica gel chromatography and dried to obtain a dried product.
(6) The obtained dried product was dissolved in a mixed solvent containing ethanol and water, and sodium sulfite was added. The mixture was stirred and reacted at 80° C. for 6 hours to prepare a third reaction product. At this time, sodium sulfite was added such that the compound represented by [Chemical Formula 2-2] and sodium sulfite were in a molar ratio of 1:3.3.
(7) The third reaction product thus obtained was cooled to 25° C., and a 6 N hydrochloric acid (HCl) solution (containing 100 mmol of HCl) was added and stirred to prepare a fourth reaction product. The obtained fourth reaction product was then filtered to manufacture a compound having an unshared electron pair for a perovskite solar cell represented by [Chemical Formula 1-24] below. At this time, hydrochloric acid (HCl) was added such that the compound represented by [Chemical Formula 2-2] and hydrochloric acid were in a molar ratio of 1:7.

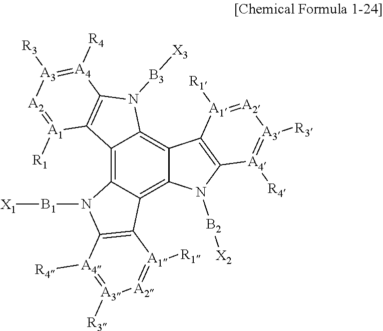

In [Chemical Formula 1-24], A₁, A₃, A₄, A_{1′}, A_{3′}, A_{4′}, A_{1″}, A_{3″}, and A_{4″} are each carbon (C), A₂, A_{2′}, and A_{2″} are each nitrogen (N), R₁, R₃, R₄, R_{1′}, R_{3′}, R_{4′}, R_{1″}, R_{3″}, and R_{4″} are each —H, B₁, B₂, and B₃ are each —CH₂CH₂CH₂CH₂—, and X₁, X₂, and X₃ are each a sulfo group (—SO₃H).

### Comparative Example 1: Preparation of a Compound Represented by Chemical Formula 4

A compound represented by [Chemical Formula 4] below was prepared.

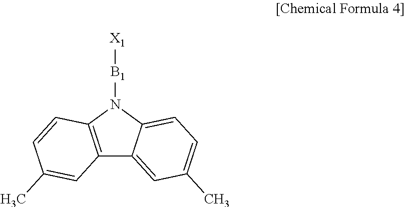

In [Chemical Formula 4], B₁ is-CH₂CH₂CH₂CH₂—, and X₁ is a dihydroxyphosphoryl group (—P═O(OH)₂).

### Comparative Example 2: Preparation of a Compound Represented by Chemical Formula 5

A compound represented by [Chemical Formula 5] below was prepared.

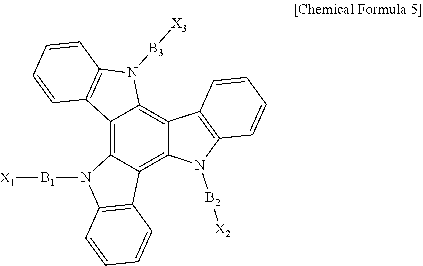

In [Chemical Formula 5], B₁, B₂, and B₃ are each —CH₂CH₂CH₂—, and X₁, X₂, and X₃ are each a dihydroxyphosphoryl group (—P═O(OH)₂).

### Experimental Example 1: Measurement of Water Contact Angle

An indium tin oxide (ITO) conductive transparent substrate was prepared, and on one surface of the prepared substrate, a compound having an unshared electron pair for a perovskite solar cell manufactured in Examples 2-1 and 2-3, a compound represented by [Chemical Formula 4] prepared in Comparative Example 1, and a compound represented by [Chemical Formula 5] prepared in Comparative Example 2 were each spin-coated to form an organic interfacial layer. A 3 μL drop of distilled water was then placed on the formed organic interfacial layer, and the water contact angle was measured. The results are shown in Table 1 below.

TABLE 1

| | Water Contact |
|---|---|
| Classification | Angle |
| Example 2-1 | 60.1° |
| Example 2-3 | 61.2° |
| Comparative | 85.1° |
| Example 1 | |
| Comparative | 79.4° |
| Example 2 | |

As can be seen from Table 1, the compounds having an unshared electron pair for a perovskite solar cell manufactured in Examples 2-1 and 2-3 exhibited lower water contact angles than the compound represented by [Chemical Formula 4] prepared in Comparative Example 1 and the compound represented by [Chemical Formula 5] prepared in Comparative Example 2. This confirms that hydrophilicity was enhanced, thereby improving coatability.

### Preparation Example 1-1: Manufacture of a Perovskite Solar Cell

(1) An indium tin oxide (ITO) conductive transparent substrate (thickness: 180 μm) was prepared, and a 20 nm-thick recombination layer (ITO) was formed on the prepared transparent substrate by a sputtering process.
(2) On the recombination layer, nickel oxide (NiOx) having a thickness of 20 nm was deposited by a sputter vacuum deposition method to form a hole transport layer.
(3) The compound having an unshared electron pair for a perovskite solar cell prepared in Example 1-1 was mixed with ethanol to prepare an organic interfacial layer-forming solution containing the compound having an unshared electron pair for a perovskite solar cell prepared in Example 1-1 at a concentration of 1 mM.
(4) The prepared organic interfacial layer-forming solution was spin-coated on the hole transport layer at a speed of 3000 rpm for 30 seconds, and then heat-treated at a temperature of 100° C. for 20 minutes to form an organic interfacial layer having a thickness of 2 nm.
(5) A yellow light-absorbing layer solution prepared by dissolving precursors in dimethylformamide (DMF) and dimethyl sulfoxide (DMSO) was spin-coated on the organic interfacial layer, and then heat-treated at a temperature of 150° C. for 10 minutes to form a perovskite light-absorbing layer (CSFAPbIₓBr₃₋ₓ (0≤x≤3)) having a perovskite crystal structure and a thickness of 400 nm.
(6) An electron transport layer (SnO₂) having an average thickness of 10 nm was formed on the perovskite light-absorbing layer by an atomic layer deposition (ALD) process.
(7) A transparent electrode (IZO) having a thickness of 75 nm was formed on the electron transport layer by a sputtering process.
(8) A metal electrode was formed on the transparent electrode by depositing silver (Ag) to a thickness of 100 nm at a pressure of 1×10⁻⁷ torr through a screen printing process, thereby manufacturing a perovskite solar cell in which the transparent substrate, recombination layer, hole transport layer, organic interfacial layer, perovskite light-absorbing layer, electron transport layer, transparent electrode, and metal electrode were sequentially stacked.

### Preparation Example 1-2: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 1-2 was used, and the perovskite solar cell was finally manufactured.

### Preparation Example 1-3: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 1-3 was used, and the perovskite solar cell was finally manufactured.

### Preparation Example 1-4: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 1-4 was used, and the perovskite solar cell was finally manufactured.

### Preparation Example 1-5: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 1-5 was used, and the perovskite solar cell was finally manufactured.

### Preparation Example 1-6: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 1-6 was used, and the perovskite solar cell was finally manufactured.

### Preparation Example 1-7: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 1-7 was used, and the perovskite solar cell was finally manufactured.

### Preparation Example 1-8: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 1-8 was used, and the perovskite solar cell was finally manufactured.

### Preparation Example 1-9: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 1-9 was used, and the perovskite solar cell was finally manufactured.

### Preparation Example 2-1: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 2-1 was used, and the perovskite solar cell was finally manufactured.

### Preparation Example 2-2: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 2-3 was used, and the perovskite solar cell was finally manufactured.

### Preparation Example 2-3: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 2-5 was used, and the perovskite solar cell was finally manufactured.

### Preparation Example 2-4: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 2-6 was used, and the perovskite solar cell was finally manufactured.

### Preparation Example 2-5: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 2-7 was used, and the perovskite solar cell was finally manufactured.

### Preparation Example 2-6: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 2-8 was used, and the perovskite solar cell was finally manufactured.

### Preparation Example 3-1: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 3-1 was used, and the perovskite solar cell was finally manufactured.

### Preparation Example 3-2: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 3-2 was used, and the perovskite solar cell was finally manufactured.

### Preparation Example 3-3: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 3-3 was used, and the perovskite solar cell was finally manufactured.

### Preparation Example 4-1: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 4-1 was used, and the perovskite solar cell was finally manufactured.

### Preparation Example 4-2: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 4-2 was used, and the perovskite solar cell was finally manufactured.

### Preparation Example 4-3: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 4-3 was used, and the perovskite solar cell was finally manufactured.

### Preparation Example 4-4: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound having an unshared electron pair for a perovskite solar cell prepared in Example 4-4 was used, and the perovskite solar cell was finally manufactured.

### Comparative Preparation Example 1: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound represented by [Chemical Formula 4] prepared in Comparative Example 1 was used, and the perovskite solar cell was finally manufactured.

### Comparative Preparation Example 2: Manufacture of a Perovskite Solar Cell

A perovskite solar cell was manufactured in the same manner as in Preparation Example 1-1. However, unlike in Preparation Example 1-1, instead of using the compound having an unshared electron pair for a perovskite solar cell prepared in Example 1-1, the compound represented by [Chemical Formula 5] prepared in Comparative Example 2 was used, and the perovskite solar cell was finally manufactured.

### Experimental Example 2: Measurement of Performance of Solar Cells

For each of the perovskite solar cells manufactured in Preparation Examples 1-1 to 1-9 and Comparative Preparation Examples 1 to 2, efficiency was measured using a solar simulator and JV Keithley equipment, based on an initial JV curve, and the results are shown in Table 2 below.

TABLE 2

| | | Short-circuit | | | |
|---|---|---|---|---|---|
| | Open- | current | | Power | |
| | circuit | density | Fill | conversion | Series |
| | voltage | (Jsc, | factor | efficiency | resistance |
| Classification | (Voc, V) | mA/cm²) | (FF) | (%) | (Rₛ, Ω) |
| Preparation | 1.137 | 19.66 | 76.32 | 17.08 | 45.82 |
| Example 1-1 | | | | | |
| Preparation | 1.137 | 19.43 | 76.85 | 16.94 | 45.75 |
| Example 1-2 | | | | | |
| Preparation | 1.123 | 19.47 | 78.29 | 17.21 | 43.57 |
| Example 1-3 | | | | | |
| Preparation | 1.128 | 19.74 | 77.69 | 17.01 | 45.02 |
| Example 1-4 | | | | | |
| Preparation | 1.112 | 19.34 | 76.53 | 16.46 | 44.13 |
| Example 1-5 | | | | | |
| Preparation | 1.112 | 19.03 | 75.94 | 16.14 | 44.79 |
| Example 1-6 | | | | | |
| Preparation | 1.107 | 19.21 | 77.71 | 16.54 | 37.7 |
| Example 1-7 | | | | | |
| Preparation | 1.112 | 19.41 | 76.90 | 16.60 | 43.7 |
| Example 1-8 | | | | | |
| Preparation | 1.090 | 19.38 | 78.6 | 16.61 | 39.1 |
| Example 1-9 | | | | | |
| Comparative | 1.074 | 16.07 | 72.05 | 12.62 | 52.98 |
| Preparation | | | | | |
| Example 1 | | | | | |
| Comparative | 1.088 | 16.91 | 75.11 | 13.71 | 47.06 |
| Preparation | | | | | |
| Example 2 | | | | | |

As can be seen from Table 2, the perovskite solar cells manufactured in Preparation Examples 1-1 to 1-9 exhibited superior power conversion efficiency (PCE) compared to the perovskite solar cells manufactured in Comparative Preparation Examples 1 to 2.

In addition, among the perovskite solar cells manufactured in Preparation Examples 1-1 to 1-5, the perovskite solar cell manufactured in Preparation Example 1-3 exhibited an even higher power conversion efficiency (PCE).

In addition, among the perovskite solar cells manufactured in Preparation Examples 1-6 to 1-9, the perovskite solar cell manufactured in Preparation Example 1-9 exhibited an even higher power conversion efficiency (PCE).

### Experimental Example 3: Measurement of Performance of Solar Cells

For each of the perovskite solar cells manufactured in Preparation Examples 2-1 to 2-6 and Comparative Preparation Examples 1 to 2, efficiency was measured using a solar simulator and JV Keithley equipment, based on an initial JV curve, and the results are shown in Table 3 below.

TABLE 3

| | | Short-circuit | | | |
|---|---|---|---|---|---|
| | Open- | current | | Power | |
| | circuit | density | Fill | conversion | Series |
| | voltage | (Jsc, | factor | efficiency | resistance |
| Classification | (Voc, V) | mA/cm²) | (FF) | (%) | (Rₛ, Ω) |
| Preparation | 1.137 | 19.66 | 76.32 | 17.08 | 45.82 |
| Example 2-1 | | | | | |
| Preparation | 1.137 | 19.43 | 76.85 | 16.94 | 45.75 |
| Example 2-2 | | | | | |
| Preparation | 1.112 | 19.03 | 75.94 | 16.14 | 44.79 |
| Example 2-3 | | | | | |
| Preparation | 1.112 | 19.34 | 76.53 | 16.46 | 44.13 |
| Example 2-4 | | | | | |
| Preparation | 1.123 | 19.47 | 78.29 | 17.21 | 43.57 |
| Example 2-5 | | | | | |
| Preparation | 1.090 | 19.38 | 78.6 | 16.61 | 39.10 |
| Example 2-6 | | | | | |
| Comparative | 1.074 | 16.07 | 72.05 | 12.62 | 52.98 |
| Preparation | | | | | |
| Example 1 | | | | | |
| Comparative | 1.088 | 16.91 | 75.11 | 13.71 | 47.06 |
| Preparation | | | | | |
| Example 2 | | | | | |

As can be seen from Table 3, the perovskite solar cells manufactured in Preparation Examples 2-1 to 2-6 exhibited superior power conversion efficiency (PCE) compared to the perovskite solar cells manufactured in Comparative Preparation Examples 1 to 2.

In addition, among the perovskite solar cells manufactured in Preparation Examples 2-1 to 2-2, the perovskite solar cell manufactured in Preparation Example 2-1 exhibited an even higher power conversion efficiency (PCE).

In addition, among the perovskite solar cells manufactured in Preparation Examples 2-3 to 2-6, the perovskite solar cell manufactured in Preparation Example 2-5 exhibited an even higher power conversion efficiency (PCE).

### Experimental Example 4: Measurement of Performance of Solar Cells

For each of the perovskite solar cells manufactured in Preparation Examples 3-1 to 3-3 and Comparative Preparation Examples 1 to 2, efficiency was measured using a solar simulator and JV Keithley equipment, based on an initial JV curve, and the results are shown in Table 4 below.

TABLE 4

| | | Short-circuit | | | |
|---|---|---|---|---|---|
| | Open- | current | | Power | |
| | circuit | density | Fill | conversion | Series |
| | voltage | (Jsc, | factor | efficiency | resistance |
| Classification | (Voc, V) | mA/cm²) | (FF) | (%) | (Rₛ, Ω) |
| Preparation | 1.137 | 19.75 | 78.80 | 17.72 | 41.00 |
| Example 3-1 | | | | | |
| Preparation | 1.138 | 19.72 | 78.12 | 17.51 | 40.62 |
| Example 3-2 | | | | | |
| Preparation | 1.128 | 19.77 | 79.61 | 17.77 | 37.91 |
| Example 3-3 | | | | | |
| Comparative | 1.074 | 16.07 | 72.05 | 12.62 | 52.98 |
| Preparation | | | | | |
| Example 1 | | | | | |
| Comparative | 1.088 | 16.91 | 75.11 | 13.71 | 47.06 |
| Preparation | | | | | |
| Example 2 | | | | | |

As can be seen from Table 4, the perovskite solar cells manufactured in Preparation Examples 3-1 to 3-3 exhibited superior power conversion efficiency (PCE) compared to the perovskite solar cells manufactured in Comparative Preparation Examples 1 to 2.

In addition, among the perovskite solar cells manufactured in Preparation Examples 3-1 to 3-3, the perovskite solar cell manufactured in Preparation Example 3-3 exhibited an even higher power conversion efficiency (PCE).

### Experimental Example 5: Measurement of Performance of Solar Cells

For each of the perovskite solar cells manufactured in Preparation Examples 4-1 to 4-4 and Comparative Preparation Examples 1 to 2, efficiency was measured using a solar simulator and JV Keithley equipment, based on an initial JV curve, and the results are shown in Table 5 below.

TABLE 5

| | | Short- | | | |
|---|---|---|---|---|---|
| | | circuit | | | |
| | Open- | current | | Power | |
| | circuit | density | Fill | conversion | Series |
| | voltage | (Jsc, | factor | efficiency | resistance |
| Classification | (Voc, V) | mA/cm²) | (FF) | (%) | (Rₛ, Ω) |
| Preparation | 1.101 | 19.69 | 82.23 | 17.81 | 33.74 |
| Example 4-1 | | | | | |
| Preparation | 1.134 | 19.63 | 81.38 | 18.12 | 33.6 |
| Example 4-2 | | | | | |
| Preparation | 1.108 | 19.72 | 77.45 | 16.92 | 38.7 |
| Example 4-3 | | | | | |
| Preparation | 1.128 | 19.77 | 79.61 | 17.77 | 37.91 |
| Example 4-4 | | | | | |
| Comparative | 1.074 | 16.07 | 72.05 | 12.62 | 52.98 |
| Preparation | | | | | |
| Example 1 | | | | | |
| Comparative | 1.088 | 16.91 | 75.11 | 13.71 | 47.06 |
| Preparation | | | | | |
| Example 2 | | | | | |

As can be seen from Table 5, the perovskite solar cells manufactured in Preparation Examples 4-1 to 4-4 exhibited superior power conversion efficiency (PCE) compared to the perovskite solar cells manufactured in Comparative Preparation Examples 1 to 2.

In addition, among the perovskite solar cells manufactured in Preparation Examples 4-1 to 4-4, the perovskite solar cell manufactured in Preparation Example 4-2 exhibited an even higher power conversion efficiency (PCE).

The present disclosure has been illustrated and described with reference to specific examples. However, the present disclosure is not limited to the above-described embodiments, and various modifications and changes can be made by those skilled in the art without departing from the spirit of the invention as set forth in the following claims.

What is claimed is:

1. A compound having an unshared electron pair for a perovskite solar cell, represented by [Chemical Formula 1]: 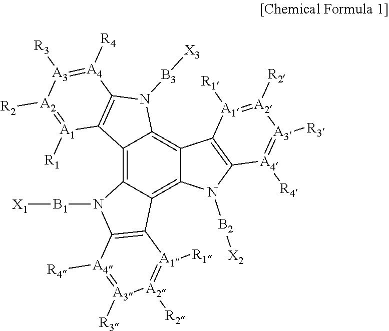
wherein, in [Chemical Formula 1], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N);
R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group;
B₁, B₂, and B₃ are each independently a single bond, a C6-C18 arylene group, or a C5-C18 heteroarylene group; and
X₁, X₂, and X₃ are each independently a boronic acid group (—B(OH)₂), a boronic ester group, a dihydroxyphosphoryl group (—P═O(OH)₂), a trihalosilyl group (—SiX₃), a trialkoxysilyl group (—Si(OR)₃), a carboxyl group (—COOH), or a sulfo group (—SO₃H).

2. The compound having an unshared electron pair for a perovskite solar cell according to claim 1,
wherein the compound having an unshared electron pair for a perovskite solar cell represented by [Chemical Formula 1] is a compound having an unshared electron pair for a perovskite solar cell represented by [Chemical Formula 1-25] below: 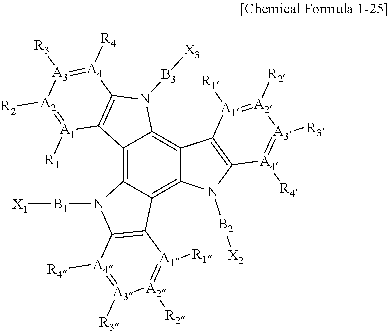
wherein, in [Chemical Formula 1-25], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N);
R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group;
B₁, B₂, and B₃ are each independently —CH₂—, —CH₂CH₂—, —CH₂CH₂CH₂—, —CH₂CH₂CH₂CH₂—, —CH₂CH₂CH₂CH₂CH₂—, 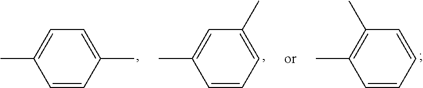
X₁, X₂, and X₃ are each independently a boronic acid group (—B(OH)₂), 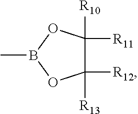
a dihydroxyphosphoryl group (—P═O(OH)₂), a carboxyl group (—COOH), or a sulfo group (—SO₃H); and
R₁₀, R₁₁, R₁₂, and R¹³ are each independently —H, a C1-C20 linear alkyl group, or a C3-C20 branched alkyl group.

3. The compound having an unshared electron pair for a perovskite solar cell according to claim 1,
wherein the compound having an unshared electron pair for a perovskite solar cell represented by [Chemical Formula 1] is a compound having an unshared electron pair for a perovskite solar cell represented by [Chemical Formula 1-1], [Chemical Formula 1-2], [Chemical Formula 1-3], or [Chemical Formula 1-4] below: 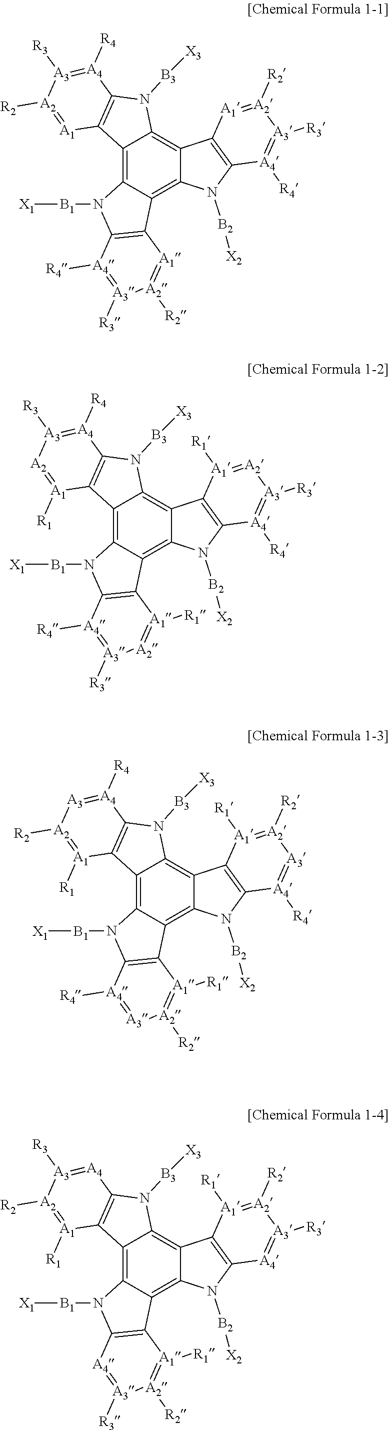
wherein, in [Chemical Formula 1-1], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently 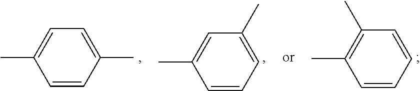
and X₁, X₂, and X₃ are each a boronic acid group (—B(OH)₂),
wherein, in [Chemical Formula 1-2], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently 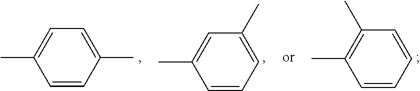
and X₁, X₂, and X₃ are each a boronic acid group (—B(OH)₂),
wherein, in [Chemical Formula 1-3], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently 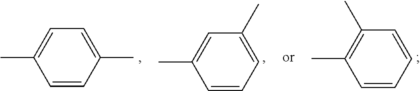
and X₁, X₂, and X₃ are each a boronic acid group (—B(OH)₂), and
wherein, in [Chemical Formula 1-4], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently 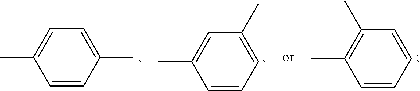
and X₁, X₂, and X₃ are each a boronic acid group (—B(OH)₂).

4. The compound having an unshared electron pair for a perovskite solar cell according to claim 1,
wherein the compound having an unshared electron pair for a perovskite solar cell represented by [Chemical Formula 1] is a compound having an unshared electron pair for a perovskite solar cell represented by [Chemical Formula 1-6], [Chemical Formula 1-7], [Chemical Formula 1-8], or [Chemical Formula 1-9] below: 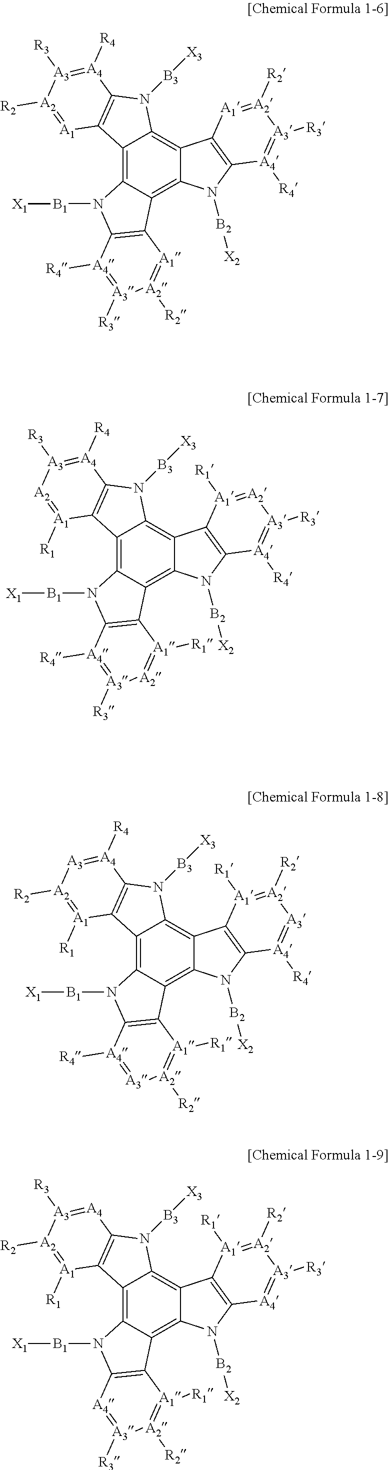
wherein, in [Chemical Formula 1-6], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently 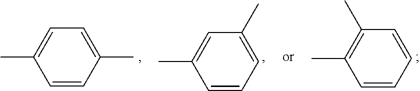
X₁, X₂, and X₃ are each 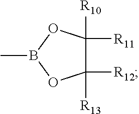
and R₁₀, R₁₁, R₁₂, and R¹³ are each independently —H, a C1-C20 linear alkyl group, or a C3-C20 branched alkyl group,
wherein, in [Chemical Formula 1-7], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently 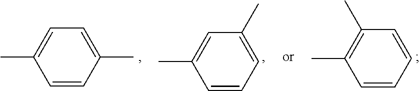
X₁, X₂, and X₃ are each 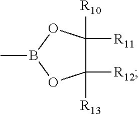
and R₁₀, R₁₁, R₁₂, and R¹³ are each independently —H, a C1-C20 linear alkyl group, or a C3-C20 branched alkyl group,
wherein, in [Chemical Formula 1-8], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently 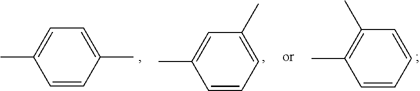
X₁, X₂, and X₃ are each 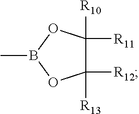
and R₁₀, R₁₁, R₁₂, and R¹³ are each independently —H, a C1-C20 linear alkyl group, or a C3-C20 branched alkyl group, and
wherein, in [Chemical Formula 1-9], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently 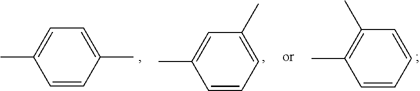
X₁, X₂, and X₃ are each 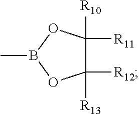
and R₁₀, R₁₁, R₁₂, and R¹³ are each independently —H, a C1-C20 linear alkyl group, or a C3-C20 branched alkyl group.

5. The compound having an unshared electron pair for a perovskite solar cell according to claim 1,
wherein the compound having an unshared electron pair for a perovskite solar cell represented by [Chemical Formula 1] is a compound having an unshared electron pair for a perovskite solar cell represented by [Chemical Formula 1-10], [Chemical Formula 1-11], [Chemical Formula 1-12], or [Chemical Formula 1-13] below: 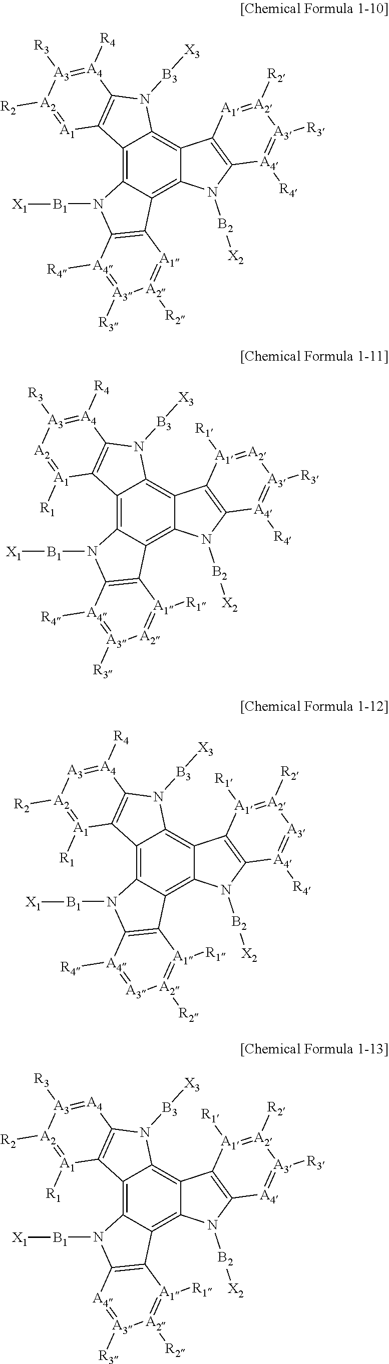
wherein, in [Chemical Formula 1-10], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently —CH₂CH₂—, —CH₂CH₂CH₂—, or —CH₂CH₂CH₂CH₂—; and X₁, X₂, and X₃ are each dihydroxyphosphoryl group (—P═O(OH)₂),
wherein, in [Chemical Formula 1-11], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently —CH₂CH₂—, —CH₂CH₂CH₂—, or —CH₂CH₂CH₂CH₂—; and X₁, X₂, and X₃ are each dihydroxyphosphoryl group (—P═O(OH)₂),
wherein, in [Chemical Formula 1-12], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently —CH₂CH₂—, —CH₂CH₂CH₂—, or —CH₂CH₂CH₂CH₂—; and X₁, X₂, and X₃ are each dihydroxyphosphoryl group (—P═O(OH)₂), and
wherein, in [Chemical Formula 1-13], A₁, A₂, A₃, A₁, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently —CH₂CH₂—, —CH₂CH₂CH₂—, or —CH₂CH₂CH₂CH₂—; and X₁, X₂, and X₃ are each dihydroxyphosphoryl group (—P═O(OH)₂).

6. The compound having an unshared electron pair for a perovskite solar cell according to claim 1,
wherein the compound having an unshared electron pair for a perovskite solar cell represented by [Chemical Formula 1] is a compound having an unshared electron pair for a perovskite solar cell represented by [Chemical Formula 1-14], [Chemical Formula 1-15], [Chemical Formula 1-16], or [Chemical Formula 1-17] below: 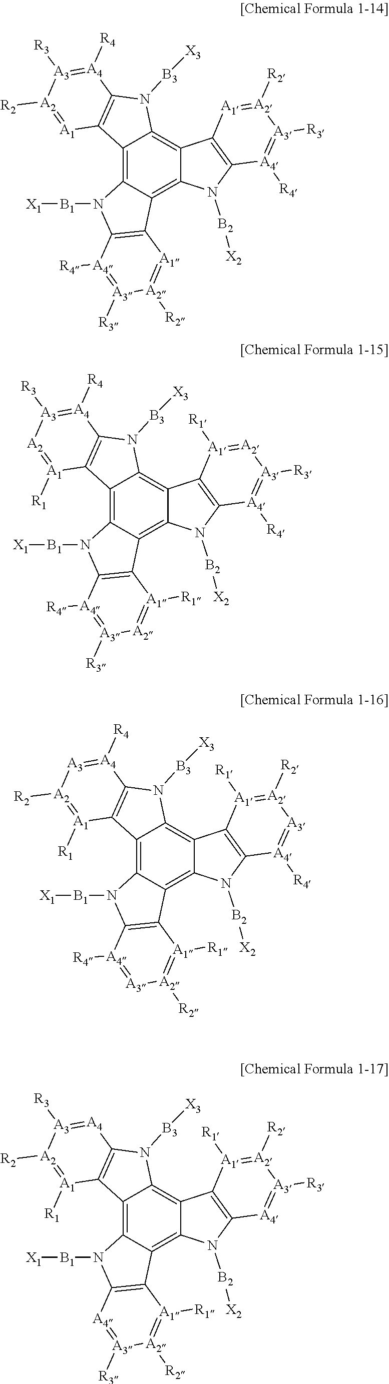
wherein, in [Chemical Formula 1-14], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently 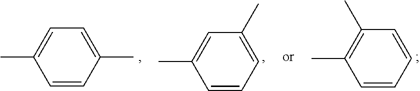
and X₁, X₂, and X₃ are each a dihydroxyphosphoryl group (—P═O(OH)₂),
wherein, in [Chemical Formula 1-15], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently 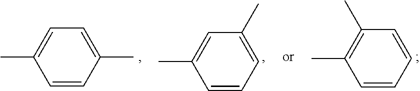
and X₁, X₂, and X₃ are each a dihydroxyphosphoryl group (—P═O(OH)₂),
wherein, in [Chemical Formula 1-16], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently 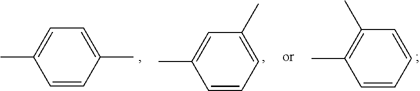
and X₁, X₂, and X₃ are each a dihydroxyphosphoryl group (—P═O(OH)₂), and
wherein, in [Chemical Formula 1-17], A₁, A₂, A₃, A_{1′}, A_{2′}, A_{3′}, A_{1″}, A_{2″}, independently and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently 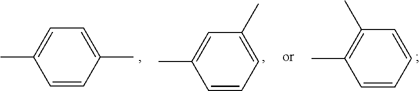
and X₁, X₂, and X₃ are each a dihydroxyphosphoryl group (—P═O(OH)₂).

7. The compound having an unshared electron pair for a perovskite solar cell according to claim 1,
wherein the compound having an unshared electron pair for a perovskite solar cell represented by [Chemical Formula 1] is a compound having an unshared electron pair for a perovskite solar cell represented by [Chemical Formula 1-18], [Chemical Formula 1-19], [Chemical Formula 1-20], or [Chemical Formula 1-26] below: 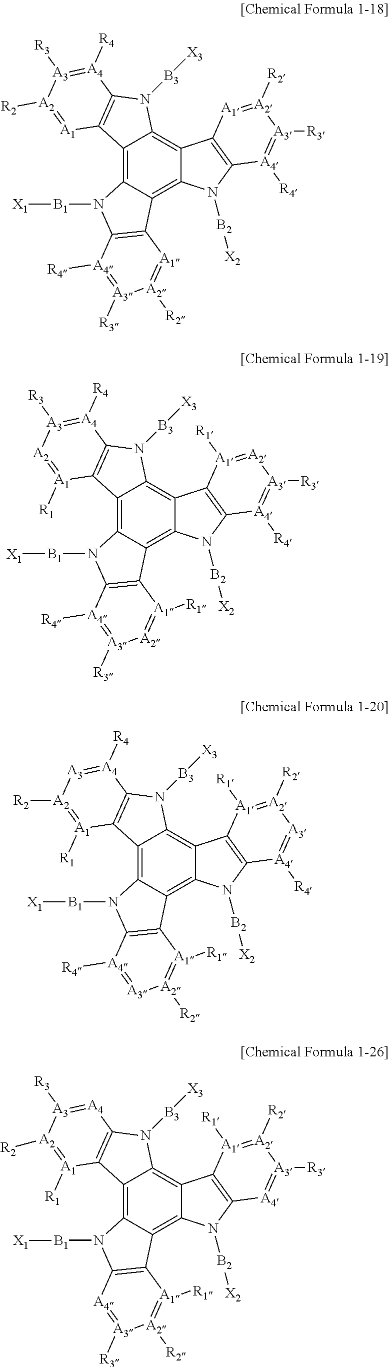
wherein, in [Chemical Formula 1-18], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each carboxyl group (—COOH),
wherein, in [Chemical Formula 1-19], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each carboxyl group (—COOH),
wherein, in [Chemical Formula 1-20], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each carboxyl group (—COOH), and
wherein, in [Chemical Formula 1-26], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each carboxyl group (—COOH).

8. The compound having an unshared electron pair for a perovskite solar cell according to claim 1,
wherein the compound having an unshared electron pair for a perovskite solar cell represented by [Chemical Formula 1] is a compound having an unshared electron pair for a perovskite solar cell represented by [Chemical Formula 1-21], [Chemical Formula 1-22], [Chemical Formula 1-23], or [Chemical Formula 1-27] below: 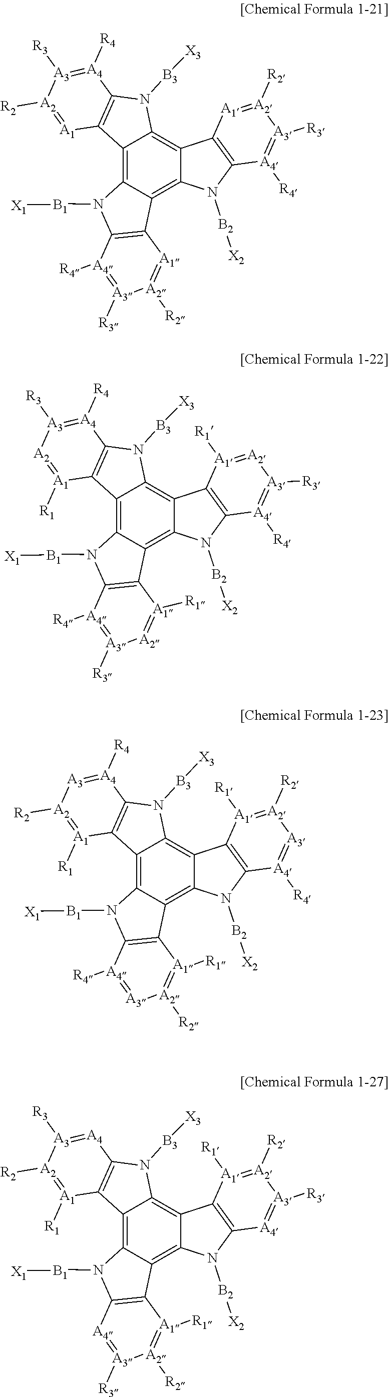
wherein, in [Chemical Formula 1-21], A₂, A₃, A₄, A_{2′}, A_{3′}, A_{4′}, A_{2″}, A_{3″}, and A_{4″} are each carbon (C); A₁, A_{1′}, and A_{1″} are each nitrogen (N); R₂, R₃, R₄, R_{2′}, R_{3′}, R_{4′}, R_{2″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each sulfo group (—SO₃H),
wherein, in [Chemical Formula 1-22], A₁, A₃, A₄, A_{1′}, A_{3′}, A_{4′}, A_{1″}, A_{3″}, and A_{4″} are each carbon (C); A₂, A_{2′}, and A_{2″} are each nitrogen (N); R₁, R₃, R₄, R_{1′}, R_{3′}, R_{4′}, R_{1″}, R_{3″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each sulfo group (—SO₃H),
wherein, in [Chemical Formula 1-23], A₁, A₂, A₄, A_{1′}, A_{2′}, A_{4′}, A_{1″}, A_{2″}, and A_{4″} are each carbon (C); A₃, A_{3′}, and A_{3″} are each nitrogen (N); R₁, R₂, R₄, R_{1′}, R_{2′}, R_{4′}, R_{1″}, R_{2″}, and R_{4″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each sulfo group (—SO₃H), and
wherein, in [Chemical Formula 1-27], A₁, A₂, A₃, A_{1′}, A_{2′}, A_{3′}, A_{1″}, A_{2″}, and A_{3″} are each carbon (C); A₄, A_{4′}, and A_{4″} are each nitrogen (N); R₁, R₂, R₃, R_{1′}, R_{2′}, R_{3′}, R_{1″}, R_{2″}, and R_{3″} are each independently —H, a C1-C20 linear alkyl group, or a C3-C20 branched alkyl group; B₁, B₂, and B₃ are each independently —CH₂—, —CH₂CH₂—, —CH₂CH₂CH₂—, or —CH₂CH₂CH₂CH₂—; and X₁, X₂, and X₃ are each sulfo group (—SO₃H).

9. A perovskite solar cell, comprising:
a laminate in which a hole transport layer, an organic interfacial layer, and a perovskite light-absorbing layer are sequentially stacked,
wherein the organic interfacial layer comprises a compound having an unshared electron pair for a perovskite solar cell represented by [Chemical Formula 1]: 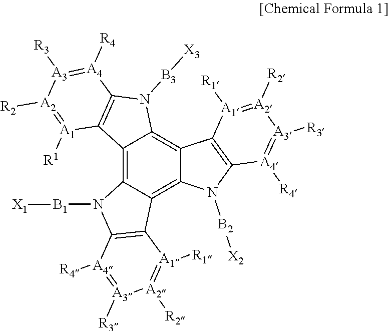
wherein, in [Chemical Formula 1], A₁, A₂, A₃, A₄, A_{1′}, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R_{1′}, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a boronic acid group (—B(OH)₂), a boronic ester group, a dihydroxyphosphoryl group (—P═O(OH)₂), a trihalosilyl group (—SiX₃), a trialkoxysilyl group (—Si(OR)₃), a carboxyl group (—COOH), or a sulfo group (—SO₃H).

10. The perovskite solar cell according to claim 9,
wherein the perovskite light-absorbing layer comprises a perovskite material represented by [Chemical Formula 1]: 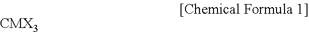
wherein, in [Chemical Formula 1], C is a monovalent cation, M is a divalent cation, and X is a monovalent anion.

11. A tandem perovskite solar cell, comprising:
a laminate in which a solar cell, a recombination layer, a hole transport layer, an organic interfacial layer, and a perovskite light-absorbing layer are sequentially stacked; and
wherein the organic interfacial layer comprises a compound having an unshared electron pair for a perovskite solar cell represented by [Chemical Formula 1]: 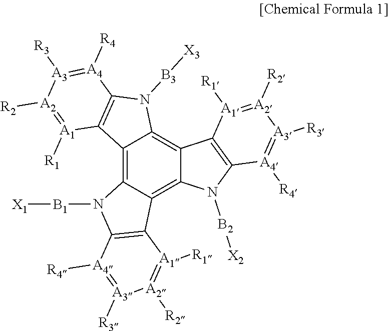
wherein, in [Chemical Formula 1], A₁, A₂, A₃, A₄, A₁, A_{2′}, A_{3′}, A_{4′}, A_{1″}, A_{2″}, A_{3″}, and A_{4″} are each independently carbon (C) or nitrogen (N), provided that at least one of A₁, A₂, A₃, and A₄ is nitrogen (N), at least one of A_{1′}, A_{2′}, A_{3′}, and A_{4′} is nitrogen (N), and at least one of A_{1″}, A_{2″}, A_{3″}, and A_{4″} is nitrogen (N); R₁, R₂, R₃, R₄, R₁, R_{2′}, R_{3′}, R_{4′}, R_{1″}, R_{2″}, R_{3″}, and R_{4″} are each independently —H, a C1-C20 linear alkyl group, or a C3-C20 branched alkyl group; B₁, B₂, and B₃ are each independently a single bond, a C6-C18 arylene group, or a C5-C18 heteroarylene group; and X₁, X₂, and X₃ are each independently a boronic acid group (—B(OH)₂), a boronic ester group, a dihydroxyphosphoryl group (—P═O(OH)₂), a trihalosilyl group (—SiX₃), a trialkoxysilyl group (—Si(OR)₃), a carboxyl group (—COOH), or a sulfo group (—SO₃H).

12. The tandem perovskite solar cell according to claim 11, wherein the solar cell is a polycrystalline silicon solar cell, a crystalline silicon solar cell, a perovskite solar cell, a gallium arsenide (GaAs) solar cell, a cadmium telluride (CdTe) solar cell, a CIGS (CuInGaSe) solar cell, a CZTS (Cu₂ZnSnS₄) solar cell, an organic solar cell, a dye-sensitized solar cell, or a group III-V compound solar cell.
